(19) United States
(12) Patent Application Publication
(10) Pub. No.: US 2025/0101483 A1
(43) Pub. Date: Mar. 27, 2025

(54) GLYCOENGINEERING OF THERMOTHELOMYCES HETEROTHALLICA

(71) Applicant: DYADIC INTERNATIONAL (USA), INC., Jupiter, FL (US)

(72) Inventors: Anne Huuskonen, Espoo (FI); Ronen Tchelet, Budapest (HU); Mark Aaron Emalfarb, Jupiter, FL (US); Noelia Valbuena Crespo, Seville (ES); Markku Saloheimo, Espoo (FI)

(21) Appl. No.: 18/833,874

(22) PCT Filed: Jan. 26, 2023
(86) PCT No.: PCT/IB2023/050667

§ 371 Date: Jul. 26, 2024

Related U.S. Application Data
(60) Provisional application No. 63/303,517, filed on Jan. 27, 2022.

(51) Int. Cl. C12P 21/00; C12N 1/14; C12N 9/10; C12N 9/24

(52) U.S. Cl. CPC C12P 21/005; C12N 1/14; C12N 9/1051; C12N 9/2402; C07K 2319/05; C12N 2510/00; C12Y 204/01132; C12Y 204/01255; C12Y 302/01113

(57) ABSTRACT

Thermothelomyces heterothallica (formerly Myceliophthora thermophila) genetically modified to produce glycoproteins with N-glycans of mammalian proteins (particularly human, companion animal and other animal proteins) are provided, comprising deletion or disruption of the alg3 gene, expression of ER-targeted Mannosidase 1 (alpha-1.2-Mannosidase), and expression of ER-targeted Glucosidase 2 alpha-subunit. The Th. heterothallica may also further comprise heterologous GlcNAc transferase 1 (GNT1), GlcNAc transferase 2 (GNT2), STT3 subunit of a heterologous oligosaccharyltransferase and galactosyltransferase.

### FIELD OF THE INVENTION

The present invention relates to genetically-modified Thermothelomyces heterothallica (formerly Myceliophthora thermophila) in which protein glycosylation pathways have been engineered with minimal disruption of endogenous genes to produce proteins with N-glycans similar to those of mammalian proteins, particularly human proteins.

### BACKGROUND OF THE INVENTION

Most therapeutic proteins require glycosylation to ensure proper folding, function, and activity. Glycosylation of therapeutic proteins is also particularly important for their immunogenicity. Therefore, such proteins cannot be produced in standard prokaryotic expression systems, which lack the necessary glycosylation machinery. Since glycosylation and other post-translational modifications are essential for therapeutic glycoproteins, most of them are currently produced in mammalian cells. However, fermentation processes based on mammalian cell culture (e.g., CHO, murine, or human cells) are typically very slow, require expensive nutrients and cofactors (e.g., bovine fetal serum or specific growth factors), often yield low product titers, and also are susceptible to infections which may contaminate the resulting protein product. Thus, there is a growing shift to serum-free expression systems. In particular, yeasts and fungi are being developed as alternative protein expression systems.

As eukaryotic organisms, yeast and fungi are able to perform post-translational modifications, including N- and O-glycosylation, but protein glycosylation in yeast and fungi is quite different from that in mammalian cells. To overcome these problems, the possibility of reengineering the N-glycosylation pathway has been explored, especially in the species most frequently used for the production of heterologous proteins (e.g., S. cerevisiae, Pichia pastoris, Yarrowia lipolytica, Hansenula polymorpha, and Aspergillus and Trichoderma species). However, protein yields still need improvements, and particularly there is a need to improve the glycosylation pattern, such that a high percentage of the produced proteins carry the desired glycoforms, namely, glycoforms of mammalian (particularly human and companion animals) proteins.

Parsaie Nasab et al., 2013, Appl Environ Microbiol., 79 (3): 997-1007 describe a synthetic N-glycosylation pathway to produce recombinant proteins carrying human N-glycans in Saccharomyces cerevisiae. A Δalg3 4alg11 double mutant strain was used, which was further genetically modified to express an artificial flippase, a protozoan oligosaccharyltransferase and Golgi-targeted human N-acetylglucosaminyltransferases I and II. The results confirmed the presence of the complex human N-glycan structure GlcNAc₂Man₃GlcNAc₂ on a secreted monoclonal antibody recombinantly expressed in the mutant strain. However, due to the interference of Golgi apparatus-localized mannosyltransferases, heterogeneity of N-linked glycans was observed.

The work by Parsaic Nasab et al. is also described in US 2011/0207214, disclosing cells modified to express lipid-linked oligosaccharide (LLO) flippase activity that is capable of flipping LLO comprising 1 mannose residue, 2 mannose residues and 3 mannose residues, from the cytosolic side to the lumenal side of an intracellular organelle, and further reviewed along with other related studies in De Wachter et al., 2018, Engineering of Yeast

Glycoprotein Expression. In: Advances in Biochemical Engineering/Biotechnology. Springer, Berlin, Heidelberg.

U.S. Pat. No. 7,029,872, 7,326,681, 7,629,163, 7,981,660 disclose cell lines having genetically modified glycosylation pathways that allow them to carry out a sequence of enzymatic reactions, which mimic the processing of glycoproteins in humans. Eukaryotes such as unicellular and multicellular fungi, which ordinarily produce high-mannose-containing N-glycans, are modified to produce N-glycans such as MansGlcNAcz or other structures along human glycosylation pathways.

U.S. Pat. No. 7,449,308, 7,935,513 disclose eukaryotic host cells having modified oligosaccharides which may be modified further by heterologous expression of a set of glycosyltransferases, sugar transporters and mannosidases to become host-strains for the production of mammalian, e.g., human therapeutic glycoproteins. N-glycans made in the engineered host cells have a Man₅GlcNAc₂ core structure which may then be modified further by heterologous expression of one or more enzymes, e.g., glycosyltransferases, sugar transporters and mannosidases, to yield human-like glycoproteins.

U.S. Pat. No. 7,795,002 discloses eukaryotic host cells such as yeast and filamentous fungi producing human-like glycoproteins characterized as having a terminal β-galactose residue and essentially lacking fucose and sialic acid residues. Further disclosed is a method for catalyzing the transfer of a galactose residue from UDP-galactose onto an acceptor substrate in a recombinant eukaryotic host cell, which can be used as a therapeutic glycoprotein.

U.S. Pat. No. 8,986,949 discloses genetically engineered strains of non-mammalian eukaryotes expressing catalytically active endomannosidase genes to enhance the processing of the N-linked glycan structures with the overall goal of obtaining a more human-like glycan pattern. In addition, cloning and expression of a novel human and mouse endomannosidase are disclosed.

U.S. Pat. No. 9,359,628 discloses genetically engineered strains of Pichia capable of producing proteins with smaller glycans. In particular, the genetically engineered strains are capable of expressing either or both of an α-1,2-mannosidase and glucosidase II. The genetically engineered strains can be further modified such that the OCH1 gene is disrupted. Methods of producing glycoproteins with smaller glycans using such genetically engineered stains of Pichia are also provided.

U.S. Pat. No. 9,695,454 discloses compositions including filamentous fungal cells, such as Trichoderma fungal cells, having reduced protease activity and expressing fucosylation pathway. Further described are methods for producing a glycoprotein having fucosylated N-glycan, using genetically modified filamentous fungal cells, for example, Trichoderma fungal cells, as the expression system.

Thermothelomyces heterothallica(Th. heterothallica) strain C1 (recently renamed from Myceliophthora thermophila, which was renamed from Chrysosporium lucknowense) is a thermo-tolerant ascomycetous filamentous fungus producing high levels of cellulases, which made it attractive for production of these and other enzymes on a commercial scale.

For example, U.S. Pat. No. 8,268,585 and 8,871,493 disclose a transformation system in the field of filamentous fungal hosts for expressing and secreting heterologous proteins or polypeptides. Also disclosed is a process for producing large amounts of polypeptide or protein in an economical manner. The system comprises a transformed or transfected fungal strain of the genus Chrysosporium,more particularly of Chrysosporium lucknowense and mutants or derivatives thereof. Also disclosed are transformants containing Chrysosporium coding sequences, as well expression-regulating sequences of Chrysosporium genes.

Wild type C1 was deposited in accordance with the Budapest Treaty with the number VKM F-3500 D, deposit date Aug. 29, 1996. High Cellulase (HC) and Low Cellulase (LC) strains have also been deposited, as described, for example, in U.S. Pat. No. 8,268,585. For example: strain UV13-6, deposit no. VKM F-3632 D, strain NG7C-19, deposit no. VKM F-3633 D, strain UV18-25, deposit no. VKM F-3631 D.

Additional improved C1 strains that have been deposited include (i) HC strain UV18-100f (Δalp1Δpyr5)—deposit no. CBS141147; (ii) HC strain UV18-100f (Δalp1Δpep4Δalp2Δpyr5Δprt1) deposit no. CBS141143; (iii) LC strain W1L #1001(Δchi1Δalp1Δalp2Δpyr5)—deposit no. CBS141153; and (iv) LC strain W1L #1001(Δchi1Δalp1Δpyr5)—deposit no. CBS141149.

EP 2505651 discloses an isolated fungus that has been mutated or selected to have low protease activity, wherein the fungus has less than 50% of the protease activity as compared to a non-mutated fungus. The fungus is of the genus Chrysosporium, preferably it is a strain of Chrysosporium lucknowense.

WO 2021/094935 to the Applicant of the present invention discloses genetically-modified Thermothelomyces heterothallica in which protein glycosylation pathways have been engineered to produce proteins with N-glycans similar to those of human. WO 2021/094935 discloses deletion or disruption of the alg3 and alg11 gene, over-expression of flippase and expression of heterologous GlcNAc transferase 1 (GNT1) and GlcNAc transferase 2 (GNT2).

There is a need for additional, alternative expression systems for producing recombinant human, companion animal and other mammalian proteins that are able to produce high yields of glycoproteins with N-glycans of mammalian proteins, particularly human and companion animal proteins, such that the proteins are suitable for therapeutic use in humans, companion animals and other mammals.

### SUMMARY OF THE INVENTION

The present invention provides Thermothelomyces heterothallica genetically modified to produce glycoproteins with N-glycans of mammalian proteins, particularly N-glycans of human proteins. The genetic modification of the Th. heterothallica of the present invention comprises deletion or disruption of the alg3 gene, heterologous expression or overexpression of ER-targeted Mannosidase 1 (alpha-1,2-Mannosidase) and ER-targeted Glucosidase 2 alpha-subunit. The genetic modification of the Th. heterothallica may also further comprise expression of heterologous GlcNAc transferase 1 (GNT1) and GlcNAc transferase 2 (GNT2). In some embodiments, the genetic modification further comprises expression of the STT3 subunit of a heterologous oligosaccharyltransferase (OST). In additional embodiments, the genetic modification further comprises expression of a heterologous galactosyltransferase. In some embodiments, the genetic modification further comprises over-expression of an endogenous flippase or expression of a heterologous flippasc.

The present invention is based in part on the finding that Th. heterothallica genetically-modified as disclosed herein produces glycoproteins in which the desired mammalian/human N-glycans constitute over 90% of the N-glycans found on the glycoproteins, and in some cases even over 95%, or over 98% of the N-glycans. In addition, when further modified to express a heterologous mammalian glycoprotein (e.g., an antibody), the Th. heterothallica genetically-modified as disclosed herein produces high levels of the heterologous glycoprotein, with the desired mammalian/human N-glycans constituting over 90% of its N-glycans. This is in contrast to hitherto described expression systems, which produce large variation in the obtained N-glycans. Remarkably, no major negative effects on cell viability have been observed with any of the modifications done.

It is further disclosed that expression of ER-targeted Mannosidase 1 (alpha-1,2-Mannosidase) and ER-targeted Glucosidase 2 alpha-subunit does not deteriorate the cell viability, or the production levels of the heterologous glycoprotein. It is now disclosed, unexpectedly, that the desired mammalian/human N-glycans in the produced glycoprotein was achieved without the need to disrupt the expression of endogenous Alpha-1,2-Mannosyltransferase (ALG11).

It is noted that Th. heterothallica, unlike most fungi and yeast, does not have hypermannosylated N-glycans, which may contain up to 50 mannose residues, but rather has “oligo mannose” N-glycans, containing between 3-9 mannose residues, and hybrid type N-glycans, containing mannose and HexNAc residues, whose structure is not fully characterized. Since the structure, as well as the synthesis pathway, of the hybrid N-glycans is not fully characterized, it was unclear that such glycans can be eliminated using the genetic modifications described herein. Surprisingly, the genetic modifications according to the present invention suffice to result in essential elimination of these structures, with over 90% of the N-glycoforms being the desired mammalian/human N-glycans, without the need to reduce the expression of alg11.

Advantageously, the Th. heterothallica cells of the present invention produce high yields of proteins. The protein levels obtained using the Th. heterothallica cells of the present invention are much higher than those obtained using, for example, yeasts.

The present invention therefore provides an efficient system for producing glycoproteins with desired N-glycans, suitable for therapeutic use in humans.

According to one aspect, the present invention provides a Thermothelomyces heterothallica genetically modified to produce glycoproteins with mammalian N-glycans, wherein the genetic modification comprises:
(i) deletion or disruption of the alg3 gene such that the Th. heterothallica fails to produce a functional alpha-1,3-mannosyltransferase;
(ii) expression of ER-targeted Mannosidase 1 (alpha-1,2-Mannosidase); and
(iii) expression of ER-targeted Glucosidase 2 alpha-subunit.

In some embodiments, the Mannosidase 1 is Trichoderma reesei mannosidase 1. In other embodiments, the Mannosidase 1 is Th. heterothallica mannosidase 1. In some embodiments, the Glucosidase 2 alpha-subunit is selected from the group consisting of Th. heterothallica, T. reesei and Aspergillus niger Glucosidase 2 alpha-subunit. In additional embodiments, the genetic modification further comprising expression of Glucosidase beta-subunit.

In some embodiments, the ER-targeted Trichoderma reesei mannosidase 1 comprises the amino acid sequence set forth in SEQ ID NO: 2. In some embodiments, ER-targeted Trichoderma reesei mannosidase 1 is encoded by an exogenous polynucleotide introduced into the Th. heterothallica which comprises the sequence set forth in SEQ ID NO: 1, or an analog or derivative thereof having at least 90% sequence identity.

In some embodiments, the ER-targeted Th. heterothallica Glucosidase 2 alpha-subunit comprises the amino acid sequence set forth in SEQ ID NO: 4. In some embodiments, the ER-targeted Th. heterothallica Glucosidase 2 alpha-subunit is encoded by an exogenous polynucleotide introduced into the Th. heterothallica which comprises the sequence set forth in SEQ ID NO: 3, or an analog or derivative thereof having at least 90% sequence identity.

In some embodiments, the ER-targeted Mannosidase 1 (alpha-1,2-Mannosidase) and/or ER-targeted Glucosidase 2 alpha-subunit are integrated to the alp3 protease locus within the Th. heterothallica genome. In certain embodiments, the ER-targeted Mannosidase 1 (alpha-1,2-Mannosidase) and ER-targeted Glucosidase 2 alpha-subunit are both integrated to the alp3 protease locus within the Th. heterothallica genome.

In some embodiments, the genetic modification further comprises expression of heterologous GlcNAc transferase 1 (GNT1) and GlcNAc transferase 2 (GNT2).

In some embodiments, heterologous GNT1 and GNT2 according to the present invention are animal-derived.

In some embodiments, an animal-derived GNT1 according to the present invention comprises a heterologous Golgi localization signal.

In some embodiments, an animal-derived GNT1 according to the present invention is human GNT1. In some embodiments, an animal-derived GNT1 according to the present invention is human GNT1 comprising a heterologous Golgi localization signal.

In some embodiments, an animal-derived GNT1 according to the present invention is bovine GNT1. In some embodiments, an animal-derived GNT1 according to the present invention is bovine GNT1 comprising a heterologous Golgi localization signal.

In some embodiments, a heterologous Golgi localization signal according to the present invention is a Th. heterothallica Golgi localization signal. In some embodiments, the Th. heterothallica Golgi localization signal is from the Th. heterothallica protein KRE2.

In other embodiments, a heterologous Golgi localization signal according to the present invention is a yeast Golgi localization signal. In some embodiments, the yeast Golgi localization signal is from the yeast protein KRE2.

In some embodiments, an animal-derived GNT2 according to the present invention is rat GNT2. In other embodiments, an animal-derived GNT2 according to the present invention is human GNT2.

In some embodiments, the animal-derived GNT1 is human GNT1 and the animal-derived GNT2 is rat GNT2. In some embodiments, the human GNT1 comprises a Th. heterothallica Golgi-localization signal. In some embodiments, the Th. heterothallica Golgi localization signal is from the C1 Th. heterothallica protein KRE2. In other embodiments, the human GNT1 comprises a yeast Golgi localization signal.

In some embodiments, the animal-derived GNT1 is human GNT1 and the animal-derived GNT2 is rat GNT2. In some embodiments, the human GNT1 comprises a Th. heterothallica Golgi-localization signal. In some embodiments, the Th. heterothallica Golgi localization signal is from the C1 protein KRE2. In other embodiments, the human GNT1comprises a yeast Golgi localization signal.

In some embodiments, the Th. heterothallica according to the present invention is genetically modified to overexpress the endogenous Th. heterothallica RFT1 flippase.

In other embodiments, the Th. heterothallica according to the present invention is genetically modified to express a heterologous flippase, wherein the heterologous flippase is the yeast FLC2p flippase.

In some embodiments, the genetic modification according to the present invention further comprises expression of the STT3 subunit of a heterologous oligosaccharyltransferase (heterologous STT3). In some embodiments, a heterologous STT3 according to the present invention is Leishmania STT3.

In some embodiments, the genetic modification according to the present invention further comprises expression of a heterologous galactosyltransferase. In some embodiments, the heterologous galactosyltransferase is an animal-derived galactosyltransferase. In some embodiments, the animal-derived galactosyltransferase is a human galactosyltransferase. In some embodiments, an animal-derived galactosyltransferase according to the present invention is a human galactosyltransferase comprising a heterologous Golgi localization signal, for example, comprising the Th. heterothallica KRE2 Golgi-localization signal.

In additional embodiments, the animal-derived galactosyltransferase is a Xenopus tropicalis galactosyltransferase. In some embodiments, an animal-derived galactosyltransferase according to the present invention is a Xenopus tropicalis galactosyltransferase comprising a heterologous Golgi localization signal, for example, comprising the S. cerevisiae KRE2 Golgi-localization signal.

In some embodiments, the Th. heterothallica is Th. heterothallica C1.

In some embodiments, the C1 is a strain modified to delete one or more genes encoding an endogenous protease.

In some embodiments, the C1 is a strain modified to delete a gene encoding an endogenous chitinase.

In some embodiments, the C1 is a strain selected from the group consisting of: wild type C1 deposit no. VKM F-3500 D, UV13-6 deposit no. VKM F-3632 D, NG7C-19 deposit no. VKM F-3633 D, UV18-25, deposit no. VKM F-3631 D, W1L #100I (prt-Δalp1Δchi1Δalp2Δpyr5) deposit no. CBS141153, UV18-100f (prt-Δalp1, Δpyr5) deposit no. CBS141147, W1L #100I (prt-Δalp1Δchi1Δpyr5) deposit no. CBS141149, and UV18-100f (prt-Δalp1Δpep4Δalp2Δprt1Δpyr5) deposit no. CBS141143. Each possibility represents a separate embodiment of the present invention. According to certain embodiments, the C1 strain has reduced expression and/or activity of at least 8, at least 9, at least 10, at least 11, at least 12, at least 13, at least 14, or more proteases. According to certain exemplary embodiments, the C1 strain has reduced expression or activity of ALP1, ALP2, PEP4, PRT1, SRP1, ALP3, PEP1, and MTP2 (ΔalpΔalp2Δpep4Δprt1Δsrp1Δalp3Δpep1Δmtp2).

According to some embodiments, the Th. heterothallica is capable of producing a heterologous mammalian glycoprotein having mammalian/human N-glycans constituting over 90%, 95% or 98% of the N-glycans found on said glycoprotein.

In some embodiments, the Th. heterothallica is further genetically modified to express a heterologous mammalian glycoprotein. In some embodiments, the heterologous mammalian glycoprotein is an antibody or an antigen-binding fragment thereof.

According to some embodiments, the heterologous mammalian glycoprotein comprises mammalian/human N-glycans which constitute over 90% of its total N-glycans. According to some embodiments, the heterologous mammalian glycoprotein comprises mammalian/human N-glycans which constitute over 92%, 94%, 96%, 98% or 99% of its total N-glycans.

According to some embodiments, the heterologous mammalian glycoprotein comprises an amount of mammalian/human N-glycans which is at least 80%. 85%, 90%, 95% or more of the amount of N-glycans found in same glycoprotein produced by mammalian/human cells.

According to another aspect, the present invention provides a method for generating a Th. heterothallica that produces glycoproteins with mammalian N-glycans, comprising:
(a) deleting or disrupting the alg3 gene of the Th. heterothallica such that the . heterothallica fails to produce a functional alpha-1,3-mannosyltransferase;
(b) introducing into the Th. heterothallica an exogenous polynucleotide encoding ER-targeted Mannosidase 1 (alpha-1,2-Mannosidase); and
(c) introducing into the Th. heterothallica an exogenous polynucleotide encoding ER-targeted Glucosidase 2 alpha-subunit.

According to some embodiments, the method further comprising a step of introducing into the Th. Heterothallica: an exogenous polynucleotide encoding an endogenous flippase to induce over-expression of said endogenous flippase in the Th. Heterothallica; or an exogenous polynucleotide encoding a heterologous flippase to induce expression of said heterologous flippase in the Th. Heterothallica.

According to some embodiments, the ER-targeted Mannosidase 1 (alpha-1,2-Mannosidase) and ER-targeted Glucosidase 2 alpha-subunit are introduced using a single exogenous polynucleotide encoding both enzymes.

In some embodiments, the Mannosidase 1 is Trichoderma reesei mannosidase 1. In some embodiments, the Glucosidase 2 alpha-subunit is Th. heterothallica Glucosidase 2 alpha-subunit.

According to a further aspect, the present invention provides a method for producing a glycoprotein with mammalian N-glycans, the method comprising:
(a) providing a Th. heterothallica genetically modified according to the present invention;
(b) culturing the Th. heterothallica under conditions suitable for expressing the glycoprotein; and
(c) recovering the glycoprotein.

In some embodiments, the glycoprotein is a heterologous mammalian glycoprotein recombinantly expressed in the Th. heterothallica. In some particular embodiments, the glycoprotein is a human protein recombinantly expressed in the Th. heterothallica. In other embodiments, the glycoprotein is a protein of a companion animal recombinantly expressed in the Th. heterothallica. In some embodiments, the heterologous mammalian glycoprotein is an antibody or an antigen-binding fragment thereof.

According to some embodiments, the heterologous mammalian glycoprotein comprises mammalian/human N-glycans which constitute over 90%, 92%, 94%, 96%, 98% or 99% of its total N-glycans. According to additional embodiments, the heterologous mammalian glycoprotein comprises an amount of mammalian/human N-glycans which is at least 80%. 85%, 90%, 95% or more of the amount of N-glycans found in same glycoprotein produced by mammalian/human cells.

According to a further aspect, the present invention provides a recombinant glycoprotein produced by the Th. heterothallica genetically modified according to the present invention, wherein the glycoprotein comprises GlcNAc₂Man₃GlcNAc₂ (G0) glycans.

According to a further aspect, the present invention provides a recombinant glycoprotein produced by the Th. heterothallica genetically modified according to the present invention, wherein the glycoprotein comprises Gal₁GlcNAc₂Man₃GlcNAc₂ (G1) glycans, Gal₂GlcNAc₂Man₃GlcNAc₂ (G2) glycans or a combination thereof.

In some embodiments, the recombinant glycoprotein produced by the Th. heterothallica genetically modified according to the present invention is a pharmaceutical grade glycoprotein.

According to some embodiments, the heterologous mammalian glycoprotein comprises mammalian/human N-glycans which constitute over 90%, 92%, 94%, 96%, 98% or 99% of its total N-glycans. According to additional embodiments, the heterologous mammalian glycoprotein comprises an amount of mammalian/human N-glycans which is at least 80%. 85%, 90%, 95% or more of the amount of N-glycans found in same glycoprotein produced by mammalian/human cells.

It is to be understood that any combination of each of the aspects and the embodiments disclosed herein is explicitly encompassed within the disclosure of the present invention.

These and further aspects and features of the present invention will become apparent from the detailed description, examples and claims which follow.

### BRIEF DESCRIPTION OF THE FIGURES

FIGS. 1A-1D. Analysis of released N-glycans of Protein A affinity purified Nivolumab from glycomodified strains. One chromatogram from each transformation is shown as an example. Results for main N-glycans for all strains described in the text are shown in the corresponding tables below the chromatograms. FIG. 1A—Strains M4855 and M4856 cultivated in shake flasks (M3291 based strains with T. reesei Mns1-HDEL and C1 gls2a-HDEL expression). Chromatogram of M4855 and a table of main released N-glycans for both M4855 and M4856 are shown. FIGS. 1B-1D—G1/2 strains M5129 to M5132 cultivated in shake flasks (M4855 based strains with expression of G1/2 machinery). Chromatograms of M5130 (FIG. 1B) and M5132 (FIG. 1C) and a table of main released N-glycans for M5129 to M5132 (FIG. 1D) are shown.

FIG. 2. Comparison of the amount of released N-glycans of Nivolumab produced by strains M5129 to M5132 cultivated in shake flasks (M4855 based strains with expression of G1/2 machinery). Amount of released N-glycans have been normalized between samples using a fixed amount of internal standard added to each sample for the analysis. Response % is set to 100% for reference protein Opdivo.

FIGS. 3A-3D. Analysis of released N-glycans of Protein A affinity purified Nivolumab from fermentation samples of three glycomodified strains. Results for main N-glycans for all strains described in the text are shown in the corresponding tables below the chromatograms. FIG. 3A—Starting strain M3291 with alg3 deletion only. Chromatogram of M3291 and a table of main released N-glycans from fermentation conditions are shown. FIGS. 3B-3D—G1/2 strains M5130 and M5132. Chromatograms of M5130 (FIG. 3B) and M5132 (FIG. 3C) and a table of main released N-glycans from fermentation conditions (FIG. 3D) are shown.

FIG. 4. Comparison of the amount of released N-glycans of Nivolumab produced by strains M5130 and M5132 cultivated in fermentation conditions. Amount of released N-glycans have been normalized between samples using a fixed amount of internal standard added to each sample for the analysis. Response % is set to 100% for reference protein Opdivo.

FIGS. 5A-5C. Analysis of released N-glycans of total secreted proteins from glycomodified strains. One chromatogram from each transformation is shown as an example. Results for main N-glycans for all strains described in the text are shown in the table (FIG. 5C) and the chromatograms (FIGS. 5A-5B). M6589 and M6596 are strains with G1/2 machinery where TrMns1-HDEL is under ubiquitin-like protein promoter whereas M6590 and M6597 are strains with G1/2 machinery where TrMns1-HDEL is under bg18 promoter.

### DETAILED DESCRIPTION OF THE INVENTION

The present invention is directed to genetic modification of the fungus Thermothelomyces heterothallica, particularly the strain C1, to produce glycoproteins with N-glycans of mammalian proteins, particularly N-glycans of human, companion animal and other mammalian proteins.

The glycoproteins produced by Th. heterothallica genetically-modified as described herein are suitable for therapeutic use in humans, companion animals such as dogs, cats and horses, and other mammals.

Protein glycosylation, namely, the covalent attachment of oligosaccharides to side chains of newly synthesized polypeptide chains in cells, is an ordered process in eukaryotic cells involving a series of enzymes that sequentially add and remove saccharide moieties. N-glycosylation is the process in which an oligosaccharide is attached to the side chain of an asparagine residue, particularly an asparagine which occurs in the sequence Asn-Xaa-Ser/Thr, where Xaa represents any amino acid except Pro.

N-glycosylation initiates in the endoplasmic reticulum (ER), where the oligosaccharide Glc₃Man₉GlcNAc₂ is assembled on a lipid carrier, dolichol-pyrophosphate. and subsequently transferred to selected asparagine residues of polypeptides that have entered the lumen of the ER. The biosynthesis of the lipid-linked oligosaccharide requires the activity of several specific glycosyltransferases (e.g., ALG1, ALG2, and ALG3). It begins at the cytoplasmic side of the ER membrane and terminates in the lumen where oligosaccharyltransferase (OST) selects N-X-S/T sequons of a nascent polypeptide and generates the N-glycosidic linkage between the side chain amide of asparagine and the oligosaccharide. The flipping of the lipid-linked oligosaccharide from outside the ER to the inside is carried out by a flippase located at the ER membrane. Following transfer to the nascent polypeptide, the oligosaccharide is typically trimmed by glucosidases and mannosidases and the nascent glycoprotein is then transferred to the Golgi apparatus for further processing.

The synthesis of the dolichol pyrophosphate-bound oligosaccharide is essentially conserved in all known eukaryotes. However, further processing of the oligosaccharide as the glycoprotein moves along the secretory pathway varies greatly between lower eukaryotes such as fungi or yeasts and higher eukaryotes such as animals and plants. Thus, the final composition of a sugar side chain is different between various organisms, and depends upon the host.

In microorganisms such as yeasts, typically additional mannose and/or mannosylphosphate sugars are added, resulting in “high-mannose” type N-glycans which may contain up to 30-50 mannose residues.

In animal cells, including human, companion animal and other mammalian cells, the nascent glycoprotein is transferred to the Golgi apparatus where mannose residues are removed by Golgi-specific 1,2-mannosidases. Processing continues as the protein proceeds through the Golgi by a number of modifying enzymes including N-acetylglucosamine transferases (GnT I, GnT II, GnT III, GnT IV, GnT V, GnT VI), mannosidase II and fucosyltransferases that add and remove specific sugar residues. Finally, the N-glycans are acted on by galactosyl transferases (GalT) and sialyltransferases (ST) and the finished glycoprotein is released from the Golgi apparatus. The N-glycans of animal glycoproteins have bi-, tri-, or tetra-antennary structures, and may typically include galactose, fucose and N-acetylglucosamine. Commonly the terminal residues of the N-glycans consist of sialic acid.

Th. heterothallica, unlike most fungi and yeast, does not have hypermannosylated N-glycans, but rather has “oligo mannose” glycans—Man₃ to Man₈₋₉—and hybrid type glycans containing both Man and HexNAc residues (Man₃HexNac-Man₈HexNac). The exact structure of these hybrid glycans is not completely known. The hybrid glycans have the typical mannose residues but in addition an unknown HexNAc attached via a yet uncharacterized bond.

Since the structure, as well as the synthesis pathway, of the hybrid glycans is not fully characterized, it was unclear that such glycans can be eliminated using the genetic modifications described herein. Surprisingly, the genetic modification according to the present invention resulted in essential elimination of these structures, with over 90%, and often over 98% of the N-glycoforms being the desired mammalian/human glycans.

The present invention is directed to genetic modification of the N-glycosylation pathway in Th. heterothallica such that it produces high percentage of glycoproteins with mammalian N-glycans, particularly human N-glycans, such as GlcNAc₂Man₃GlcNAc₂ (“G0”), GlcNAc₂Man₃GlcNAc₂(Fuc) (“FG0”), Gal₁₋₂GlcNAc₂Man₃GlcNAc₂(“G1”/“G2”) and Gal₁₋₂GlcNAc₂Man₃GlcNAc₂(Fuc) (“FG1”/“FG2”).

In particular, in some embodiments, the genetic modification of the N-glycosylation pathway in Th. heterothallica comprises the following:
1. Deletion of the C1 alg3 gene (encoding alpha-1,3-mannosyltransferase);
2. expression of ER-targeted Trichoderma reesei Mannosidase 1 (alpha-1,2-Mannosidase);
3. expression of ER-targeted C1 Glucosidase 2 alpha-subunit;
4. Expression of a heterologous GlcNAc transferase 1 (GNT1)
5. Expression of a heterologous GlcNAc transferase 2 (GNT2); and
6. Expression of a heterologous Galactosyltransferase 1 (GalT1).

The deletion of alg3 terminates the synthesis of the N-glycan precursor at Man₅GlcNAc₂ with 1 or 2 terminal glucoses This glycan serves as the substrate for GNT1and GNT2 that are introduced to the Th. heterothallica. Additional genetic modifications may include introduction of additional enzymes from the human, companion animal and other mammalian glycosylation pathways, such as galactosyltransferase and/or fucosyltransferase.

The above-described heterologous enzymes are expressed with targeting peptides, such that the expressed enzymes are targeted to specific cell compartments.

As used herein, when an enzyme is mentioned, it encompasses enzymatically-active fragments thereof and enzymatically-active variants thereof.

The present invention is particularly directed to engineering of the N-glycosylation pathway of Th. heterothallica. It is noted that O-glycans may be present or removed or altered by further genetic modifications of the Th. heterothallica.

It is to be understood that the genetic modifications according to the present invention are such that the genetically-modified Th. heterothallica is able to grow at sufficient rates suitable for its intended use.

As used herein “C1” or “Thermothelomyces heterothallica C1” or “Th. heterothallica C1”, all refer to Thermothelomyces heterothallica strain C1. Description of the genus Thermothelomyces and its species can be found, for example, in Marin-Felix Y (2015. Mycologica 107 (3): 619-632) and van den Brink J et al. (2012, Fungal Diversity 52 (1): 197-207).

It is noted that the above authors (Marin-Felix et al., 2015) proposed splitting of the genus Myceliophthora based on differences in optimal growth temperature, morphology of the conidiospore, and details of the sexual reproduction cycle. According to the proposed criteria C1 clearly belongs to the newly established genus Thermothelomyces, which contain former thermotolerant Myceliophthora species rather than to the genus Myceliophthora, which remains to include the non-thermotolerant species. As C1 can form ascospores with some other Thermothelomyces (formerly Myceliophthora) strains with opposite mating type, C1 is best classified as Th. heterothallica strain C1, rather than Th. thermophila C1.

It must also be appreciated that the fungal taxonomy was also in constant move in the past, so the current names listed above may be preceded by a variety of older names beyond Myceliophthora thermophila (van Oorschot, 1977. Persoonia 9 (3): 403), which are now considered synonyms. For example, Thermothelomyces heterothallica (Marin-Felix et al., 2015. Mycologica, 3: 619-63), is synonymized with Corynascus heterotchallica (von Arx et al., 1983), Thermothelomyces heterothallica (von Klopotek, 1976. Archives of Microbiology 107 (2), 223-224), Chrysosporium lucknowense and thermophile (von Klopotek, 1974. Archives of Microbiology 98 (1), 365-369) as well as Sporotrichium thermophile (Alpinis 1963. Nova Hedwigia 5: 74).

It is further to be explicitly understood that the present invention encompasses any strain containing a ribosomal DNA (rDNA) sequence that shows 99% homology or more to SEQ ID NO: 22, and all those strains are considered to be conspecific with Thermothelomyces heterothallica.

SEQ ID NO: 22 is 99.98% identical with the rDNA sequence found on chromosome 7 of Th. heterothallica/thermophila (listed as Myceliophtora thermophilica) ATCC 42464 rDNA sequence (ncbi.nlm.nih.gov/nucleotide/CP003008.1). Th. heterothallica strain C1 (as Chrysosporium lucknowense strain C1) was deposited in accordance with the Budapest Treaty with the number VKM F-3500 D, deposit date Aug. 29, 1996.

The above terms also encompass genetically modified sub-strains derived from the wild type strain, which have been mutated, using random or directed approaches, for example, using UV mutagenesis, or by deleting one or more endogenous genes. For example, the C1 strain may refer to a wild type strain modified to delete one or more endogenous genes encoding an endogenous protease and/or one or more genes encoding an endogenous chitinase. For example, C1 strains (sub-strains) which are encompassed by the present invention include UV18-25, deposit No. VKM F-3631 D; strain NG7C-19, deposit No. VKM F-3633 D; and strain UV13-6, deposit No. VKM F-3632 D. Further C1 strain that may be used according to the teachings of the present invention include HC strain UV18-100f deposit No. CBS141147; HC strain UV18-100f deposit No. CBS141143; LC strain W1L #100I deposit No. CBS141153; and LC strain W1L #100I deposit No. CBS141149. Th. heterothallica fungi in general and strain C1 in particular show higher biomass production compared to yeast strains when grown in suitable conditions. Th. heterothallica fungi can grow in large volumes of 3 dimensions (3D) liquid cultures as well as on solid medium. Several strains developed by the Applicant of the present invention are less sensitive to feedback repression by glucose and other fermentable sugars present in the fungal growth medium as carbon source compared to conventional yeast and other fungi, and can tolerate high feeding rate of the carbon source leading to high yields. Furthermore, some of these strains provide significantly reduced medium viscosity when grown in commercial fermenters compared to the high viscosity obtained with non-glucose repressed wild type Th. heterothallica fungi or with other filamentous fungi known to be used for proteins production. The low viscosity may be attributed to the morphological change of the strain from having long and highly interlaced hyphae in the parental strain(s) to short and less interlaced hyphae in the developed strain(s). Low medium viscosity is highly advantageous in large scale industrial production in fermenters. For example, the Th. heterothallica C1 strain UV18-25, deposit No. VKM F-3631 D, which shows reduced sensitivity to glucose repression, has been grown industrially to produce recombinant enzymes at volumes of more than 100,000 liters.

In some embodiments, the C1 strain of the present invention is a strain modified to delete a plurality (i.e., at least two) genes encoding endogenous proteases. In some embodiments, the C1 strain is a strain modified to delete at least four genes encoding endogenous proteases. In additional embodiments, the C1 strain is a strain modified to delete at least five genes encoding endogenous proteases. In some particular embodiments, the C1 strain is a strain modified to delete at least six genes encoding endogenous proteases. In additional particular embodiments, the C1 strain is a strain modified to delete at least eight genes encoding endogenous proteases. In additional particular embodiments, the C1 strain is a strain modified to delete at least 8, 9, 10, 11, 12, 13, 14 or more genes encoding endogenous proteases. In certain exemplary embodiments, the C1 strain is a strain modified to delete at least 13 or 14 genes encoding endogenous proteases.

It is to be explicitly understood that the teachings of the present invention encompass mutants, derivatives, progeny, clones and analogous of the Th. heterothallica C1 strains, as long as these derivatives, progeny, clones and analogous, when genetically modified according to the teachings of the present invention, are capable of growing and producing a protein with N-glycans as described herein.

It is to be explicitly understood that the term “derivative” with reference to fungal line encompasses any fungal parent line with modifications positively affecting product yield, efficiency, or efficacy, or affecting any trait improving the fungal derivative as a tool to produce heterologous proteins with N-glycans of mammalian proteins, particularly of human, companion animals and other mammalian proteins, as described herein. As used herein, the term “progeny” refers to an unmodified descendant from the parent fungal line, such as cell from cell.

As used herein, “glycan” refers to an oligosaccharide chain that can be linked to a carrier such as an amino acid, peptide, polypeptide, lipid or a reducing end conjugate. The present invention particularly relates to N-linked glycans (“N-glycan”) conjugated to a polypeptide N-glycosylation site such as -Asn-Xxx-Ser/Thr- by N-linkage to side-chain amide nitrogen of asparagine residue (Asn), where Xxx is any amino acid residue except Pro. The present invention may further relate to glycans as part of dolichol-phospho-oligosaccharide (Dol-P-P-OS) precursor lipid structures, which are precursors of N-linked glycans in the endoplasmic reticulum of eukaryotic cells. The precursor oligosaccharides are bound by their reducing end to two phosphate residues on the dolichol lipid.

The monosaccharides typically constituting N-glycans found in mammalian glycoproteins, include, without limitation, N-acetylglucosamine (abbreviated “GlcNAc”), mannose (abbreviated “Man”), glucose (abbreviated “Glc”), galactose (abbreviated “Gal”), sialic acid (abbreviated “Neu5Ac”) and fucose (abbreviated “Fuc”).

N-glycans share a common pentasaccharide referred as the “core” structure Man₃GlcNAc₂ (abbreviated “Man₃”). Important target glycan structures of the present invention include N-glycans which have one GlcNAc residue on the terminal 1,3 mannose arm of the core structure and one GlcNAc residue on the terminal 1,6 mannose arm of the core structure. Such N-glycans include: GlcNAc₂Man₃GlcNAc₂ (termed “G0” glycoform), Gal₁₋₂GlcNAc₂Man₃GlcNAc₂ (termed “G1” or “G2” glycoform according to the number of galactose residues), and their core fucosylated glycoforms: GlcNAc₂Man₃GlcNAc₂(Fuc) (“G0F” or “FG0”) and Gal₁₋₂GlcNAc₂Man₃GlcNAc₂(Fuc) (“G1F” and “G2F”, or “FG1” and “FG2”).

The term “alg3 gene” refers to the gene encoding alpha-1,3-mannosyltransferase. The term “alpha-1,3-mannosyltransferase” refers to dolichyl-P-Man: Man₅GlcNAc₂-PP-dolichol alpha-1,3-mannosyltransferase (EC 2.4.1.258), which is an ER-resident enzyme that catalyzes the reaction:

dolichyl beta-D-mannosyl phosphate+D-Man-alpha-(1->2)-D-Man-alpha-(1->2)-D-Man-alpha-(1->3)-[D-Man-alpha-(1->6)]-D-Man-beta-(1->4)-D-GlcNAc-beta-(1->4)-D-GlcNAc-diphosphodolichol

→

D-Man-alpha-(1->2)-D-Man-alpha-(1->2)-D-Man-alpha-(1->3)-[D-Man-alpha-(1->3)-D-Man-alpha-(1->6)]-D-Man-beta-(1->4)-D-GlcNAc-beta-(1->4)-D-GlcNAc-diphosphodolichol+dolichyl phosphate

In some particular embodiments, “alg3 gene” is the gene encoding alpha-1,3-mannosyltransferase of C1 (ortholog of JGI M. thermophila genome (mycocosm.jgi.doe.gov) accession no. 2310419).

The Th. heterothallica of the present invention is genetically modified by deletion or disruption of the alg3 gene such that the Th. heterothallica fails to produce a functional alpha-1,3-mannosyltransferase. The Th. heterothallica of the present invention does not display a detectable alpha-1,3-mannosyltransferase activity.

The term “Mannosidase 1” (alpha-1,2-Mannosidase), abbreviated “MDS1” or “MNS1”, catalyzes the reaction:

3xH₂O+N4-(α-D-Man-(1→2)-α-D-Man-(1→2)-α-D-Man-(1→3)-[α-D-Man-(1→3)-[α-D-Man-(1→2)-α-D-Man-(1→6)]-α-D-Man-(1→6)]-β-D-Man-(1→4)-β-D-GlcNAc-(1→4)-α-D-GlcNAc)-L-asparaginyl-[protein] (N-glucan mannose isomer 8A1,2,3B1,3)→3 β-D-mannose+N4-(α-D-Man-(1→3)-[α-D-Man-(1→3)-[α-D-Man-(1→6)]-α-D-Man-(1→6)]-β-D-Man-(1→4)-β-D-GlcNAc-(1→4)-β-D-GlcNAc)-L-asparaginyl-[protein] (N-glucan mannose isomer 5A1,2).

An exemplified accession number of alpha-1,2-Mannosidase is Uniprot Q9P8T8.

The term “Glucosidase 2 alpha-subunit” (GLS2-alpha or GLS2α) refers to an enzyme that cleaves sequentially the 2 innermost alpha-1,3-linked glucose residues from the Glc2Man9GlcNAc2 oligosaccharide precursor of immature glycoproteins.

The term “flippase” (EC 7.6.2.1) refers to an enzyme that transfers the lipid-linked glycan precursor during its synthesis in the ER from the cytosolic side to the luminal side of the ER. The term “GlcNAc transferase 1”, abbreviated “GNT1” (also “GnTI”), refers to alpha-1,3-mannosyl-glycoprotein 2-beta-N-acetylglucosaminyltransferase (EC 2.4.1.101), which is a Golgi-resident enzyme that transfers a GlcNAc residue from UDP-GlcNAc to the acceptor substrate Man₅GlcNAc₂, to produce GlcNAcMan₅GlcNAc₂. In the present invention the synthesis of the N-glycan precursor generates Man3GlcNAc2 in view of the deletion of alg3 and the expression of alpha-1,2-Mannosidase and Glucosidase 2 alpha-subunit, therefore the glycan Man3GlcNAc2 serves as the substrate for GNT1, to produce GlcNAcMan3GlcNAc2.

The term “GlcNAc transferase 2”, abbreviated “GNT2” (also “GnTII”), refers to alpha-1,6-mannosyl-glycoprotein 2-beta-N-acetylglucosaminyltransferase (EC 2.4.1.143), which is a Golgi-resident enzyme that transfers a GlcNAc residue from UDP-GlcNAc to the free terminal mannose residue in GlcNAcMan₃GlcNAc₂, to produce GlcNAc₂Man₃GlcNAc₂.

The terms “STT3 subunit of oligosaccharyltransferase”, “STT3 protein” or simply “STT3” are used herein interchangeably to refer to dolichyl-diphosphooligosaccharide-protein glycosyltransferase subunit (EC: 2.4.99.18). It is the catalytic subunit of the oligosaccharyltransferase (OST) complex that catalyzes the initial transfer of a defined glycan (Glc₃Man₉GlcNAc₂ in eukaryotes) from the lipid carrier dolichol-pyrophosphate to an asparagine residue within an Asn-X-Ser/Thr consensus motif in nascent polypeptide chains, the first step in protein N-glycosylation. STT3 protein catalyzes the reaction:

Dolichyl diphosphooligosaccharide+L-asparaginyl-[protein]

→

Dolichyl diphosphate+H⁺+N⁴-(oligosaccharide-(1→4)-N-acetyl-β-D-glucosaminyl-(1→4)-N-acetyl-β-D-glucosaminyl)-L-asparaginy-[protein]

The term “galactosyltransferase” (EC 2.4.1.38) refers to a Golgi-resident enzyme that transfers β-linked galactosyl residues to terminal N-acetylglucosamine.

The term “heterologous”, when referring to a gene, enzyme, protein or peptide sequence such as a subcellular localization signal, is used herein to describe a gene, enzyme, protein or peptide sequence that is not naturally found or expressed in C1. When referring to a subcellular localization signal, the term also describes a subcellular localization signal that is different from the one naturally found in the respective protein.

The term “endogenous”, when referring to a gene, enzyme, protein or peptide sequence such as a subcellular localization signal, refers to a gene, enzyme, protein or peptide sequence that is naturally present in C1.

The term “exogenous”, when referring to a polynucleotide, is used herein to describe a synthetic polynucleotide that is exogenously introduced into the C1 via transformation. The exogenous polynucleotide may be introduced into the C1 in a stable or transient manner, so as to produce a ribonucleic acid (RNA) molecule and subsequently a polypeptide molecule.

### Expression Constructs and Vectors

The terms “expression construct”, “DNA construct” or “expression cassette” are used herein interchangeably and refer to an artificially assembled or isolated nucleic acid molecule which includes a nucleic acid sequence encoding a protein of interest and which is assembled such that the protein of interest is expressed in a target host cell. An expression construct typically comprises appropriate regulatory sequences operably linked to the nucleic acid sequence encoding the protein of interest. An expression construct may further include a nucleic acid sequence encoding a selection marker.

The terms “nucleic acid sequence”, “nucleotide sequence” and “polynucleotide” are used herein to refer to polymers of deoxyribonucleotides (DNA), ribonucleotides (RNA), and modified forms thereof in the form of a separate fragment or as a component of a larger construct. A nucleic acid sequence may be a coding sequence, i.e., a sequence that encodes for an end product in the cell, such as a protein. A nucleic acid sequence may also be a regulatory sequence, such as, for example, a promoter.

The terms “peptide”, “polypeptide” and “protein” are used herein to refer to a polymer of amino acid residues. The term “peptide” typically indicates an amino acid sequence consisting of 2 to 50 amino acids, while “protein” indicates an amino acid sequence consisting of more than 50 amino acid residues.

A sequence (such as a nucleic acid sequence and an amino acid sequence) that is “homologous” to a reference sequence refers herein to percent identity between the sequences, where the percent identity is at least 75%, preferably at least 80%, at least 85%, at least 90%, at least 95%, at least 98% or at least 99%. Each possibility represents a separate embodiment of the present invention. Homologs of the sequences described herein are encompassed within the present invention. Protein homologs are encompassed as long as they maintain the activity of the original protein. Homologous nucleic acid sequences include variations related to codon usage and degeneration of the genetic code. Sequence identity may be determined using nucleotide/amino acid sequence comparison algorithms, as known in the art.

Nucleic acid sequences encoding the polypeptides of the present invention may be optimized for expression. Examples of such sequence modifications include, but are not limited to, an altered G/C content to more closely approach that typically found in Th. heterothallica, and the removal of codons atypically found in this fungus, commonly referred to as codon optimization.

The phrase “codon optimization” refers to the selection of appropriate DNA nucleotides for use within a structural gene or fragment thereof that approaches codon usage within the organism of interest, and/or to a process of modifying a nucleic acid sequence for enhanced expression in the host cell of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in protein synthesis. Accordingly, genes can be tailored for optimal gene expression in a given organism based on codon optimization. Therefore, an optimized gene or nucleic acid sequence refers to a gene in which the nucleotide sequence of a native or naturally occurring gene has been modified in order to utilize statistically-preferred or statistically-favored codons within the organism. The present invention explicitly encompasses polynucleotides encoding the enzyme of interest as disclosed herein which are codon optimized for expression in Th. heterothallica.

The term “regulatory sequences” refer to DNA sequences which control the expression (transcription) of coding sequences, such as promoters and terminators.

The term “promoter” is directed to a regulatory DNA sequence which controls or directs the transcription of another DNA sequence in vivo or in vitro. Usually, the promoter is located in the 5′ region (that is, precedes, located upstream) of the transcribed sequence. Promoters may be derived in their entirety from a native source, or be composed of different elements derived from different promoters found in nature, or even comprise synthetic nucleotide segments. Promoters can be constitutive (i.e. promoter activation is not regulated by an inducing agent and hence rate of transcription is constant), or inducible (i.e., promoter activation is regulated by an inducing agent). In most cases the exact boundaries of regulatory sequences have not been completely defined, and in some cases cannot be completely defined, and thus DNA sequences of some variation may have identical promoter activity.

The term “terminator” is directed to another regulatory DNA sequence which regulates transcription termination. A terminator sequence is operably linked to the 3′ terminus of the nucleic acid sequence to be transcribed.

The terms “Th. heterothallica promoter” and “Th. heterothallica terminator” indicate promoter and terminator sequences suitable for use in Th. heterothallica, i.e., capable of directing gene expression in Th. heterothallica. In some particular embodiments, C1 promoters and C1 terminators are used, which indicate promoter and terminator sequences capable of directing gene expression in C1.

According to some embodiments, the Th. heterothallica promoter/terminator is derived from an endogenous gene of Th. heterothallica. According to other embodiments the Th. heterothallica promoter/terminator is derived from a gene exogenous to Th. heterothallica.

Suitable constitutive promoters and terminators include, for example, those of C1 glycolytic genes such as phosphoglycerate kinase gene (PGK) (Uniprot: G2QLD8, NCBI Reference Sequence: XM_003665967), glyceraldehyde 3-phosphate dehydrogenase (GPD) (Uniprot: G2QPQ8, NCBI Reference Sequence: XM_003666768), phosphofructokinase (PFK) (Uniprot: G2Q605, NCBI Reference Sequence: XM_003659879); or the β-glucosidase 1 gene bgl1 (Accession number: XM_003662656); or triose phosphate isomerase (TPI) (Uniprot: G2QBR0, NCBI Reference Sequence: XM_003663200); or actin (ACT) (Uniprot: G2Q7Q5, NCBI Reference Sequence: XM_003662111); or the C1 cbh1promoter (GenBank AX284115) or C1 chil promoter (GenBank HI550986). Additional promoters that can be used are Aspergillus nidulans gpdA promoter; and synthetic promoters described in Rantasalo et al. (2018 NAR 46 (18): e111). Synthetic promoters that can be used with the present invention are further described in WO 2017/144777. As exemplary terminators, the terminator of the C1 chitinase 1 gene chil (GenBank HI550986), cellobiohydrolase 1 cbh1 (GenBank AX284115) can be used, or the yeast adh1 terminator.

Exemplary promoter and terminator sequences, and promoter/terminator pairs, are provided in the Examples section that follows. In some embodiments, promoter sequences for use with the present invention are selected from the group consisting of: promoter-8, bgl8 promoter, promoter-9, promoter-3, promoter-1 and TEFIA promoter, as exemplified hereinbelow. Each possibility represents a separate embodiment of the present invention.

The term “operably linked” means that a selected nucleic acid sequence is in proximity with a regulatory element (promoter or terminator) to allow the regulatory element to regulate expression of the selected nucleic acid sequence.

The terms “localization signal”, “localization sequence”, “subcellular targeting peptide/signal/sequence” and the like are used herein interchangeably and refer to a short peptide sequence (usually 5-30 amino acids long) included within a protein sequence (typically present at one terminus of the protein such as the N-terminus) that directs the protein to a particular subcellular localization within the cell. For example, a Golgi localization signal targets the protein to the Golgi apparatus. A “heterologous localization signal”, for example, a “heterologous Golgi localization signal”, indicates a localization signal that is not the one naturally found in the protein. In some embodiments, “heterologous” refers to a localization signal from another organism.

In some embodiments, localization signals of proteins expressed in Th. heterothallica according to the present invention are derived from endogenous genes of Th. heterothallica. For example, in some embodiments, a Golgi localization signal from the C1 protein KRE2a (ortholog of JGI M. thermophila genome (mycocosm.jgi.doe.gov) accession no. 2300989) is used.

In other embodiments, localization signals of proteins expressed in Th. heterothallica according to the present invention are derived from genes exogenous to Th. heterothallica (heterologous localization signals). For example, in some embodiments, animal-derived enzymes expressed in Th. heterothallica according to the present invention are expressed with their own naturally-occurring Golgi localization signals. As another example, in some embodiments, a Golgi localization signal from yeast proteins, such as the S. cerevisiae protein KRE2 (GenBank accession no. CAA44516) is used.

According to some embodiments, the proteins expressed in Th. heterothallica comprise an ER targeting sequence. In certain embodiments, the ER targeting sequence is the sequence HDEL.

As used herein, the term “in frame”, when referring to one or more nucleic acid sequences, indicates that these sequences are linked such that their correct reading frame is preserved.

Expression constructs according to some embodiments of the present invention comprise a Th. heterothallica promoter sequence and a Th. heterothallica terminator sequence operably linked to a nucleic acid sequence encoding an enzyme, such as a flippase, GNT1 or GNT2. In some particular embodiments, expression constructs of the present invention comprise a C1 promoter sequence and a C1 terminator sequences operably linked to a nucleic acid sequence encoding an enzyme, such as a flippase, GNT1 or GNT2.

A particular expression construct may be assembled by a variety of different methods, including conventional molecular biology methods such as polymerase chain reaction (PCR), restriction endonuclease digestion, in vitro and in vivo assembly methods, as well as gene synthesis methods, or a combination thereof. Exemplary expression constructs and methods for their construction are provided in the Examples section below.

Deletion of alg3

Gene deletion techniques enable the partial or complete removal of a gene, thereby eliminating its expression. In such methods, deletion of the gene may be accomplished by homologous recombination using a plasmid that has been constructed to contiguously contain the 5′ and 3′ regions flanking the gene.

Gene deletion may also be performed by inserting into the gene a disruptive nucleic acid construct, also termed herein a deletion construct. A disruptive construct may be simply a selectable marker gene accompanied by 5′ and 3′ regions homologous to the gene. The selectable marker enables identification of transformants containing the disrupted gene. Alternatively or additionally, the disruptive nucleic acid construct may comprise one or more polynucleotides encoding heterologous proteins to be expressed in the host cell.

Exemplary deletion constructs for alg3 and procedures for carrying out the deletion are described for example, in WO 2021/094935. The deletion(s) may be confirmed using PCR with appropriate primers flanking the disruptive construct(s).

In some embodiments, the Th. heterothallica of the present invention is genetically modified to express a heterologous flippase. In some particular embodiments, the heterologous flippase is a yeast flippase. In additional particular embodiments, the yeast flippase is the S. cerevisiae mutant flippase FLC2p, which is a C-terminally truncated version of the S. cerevisiae ER-localized flippase FLC2.

In additional embodiments, the Th. heterothallica of the present invention is genetically modified to over-express the endogenous Th. heterothallica RFT1 flippase. In some particular embodiments. Th. heterothallica C1 is genetically modified to over-express the endogenous C1 RFT1 flippase. Over-expression of RFT1 in Th. heterothallica according to the present invention may be performed by the introduction of an exogenous polynucleotide encoding RFT1, comprising the nucleic acid sequence encoding RFT1 operably linked to regulatory sequences operable in Th. heterothallica. An exemplary nucleotide sequence of rft 1 is set forth in SEQ ID NO: 7. An exemplary amino acid sequence of rft1 is set forth in SEQ ID NO: 8.

In some exemplary embodiments, the Th. heterothallica is genetically modified to delete or disrupt alg3, to express Mannosidase 1 (alpha-1,2-Mannosidase) and ER-targeted C1 Glucosidase 2 alpha-subunit and to over-express the endogenous Th. heterothallica RFT1 flippase, and further genetically modified to express an animal-derived GNT1 comprising a heterologous Golgi localization signal and an animal-derived GNT2, for example, to express human GNT1 comprising a Golgi localization signal from the yeast protein KRE2 and human GNT2.

In some exemplary embodiments, the Th. heterothallica is genetically modified to delete or disrupt alg3, to express Mannosidase 1 (alpha-1,2-Mannosidase) and Glucosidase 2 alpha-subunit and to express the yeast FLC2p flippase, and further genetically modified to express an animal-derived GNT1 comprising a heterologous Golgi localization signal and an animal-derived GNT2, for example to express human GNT1 comprising a Golgi localization signal from the Th. heterothallica protein KRE2 and rat GNT2.

In additional exemplary embodiments, the Th. heterothallica is genetically modified by: deletion or disruption of alg3; expression of Mannosidase 1 (alpha-1,2-Mannosidase) and ER-targeted C1 Glucosidase 2 alpha-subunit; over-expression of the endogenous Th. heterothallica RFT1 flippase; expression of human GNT1 comprising Th. heterothallica KRE2a Golgi-localization signal and rat GNT2; and expression of Leishmania major STT3. In some embodiments, such a Th. heterothallica is further genetically modified by expression of human galactosyltransferase or Xenopus tropicalis galactosyltransferase.

In additional exemplary embodiments, the Th. heterothallica is genetically modified by: deletion or disruption of alg3; expression of Mannosidase 1 (alpha-1,2-Mannosidase) and ER-targeted C1 Glucosidase 2 alpha-subunit; over-expression of the endogenous Th. heterothallica RFT1 flippase; expression of bovine GNT1 comprising Th. heterothallica KRE2 Golgi-localization signal and rat GNT2; and expression of Leishmania major STT3.

### Mannosidase 1 (Alpha-1,2-Mannosidase) and Glucosidase 2 Alpha-Subunit

According to some embodiments, expression constructs of ER-targeted Mannosidase 1 (alpha-1,2-Mannosidase) and ER-targeted C1 Glucosidase 2 alpha-subunit are integrated to the alp3 protease locus of Thermothelomyces heterothallica.

An exemplary nucleotide sequence of ER-targeted Trichoderma reesei Mannosidase 1 is set forth in SEQ ID NO: 1. An exemplary amino acid sequence of ER-targeted Trichoderma reesei Mannosidase 1 is set forth in SEQ ID NO: 2.

An exemplary nucleotide sequence of ER-targeted C1 Glucosidase 2 alpha-subunit (gls2a-HDEL) is set forth in SEQ ID NO: 3. An exemplary amino acid sequence of ER-targeted C1 Glucosidase 2 alpha-subunit is set forth in SEQ ID NO: 4.

### GNT1 and GNT2

In some embodiments, the Th. heterothallica of the present invention is genetically modified to express heterologous GNT1 and GNT2. In some embodiments, the heterologous GNT1 and GNT2 are animal-derived. As used herein, “animal-derived” encompasses mammalian origin including for example companion animals such as dogs and cats and additional mammals such as horses. As exemplified herein below, animal-derived includes for example a rat origin. The term “animal-derived” further encompasses human-derived, as further exemplified hereinbelow.

The heterologous GNT1 and GNT2 may be expressed in Th. heterothallica according to the present invention by the introduction of one or more exogenous polynucleotide encoding the GNT1 and GNT2, comprising nucleic acid sequences encoding the GNT1 and GNT2 operably linked to regulatory sequences operable in Th. heterothallica. In some embodiments, the nucleic acid sequences encoding the GNT1 and GNT2 are included in a single polynucleotide that is introduced into the Th. heterothallica. In other embodiments. the nucleic acid sequences encoding the GNT1 and GNT2 are included in two different polynucleotides that are introduced into the Th. heterothallica.

In some embodiments, the GNT1 is expressed in the Th. heterothallica with its own naturally-occurring Golgi-localization signal. In other embodiments, the GNT1 is expressed in the Th. heterothallica with a heterologous Golgi-localization signal.

In some embodiments, the heterologous Golgi-localization signal is a yeast Golgi-localization signal. In some particular embodiments, the heterologous Golgi-localization signal is from the yeast protein KRE2 alpha-1,2-mannosyltransferase. In some exemplary embodiments, the heterologous Golgi-localization signal is from the KRE2 of S. cerevisiae.

In other embodiments, the heterologous Golgi-localization signal is from a filamentous fungus. In some embodiments, the heterologous Golgi-localization signal is from Th. heterothallica. In some particular embodiments, the heterologous Golgi-localization signal is from the C1 homolog of the yeast protein KRE2.

In some embodiments, the GNT1 is human GNT1. In some embodiments, the human GNT1 that is introduced into the Th. heterothallica comprises a heterologous Golgi-localization signal. In some embodiments, the GNT1 is human GNT1 comprising a yeast Golgi-localization signal. In some particular embodiments, the GNT1 is human GNT1comprising the Golgi-localization signal from the protein KRE2 of S. cerevisiae. An exemplary nucleotide sequence of KRE2 signal-GNT1 is set forth in SEQ ID NO: 5. An exemplary amino acid sequence of KRE2 signal-GNT1 is set forth in SEQ ID NO: 6.

In some embodiments, the GNT2 is rat GNT2. GNT2 is typically expressed with its own naturally-occurring Golgi localization signal. The amino acid sequence of rat GNT2 is set forth in SEQ ID NO: 16. An exemplary nucleic acid sequence of a polynucleotide for use according to the present invention encoding rat GNT2 is set forth in SEQ ID NO: 15.

In other embodiments, the GNT2 is human GNT2. GNT2 is typically expressed with its own naturally-occurring Golgi localization signal.

Exemplary but not limiting combinations of GNT1 and GNT2 according to the present invention include:
human GNT1 with yeast KRE2 Golgi-localization signal and human GNT2;
human GNT1 with yeast KRE2 Golgi-localization signal and rat GNT2;
human GNT1 with C1 KRE2a Golgi-localization signal and human GNT2;
human GNT1 with C1 KRE2a Golgi-localization signal and rat GNT2;
bovine GNT1 with C1 KRE2a Golgi-localization signal and rat GNT2.

Each combination represents a separate embodiment of the present invention.

### Galactosyltransferase

In some embodiments, the Th. heterothallica of the present invention is genetically modified to express a heterologous galactosyltransferase. In some embodiments, the heterologous galactosyltransferase is animal-derived.

A galactosyltransferase may be expressed in Th. heterothallica according to the present invention by the introduction of an exogenous polynucleotide encoding the galactosyltransferase, comprising the nucleic acid sequence e encoding the galactosyltransferase operably linked to regulatory sequences operable in Th. heterothallica.

In some embodiments, the galactosyltransferase is expressed in the Th. heterothallica with its own naturally-occurring Golgi-localization signal. In other embodiments, the galactosyltransferase is expressed in the Th. heterothallica with a heterologous Golgi-localization signal.

In some embodiments, the galactosyltransferase is a human galactosyltransferase (huGalT1). In some embodiments, the human galactosyltransferase that is introduced into the Th. heterothallica comprises a heterologous Golgi-localization signal. In some particular embodiments, the human galactosyltransferase comprises the S. cerevisiae KRE2 Golgi-localization signal. The amino acid sequence of human galactosyltransferase with the Golgi-localization signal from the protein KRE2 of S. cerevisiae is set forth in SEQ ID NO: 14. An exemplary nucleic acid sequence of a polynucleotide for use according to the present invention encoding human galactosyltransferase with the Golgi-localization signal from the protein KRE2 of S. cerevisiae is set forth in SEQ ID NO: 13.

According to other embodiments, the galactosyltransferase is from Xenopus tropicalis (XtGalT1). In some embodiments, the galactosyltransferase from Xenopus tropicalis that is introduced into the Th. heterothallica comprises a heterologous Golgi-localization signal. In some particular embodiments, the galactosyltransferase from Xenopus tropicalis comprises the Golgi-localization signal from the protein KRE2 of S. cerevisiae.

### STT3 Oligosaccharyltransferase

In some embodiments, the Th. heterothallica of the present invention is genetically modified to express a heterologous STT3 subunit of oligosaccharyltransferase. In some particular embodiments, the heterologous STT3 is Leishmania major STT3. The amino acid sequence of Leishmania major STT3 is set forth in SEQ ID NO: 12. Leishmania major STT3 may be expressed in Th. heterothallica according to the present invention by the introduction of an exogenous polynucleotide encoding Leishmania major STT3, comprising the nucleic acid sequence encoding Leishmania major STT3 operably linked to regulatory sequences operable in Th. heterothallica. An exemplary nucleic acid sequence encoding Leishmania major STT3 for use according to the present invention is set forth in SEQ ID NO: 11.

Genetically-Engineered Th. heterothallica

Th. heterothallica cells genetically engineered to produce glycoproteins with N-glycans of mammalian proteins (particularly human and companion animal proteins) according to the present invention are generated by modifying, such as deleting, the endogenous gene of Th. heterothallica alg3, such that the genes fail to produce functional proteins, and expressing exogenous polynucleotides encoding various enzymes.

It is to be understood that the genetic modification of Th. heterothallica as disclosed herein does not necessarily requires that each and every cell of the genetically-modified Th. heterothallica be modified, as long as the desired outcome disclosed herein of production of glycoproteins with N-glycans of mammalian proteins (particularly human and companion animal proteins) is obtained.

In some embodiments, the Th. heterothallica is further genetically modified to express a heterologous mammalian glycoprotein. In some embodiments, the heterologous mammalian glycoprotein is an antibody or an antigen-binding fragment thereof.

In some exemplary embodiments, the Th. heterothallica is genetically modified to express Nivolumab or an antigen-binding fragment thereof. In additional exemplary embodiments, the Th. heterothallica is genetically modified to express Nivolumab light chain with Th. heterothallica CBH1 signal sequence and Nivolumab heavy chain with Th. heterothallica CBH1 signal sequence.

In some embodiments, the present invention provides a Th. heterothallica cell genetically modified as disclosed herein.

The expression of an exogenous polynucleotide is carried out by introducing into Th. heterothallica cells, particularly into the nucleus of Th. heterothallica cells, an expression construct comprising a nucleic acid encoding a protein to be expressed in C1. In particular, the genetic modification according to the present invention means incorporation of the expression construct to the host genome.

Introduction of an expression construct into Th. heterothallica cells, i.e., transformation of Th. heterothallica, can be performed by methods for transforming filamentous fungi.

To facilitate easy selection of transformed cells, a selection marker may be transformed into the Th. heterothallica cells. A “selection marker” indicates a polynucleotide encoding a gene product conferring a specific type of phenotype that is not present in non-transformed cells, such as an antibiotic resistance (resistance markers), ability to utilize a certain resource (utilization/auxotrophic markers) or expression of a reporter protein that can be detected, e.g. by spectral measurements. Auxotrophic markers are typically preferred as a means of selection in the food or pharmaceutical industry. The selection marker can be on a separate polynucleotide co-transformed with the expression construct, or on the same polynucleotide of the expression construct. Following transformation, positive transformants are selected by culturing the C1 cells on e.g., selective media according to the chosen selection marker. In some cases, a split marker system is used, where the selection marker is split into two plasmids and a functional selection marker is formed only when the two plasmids are co-transformed and joined together via homologous recombination.

When the synthetic expression system is used, an expression cassette coding for a suitable synthetic transcription factor (sTF) is introduced into the host cell.

The transformed DNA may integrate into Th. heterothallica chromosomes through homologous recombination or non-homologous end joining. To facilitate targeted integration into a specific locus in the genome, sequences corresponding to the target locus are incorporated into the same polynucleotide with the expression construct.

Selected clones are then grown and examined for the production of protein with the desired N-glycoforms. The genetically-modified Th. heterothallica is cultured under suitable conditions. According to certain embodiments, the fungus is grown at a temperature in the range of from about 20° C. to about 45° C. and at a medium pH of from about 4.0 to about 8.0. Particular media types may be selected according to regulatory requirements of the end product. The produced glycoproteins may be isolated and analyzed.

Expression of GNT1, GNT2 and optionally additional enzymes such as a galactosyltransferase in the Th. heterothallica may be determined by structural analysis of N-glycans produced by the C1.

A Th. heterothallica genetically modified according to the present invention produces G0 (Man₃GlcNAc₂) as a final N-glycan structure or an intermediate N-glycan structure.

In some embodiments, a Th. heterothallica genetically modified by deletion or disruption of alg3, expression of ER-targeted Trichoderma reesei Mannosidase 1 (alpha-1,2-Mannosidase) and ER-targeted C1 Glucosidase 2 alpha-subunit, optionally expression of a heterologous flippase or over-expression of an endogenous flippase, expression of heterologous GNT1 and GNT2 and optionally expression of a heterologous STT3 oligosaccharyltransferase produces secreted glycoproteins wherein G0 constitutes at least 80% of the N-glycans on the secreted glycoproteins, preferably at least 85%, at least 90%, at least 91%, at least 92%, at least 93%, at least 94% or even at least 95% of the N-glycans on the secreted glycoproteins. Each value represents a separate embodiment of the present invention.

In some embodiments, a Th. heterothallica which is further genetically modified to express a heterologous galactosyltransferase produces secreted glycoproteins wherein G1 and G2 (total of both G1 and G2) constitute at least 75% of the N-glycans on the secreted glycoproteins, preferably at least 80%, at least 85%, at least 90%, at least 91%, at least 92%, at least 93%, at least 94% or even at least 95% of the N-glycans on the secreted glycoproteins. Each value represents a separate embodiment of the present invention.

In some embodiments, the genetic modification of the Th. heterothallica does not include expression of a heterologous oligosaccharyltransferase (OST).

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1—Generating a Nivolumab Producing Strain in Three Steps With Humanized G1/2 Glycans

The first step in generating a Thermothelomyces heterothallica C1 strain producing human type galactosylated glycans was deletion of the alg3 gene from a strain carrying deletions of 8 protease genes, and expressing Nivolumab (described in WO 2021/094935). After the alg3 deletion, the next C1 glycoengineering step was to integrate ER-targeted Trichoderma reesei Mannosidase 1 (alpha-1,2-Mannosidase) and ER-targeted C1 Glucosidase 2 alpha-subunit to alp3 protease locus in order to trim down the glycan precursor to DolP-GlcNAc₂-Man₃ that is suitable for synthesis of human-type glycans. Thereafter expression cassettes containing human or bovine GNT1 (GlcNAc transferase I), rat GNT2 (GlcNAc transferase II), C1 flippase RFT1, oligosaccharyl transferase STT3 from Leishmania major and human GalT1 (Galactosyltransferase I) were integrated to the alp6 protease locus. Expression of these genes results in the addition of GlcNAc residues on both branches of the GlcNAc₂Man₃ glycan resulting in G0 (GlcNAc2Man3GlcNAc2) glycan, followed by addition of galactose residue to one or both branches of G0 glycan resulting in production of G1 (GlcNAc2Man3GlcNAc2Gal) and G2 (GlcNAc2Man3MlcNAc2Gal2) glycans.

In the first transformation step, the DNA constructs designed to integrate to alp3 locus (JGI M. thermophila genome database ID2306020) and to simultaneously express T. reesi (Tr) mns1-HDEL (JGI T. reesei genome database ID45717) and C1 gls2a-HDEL (JGI M. thermophila genome database ID2125259) were constructed in two parts into two separate plasmids. HDEL is the four amino acid C-terminal ER localization signal. The first plasmid contained the alp3 5′ flanking region fragment for integration, an expression cassette for Tr mns1-HDEL where the gene is between C1 bgl8 promoter and terminator (JGI M. thermophila genome database ID115968), a synthetic transcription factor (sTF, for the synthetic promoter in the 3′ plasmid), a direct repeat to C1 cbh1 terminator (JGI M. thermophila genome database ID109566) and the first ⅔ of the amdS marker gene (encoding acetamidase of Aspergillus nidulans). The second plasmid contained the last 2/3of the amdS marker, a direct repeat fragment targeted to the end of the sTF cassette (for amdS marker removal by recombination), an expression cassette for C1 gls2a-HDEL between the synthetic AnSES promoter (Rantasalo A et al. 2018, A universal gene expression system for fungi. Nucl. Acids Research 46 (18): e111) and chil terminator (JGI M. thermophila genome database ID50608) and the alp3 3′ flanking region fragment for integration. The amdS marker fragments in these two plasmids overlap with each other, and this region undergoes homologous recombination in C1 between the plasmids at the same time as the 5′ and 3′ flanking region fragments recombine with genomic DNA on both sides of the alp3 gene. The recombination between the selection marker fragments makes the marker gene functional and enables the transformants to grow under selection.

A construct containing the alp3 5′ flanking region fragment, the expression cassette for Tr mns1-HDEL, sTF and the first ⅔ of the amdS marker is set forth in SEQ ID NO: 17 (pMYT1288). The 5′ flank sequence corresponds to positions 9-1196 of SEQ ID NO: 17. The bgl8 promoter sequence corresponds to positions 1202-2593 of SEQ ID NO: 17. A nucleic acid sequence encoding genomic Trichoderma reesei Mannosidase 1 with artificial HDEL ER-retention signal corresponds to positions 2594-4420 of SEQ ID NO: 17. The nucleic acid sequence encoding T. reesei MNS1 with HDEL-signal is also set forth as SEQ ID NO: 1 (Tr mns1-HDEL nt) and the amino acid sequence as SEQ ID NO: 2 (Tr MNS1-HDEL aa). The bgl8 terminator sequence corresponds to positions 4421-4887 of SEQ ID NO: 17. The nucleic acid sequence for the synthetic transcription factor cassette corresponds to positions 4901-6550 of SEQ ID NO: 17. The first ⅔ of the amdS marker gene corresponds to positions 7060-9126 of SEQ ID NO: 17. The fragments were produced by PCR, separated in agarose gel, purified and cloned by Gibson Assembly (NEBuilder® HiFi DNA Assembly Cloning Kit, New England Biolabs) method into the backbone vector pRS426 to get the 5′ arm expression vector pMYT1288 that was verified by sequencing.

A construct containing the last ⅔ of the amdS marker, an expression cassette for C1 gls2a-HDEL and the alp3 3′ flanking region fragment for integration is set forth in SEQ ID NO: 18 (pMYT0721). The ⅔ of the amdS marker gene corresponds to positions 17-1738 of SEQ ID NO: 18. The synthetic AnSES promoter sequence corresponds to positions 2255-2747 of SEQ ID NO: 18. The sequence encoding C1 Glucosidase 2 alpha with artificial HDEL ER-retention signal corresponds to positions 2748-5760 of SEQ ID NO: 18. The nucleic acid sequence encoding C1 GLS2alpha with HDEL-signal is also set forth as SEQ ID NO: 3 (C1 gls2a-HDEL nt) and the amino acid sequence as SEQ ID NO: 4 (C1 GLS2A-HDEL aa). The chi1 terminator sequence corresponds to positions 5761-6406 of SEQ ID No: 18. The alp3 3′ flank sequence corresponds to positions 6415-7548 of SEQ ID NO: 18. Fragments were produced by PCR, separated in agarose gel, purified and cloned by Gibson Assembly (NEBuilder® HiFi DNA Assembly Cloning Kit, New England Biolabs) method into the backbone vector pRS426 to get the 3′ arm expression vector pMYT0721 that was verified by sequencing.

For the second transformation step, the DNA constructs designed to integrate to alp6 locus (JGI M. thermophila genome database ID94536) and simultaneously express GNT1 and GNT2, as well as RFT1, STT3 and GalT1, were constructed in two parts into two separate plasmids. The first plasmid contained the alp6 5′ flanking region fragment for integration, an expression cassette for GNT1 where either human or bovine GNT1 gene fused to C1 KRE2 Golgi localization signal (JGI M. thermophila genome database ID2300989) between C1 bgl8 promoter and bgl8 terminator, the C1 flippase RFT1 (JGI M. thermophila genome database ID2307799) between promoter and terminator of the ubiquitin-like protein gene (JGI M. thermophila genome database ID2315548), and the first ⅔ of the C1 pyr4 marker gene (JGI M. thermophila genome database ID2311494). The second plasmid contained the last ⅔ of the C1 pyr4 marker gene, a direct repeat fragment targeted to the beginning of pyr4 cassette (for pyr4 marker removal by recombination) reversed expression cassettes for STT3 from Leishmania major between the promoter and terminator of a C1 hypothetical protein (JGI M. thermophila genome database ID2315630), for human GalT1 fused to Saccharomyces cerevisiae KRE2 Golgi localization signal between the promoter of the ubiquitin-like protein gene and bgl8 terminator, the GNT2 gene from rat between a promoter of translation elongation factor 1A (JGI M. thermophila genome database ID2298136) and terminator of a C1 hypothetical protein (JGI M. thermophila genome database ID114107), and finally the alp6 3′ flanking region fragment for integration. The C1 pyr4 marker fragments in these two plasmids overlap with each other, and this region undergoes homologous recombination in C1 between the plasmids at the same time as the 5′ and 3′ flanking region fragments recombine with genomic DNA on both sides of the alp6 gene. The recombination between the selection marker fragments makes the marker gene functional and enables the transformants to grow under selection.

The first 5′ construct containing the alp6 5′ flanking region fragment, the expression cassette for human GNT1, C1 RFT1 and the first ⅔ of the C1 pyr4 marker gene are set forth as SEQ ID NO: 19 (pMYT1451). The 5′ flank sequence corresponds to positions 8-1156 of SEQ ID NO: 19. The bgl8 promoter sequence corresponds to positions 1165-2556 of SEQ ID NO: 19. A nucleic acid sequence encoding human GNT1 fused to C1 KRE2 Golgi-localization signal corresponds to positions 2557-4042 of SEQ ID NO: 19, where positions 2557-2821 encode the C1 KRE2 localization signal and positions 2822-4042 encode the human GNT1. The nucleic acid sequence encoding human GNT1 fused to C1 KRE2 Golgi-localization signal is also set forth as SEQ ID NO: 5 (C1 kre2-huGNT1 nt). The nucleic acid sequence encoding human GNT1 was codon-optimized for M. thermophila C1 and synthetized by GenScript (USA). In the synthesized sequence the region of amino acids 1-100 of human GNT1 was replaced by the C1 KRE2 Golgi localization signal (amino acids 1-70). The full amino acid sequence of C1 KRE2-GNT1 is set forth as SEQ ID NO: 6 (C1 KRE2-huGNT1 aa). The bgl8 terminator sequence corresponds to positions 4046-4512 of SEQ ID NO: 19. The ubiquitin-like protein promoter sequence corresponds to positions 4513-5532 of SEQ ID NO: 19. The C1 flippase rft1 gene corresponds to positions 5533-7449 of SEQ ID NO: 19. The nucleic acid sequence encoding C1 RFT1 is also set forth as SEQ ID NO: 7 (C1 rft1 nt) and the amino acid sequence as SEQ ID NO: 8 (C1 RFT1 aa). The ubiquitin-like protein terminator sequence corresponds to positions 7458-7953 of SEQ ID NO: 19. The first ⅔ of the C1 pyr4 marker gene corresponds to positions 7969-9748 of SEQ ID No: 19.

The second 5′ construct containing the alp6 5′ flanking region fragment, the expression cassette for bovine GNT1, C1 RFT1 and the first ⅔ of the C1 pyr4 marker gene are set forth as SEQ ID NO: 20 (pMYT1452). The 5′ flank sequence corresponds to positions 8-1156 of SEQ ID NO: 20. The bg18 promoter sequence corresponds to positions 1165-2556 of SEQ ID NO: 20. A nucleic acid sequence encoding bovine GNT1 fused to C1 KRE2 Golgi-localization signal corresponds to positions 2557-4051 of SEQ ID NO: 20, where positions 2557-2821 encode the C1 KRE2 localization signal and positions 2822-4051 encode the bovine GNT1. The nucleic acid sequence encoding bovine GNT1 fused to C1 KRE2 Golgi-localization signal is also set forth as SEQ ID NO: 9 (C1 kre2-boGNT1 nt).

The nucleic acid sequence encoding bovine GNT1 was codon-optimized for M. thermophila C1 and synthetized by GenScript (USA). In the synthesized sequence the region of amino acids 1-38 of bovine GNT1 were removed and replaced by the C1 KRE2 Golgi localization signal (amino acids 1-70) upon cloning of the expression plasmid. The full amino acid sequence of C1 KRE2-GNT1 is set forth as SEQ ID NO: 10 (C1 KRE2-boGNT1 aa). The bgl8 terminator sequence corresponds to positions 4052-4518 of SEQ ID NO: 20. The ubiquitin-like protein promoter sequence corresponds to positions 4524-5543 of SEQ ID NO: 20. The C1 flippase RFT1 gene described for pMYT1451 corresponds to positions 5544-7460 of SEQ ID NO: 20. The ubiquitin-like protein terminator sequence corresponds to positions 7469-7964 of SEQ ID NO: 20. The first ⅔ of the C1 pyr4 marker gene corresponds to positions 7980-9759 of SEQ ID NO: 20. Fragments for both 5′plasmids were produced by PCR, separated in agarose gel, purified and cloned by Gibson Assembly (NEBuilder® HiFi DNA Assembly Cloning Kit, New England Biolabs) method into the backbone vector pRS426 to get the 5′ arm expression vectors pMYT1451 and pMYT1452 verified by sequencing.

A construct containing the last ⅔ of the C1 pyr4 marker gene, a reversed expression cassette for the STT3 from Leishmania major, a reversed expression cassette for the human GalT1, a reversed expression cassette for the rat GNT2 gene and the alp6 3′ flanking region fragment for integration is set forth in SEQ ID NO: 21 (pMYT1453). The ⅔ of the C1 pyr4 marker gene corresponds to positions 17-1273 of SEQ ID NO: 21. The hypothetical protein (ID2315630) promoter sequence corresponds to positions 5965-4957 of SEQ ID NO: 21. The sequence encoding STT3 from Leishmania major, codon-optimized for M. thermophila C1 and synthetized by GenScript (USA), corresponds to positions 4956-2383 of SEQ ID NO: 21. The nucleic acid sequence encoding Leishmania major STT3 gene is also set forth as SEQ ID NO: 11 (LmSTT3 nt). The full amino acid sequence of L. major STT3 is set forth as SEQ ID NO: 12 (LmSTT3 aa). The hypothetical protein (ID2315630) terminator sequence corresponds to positions 2374-1795 of SEQ ID NO: 21. The sequence encoding human GalT1 fused to Sc KRE2 Golgi-localization signal corresponds to positions 7704-6439 of SEQ ID NO: 21, where positions 7704-7405 encode the Sc KRE2 localization signal and positions 7404-6439 encode the human GalT1. The ubiquitin-like protein promoter sequence corresponds to positions 8724-7705 of SEQ ID NO: 21. The nucleic acid sequence encoding human GalT1 fused to Sc KRE2 Golgi-localization signal is also set forth as SEQ ID NO: 13 (Sckre2-huGalT1 nt). The nucleic acid sequence encoding human GalT1 was codon-optimized for M. thermophila C1 and synthetized by GenScript (USA). In the synthesized sequence the region of amino acids 1-77 of human GalT1 were removed and replaced by the Sc KRE2 Golgi-localization signal (amino acids 1-100) upon cloning of the expression plasmid. The full amino acid sequence of Sc KRE2-GalT1 is set forth as SEQ ID NO: 14 (ScKRE2-huGalT1 aa). The bgl8 terminator sequence corresponds to positions 6438-5972 of SEQ ID NO: 21. The translation elongation factor 1A promoter sequence corresponds to positions 11618-10562 of SEQ ID NO: 21. The sequence encoding rat GNT2, codon-optimized for M. thermophila C1 and synthetized by GenScript (USA), corresponds to positions 10561-9233 of SEQ ID: 21. The nucleic acid sequence encoding rat GNT2 is also set forth as SEQ ID NO: 15 (rat GNT2 nt). The full amino acid sequence of rat GNT2 is set forth as SEQ ID NO: 16 (rat GNT2 aa). The hypothetical protein (ID114107) terminator sequence corresponds to positions 9232-8729 of SEQ ID NO: 21. The alp6 3′ flank sequence corresponds to positions 11626-12681 of SEQ ID: 21. Fragments were produced by PCR, separated in agarose gel, purified and cloned by Gibson Assembly (NEBuilder® HiFi DNA Assembly Cloning Kit, New England Biolabs) method into the backbone vector pRS426 to get the 3′ arm expression vector pMYT1453 verified by sequencing.

To obtain a Nivolumab producing strain with G1/2 N-glycans, the expression plasmids described above were transformed consecutively into the pyr4-minus Nivolumab producing alg3 deletion strain M3291 (described in WO 2021/094935). In each transformation a pair of one 5′arm expression vector and one 3′arm expression vector (excised from the expression plasmid backbones with MssI) was used. The pairs were:
First round: SEQ ID NO: 17 (pMYT1288) (alp3 5′flanking region−Tr Mns1-HDEL cassette−STF cassette−⅔ amdS)+SEQ ID NO: 18 (pMYT0721) (⅔ amdS−C1 gls2a-HDEL cassette−alp3 3′ flanking fragment)
Second round: SEQ ID NO: 19 (pMYT1451) or SEQ ID NO: 20 (pMYT1452) (alp6 5′flanking region−human or bovine GNT1 cassette−C1 RFT1 cassette−⅔ pyr4)+SEQ ID NO: 21 (pMYT1453) (⅔ pyr4−LmSTT3 cassette−human GalT1 cassette−rat GNT2 cassette−alp6 3′ flanking fragment)

The C1 transformation and transformant selection were done essentially as described in WO 2021/094935. For the first round of transformation selection was based on functional amdS gene, for the second round on functional pyr4 gene. The transformants were screened by PCR to find clones where alp3 deletion site (first round) or alp6 gene (second round) had been replaced by the construct.

The correct integration of the plasmid/genes to the transformants genome was verified with specific primenrs. Transformants of the first round having the correct integration of the expression constructs and further deletion of the alp3 locus were stored as M4855 and M4856. Strain M4855 was used for second round of transformation. The transformants of the second round having the correct integration of the constructs and loss of the alp6 gene were stored as M5129 and M5130 (with human GNT1), and as M5131 and M5132 (with bovine GNT1), respectively.

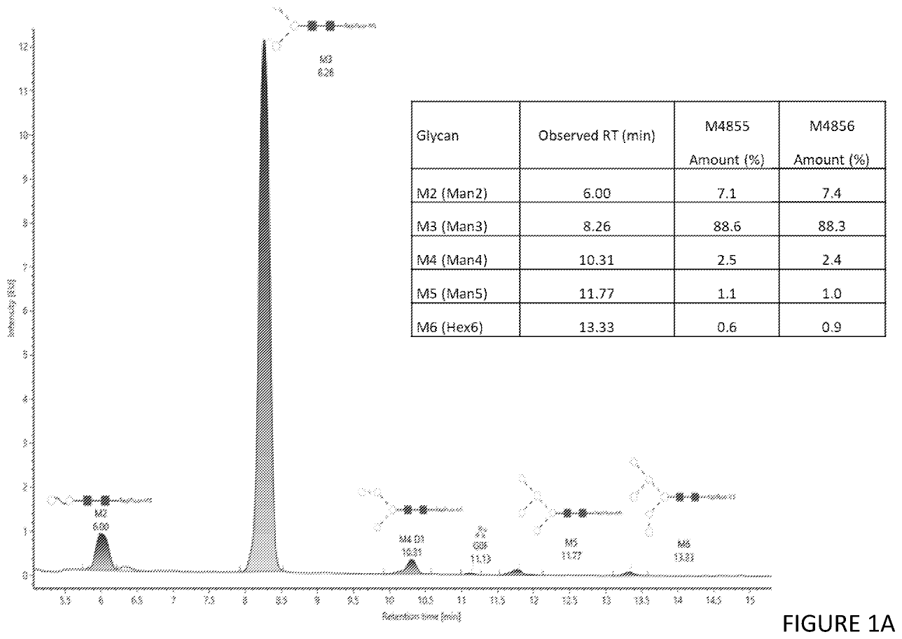

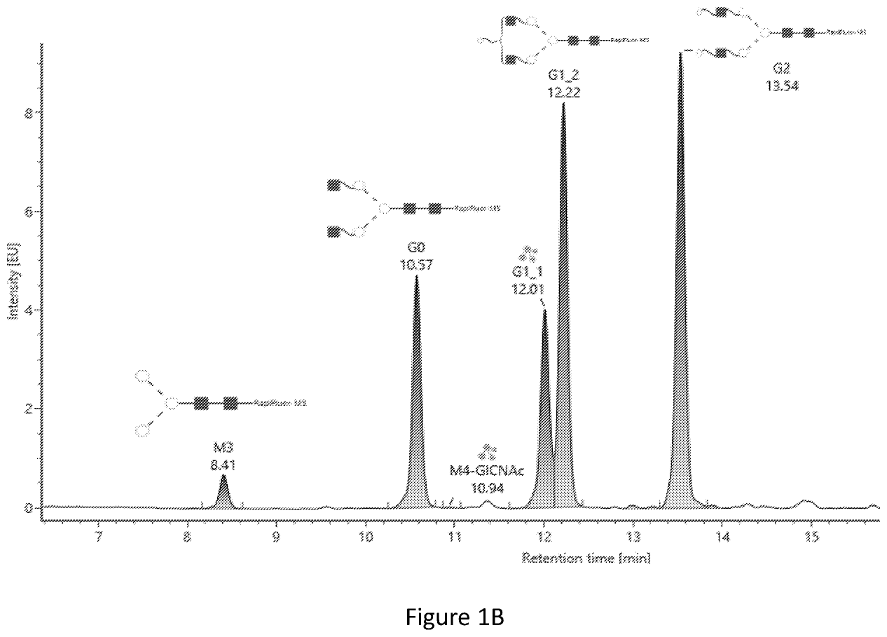

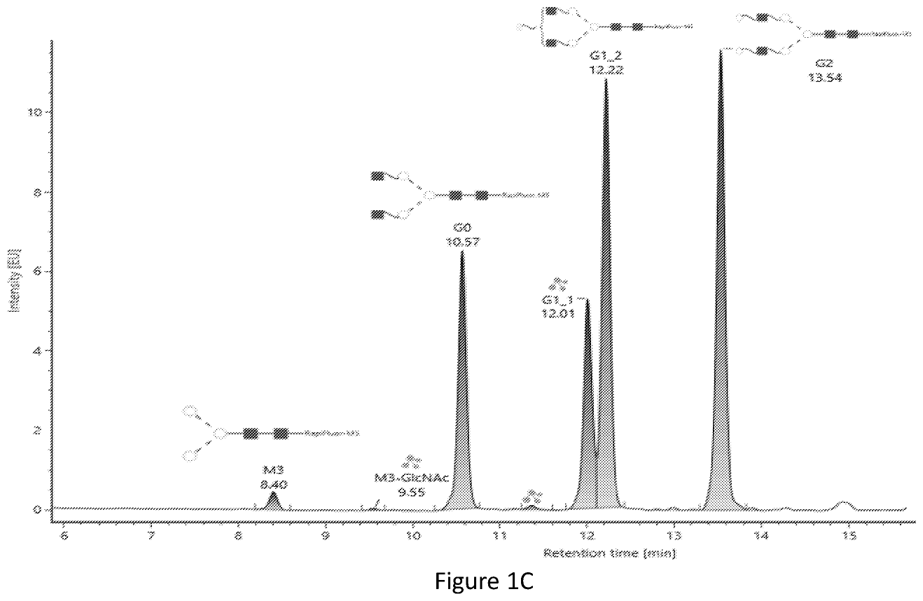

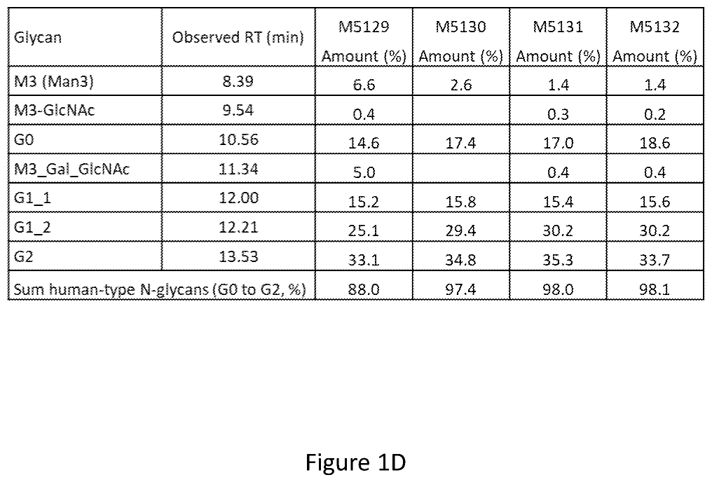

The constructed C1 strains from both transformation rounds were grown in 250 ml shake flasks in 50 ml of a liquid medium as described for the shake flask cultivation of parental strain M3291 in WO 2021/094935. The cultures were carried out at 35° C., ˜200 RPM for 4 days. Mycelia were removed by centrifuging and the supernatants from cultivations were used in Protein A affinity purification of Nivolumab using ÄKTA Start automated HPLC system (Cytiva) and HiTrap MabSelect Sure or HiTrap MabSelect PrismA prepacked 1 ml columns according to manufacturer's (Cytiva) instructions. Peak fractions from all samples were subjected to released N-glycan analysis as described in Example 1 of WO 2021/094935. The results are summarized in FIG. 1 for all strains. FIG. 1A shows that by expressing both Tr MNS1-HDEL and C1 GLS2A-HDEL the main N-glycan on Nivolumab is Man3 (M3) with nearly 90% amount while there is very little of higher mannose structures (GlcNAc2Man4, GlcNAc2Man5) and virtually no Hex6 (GlcNAc2Man5Glc) that is thought to block N-glycan modification to G0 and beyond.

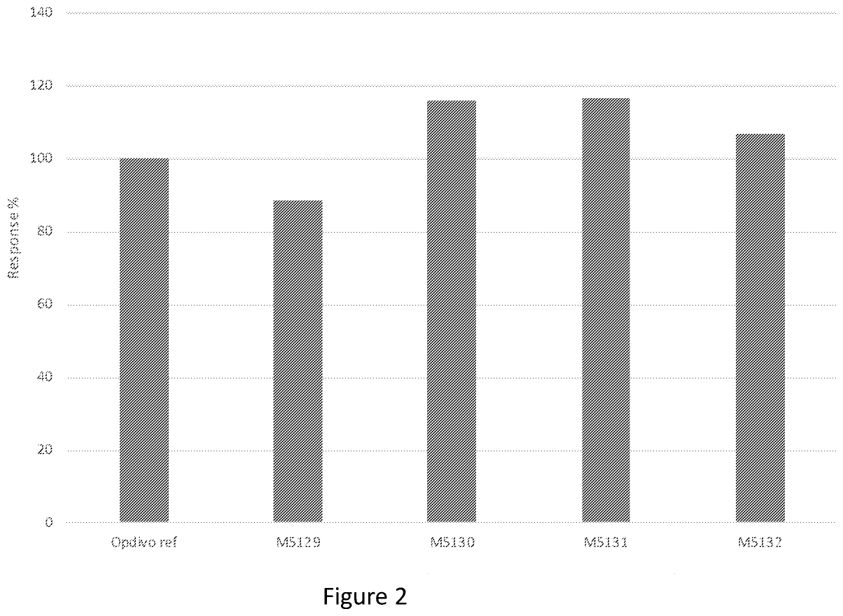

FIGS. 1B-1D show that very high G1 (GlcNAc2Man3GlcNAc2Gal) and G2 (GlcNAc2Man3GlcNAc2Gal2) N-glycan levels on target mAb are reached by using the alp6 targeted expression vectors described in this example. The conversion to human-type G1/2 N-glycans is 88-98%, i.e. nearly a full conversion to human-type glycans with this approach. In addition to the very high level of human-type N-glycans, FIG. 2 shows the amount of released N-glycans normalized between samples using a fixed amount of an internal standard added to each sample for the analysis. The percentage of released N-glycans from different Nivolumab samples was approximately on the same level as for reference Opdivo (commercial Nivolumab produced in mammalian system, Bristol-Myers Squibb) suggesting this approach to humanize glycosylation does not affect negatively to the N-glycosylation site occupancy.

Next, the C1 strains M3291, M5130 and M5132 were cultivated in ambr250 or 1 L bioreactors using a fed-batch process in a medium with yeast extract as an organic nitrogen source and glucose as a carbon source. The cultures were performed at 38° C. for seven days.

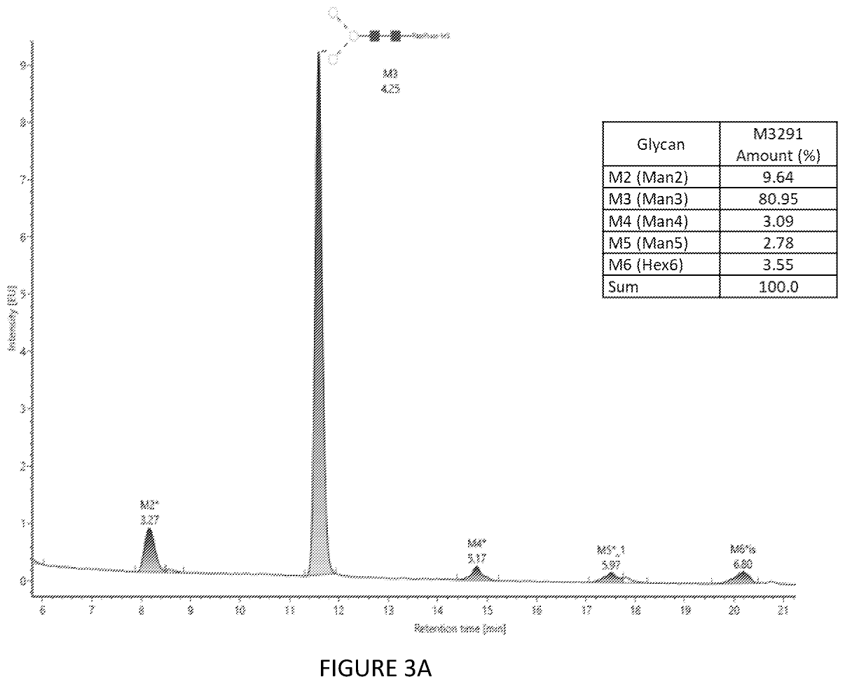

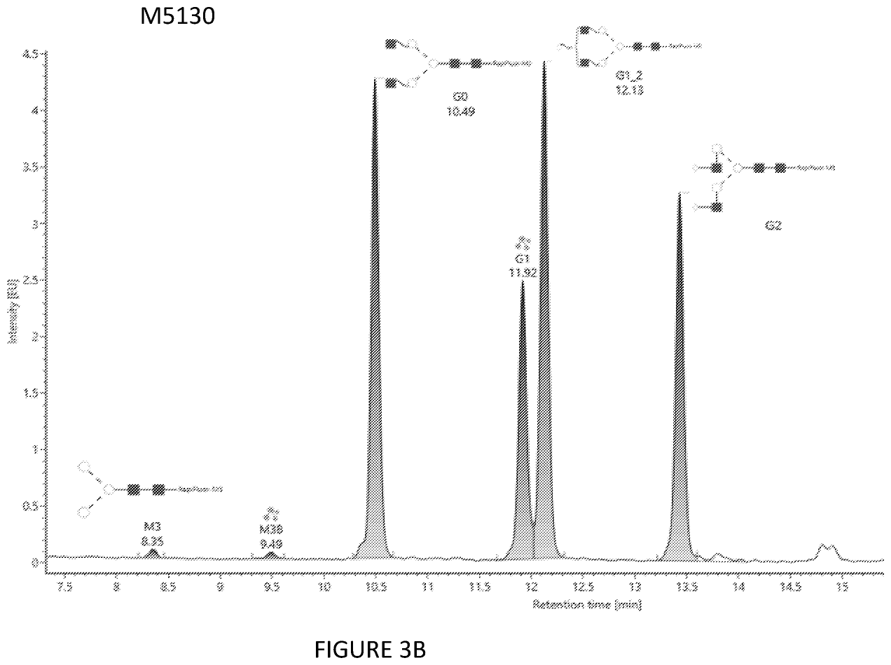

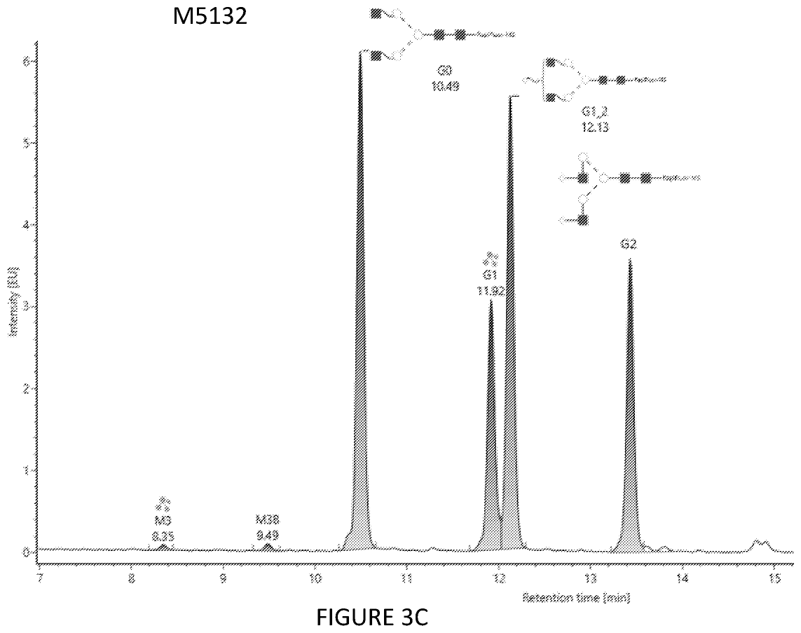

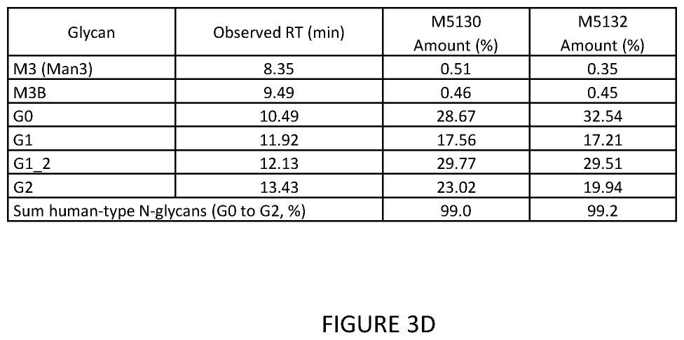

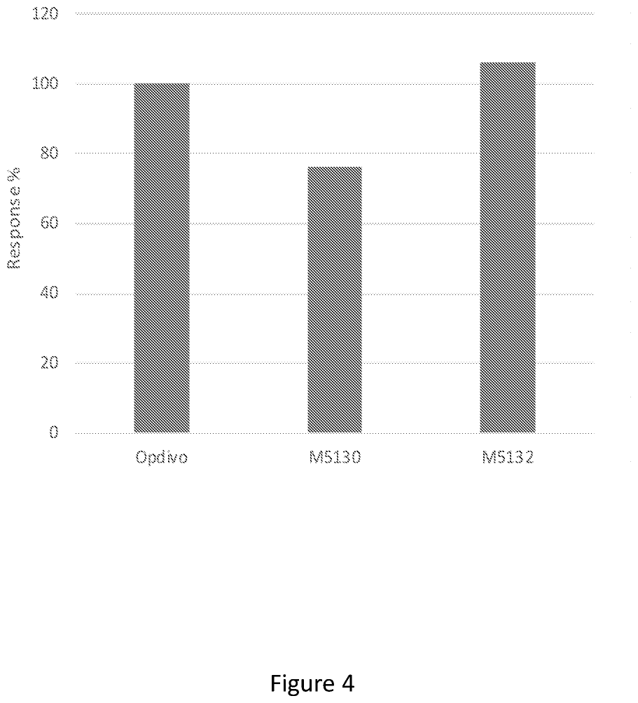

After ending the cultivation, mycelia were removed by centrifugation at 4000 g for 20 minutes, phenylmethylsulfonyl fluoride (PMSF) was added in 1-2 mM concentration to inhibit protease activity in the obtained culture supernatant and the samples were stored at −80° C. Nivolumab was purified from day seven fermentation samples using essentially the same Protein A affinity purification method as described above for the purification of the shake flask samples. The peak fractions from all samples were subjected to released N-glycan analysis. The results are summarized in FIG. 3 for all three strains. FIG. 3A shows that even though parental strain M3291 has over 80% of the wanted precursor Man3 (M3) it also has over 3.5% of Hex6 (M6) which is considered to prevent N-glycan humanization (conversion to G0 and to further modifications) and some higher Mannose structures (Man4, Man5). FIGS. 3B-3D shows that the released N-glycans on target mAb are close to fully humanized, i.e. 99% or more of the N-glycans detected belong to G0 to G2 species. FIG. 4 shows the amount of released N-glycans normalized between samples using a fixed amount of an internal standard (sialylglycan peptide) added to each sample for the analysis. Like in shake flasks, the percentage of released N-glycans from the two Nivolumab strains with humanized N-glycans seem to be at the same level as for reference Opdivo (commercial Nivolumab produced in mammalian system) further confirming the lack of negative effect on N-glycan site-occupancy.

### Example 2—Generating an Empty Strain in One Step With Humanized G1/2 Glycans

In this second approach all three glycomodification steps required to create a Thermothelomyces heterothallica C1 strain producing human type galactosylated glycans, which were described in the example 1, were combined. Shortly, deletion of the alg3 gene from a strain carrying deletions of 14 protease genes and kex2 protease gene under weaker promoter, was combined with integration of ER-targeted Trichoderma reesei Mannosidase 1 (alpha-1,2-Mannosidase), ER-targeted C1 Glucosidase 2 alpha-subunit, human GNT1 (GlcNAc transferase I), rat GNT2 (GlcNAc transferase II), oligosaccharyl transferase STT3from Leishmania major and human GalT1 (Galactosyltransferase I) to the alg3 locus. Expression of these genes results first in trimming down the glycan precursor to DolP-GlcNAc2-Man₃ that is suitable for synthesis of human-type glycans and thereafter to the addition of GlcNAc residues on both branches of the GlcNAc₂Man₃ glycan resulting in G0 (GlcNAc2Man₃GlcNAc₂) glycan, followed by addition of galactose residue to one or both branches of G0 glycan resulting in production of G1 (GlcNAc₂Man₃GlcNAc₂Gal) and G2 (GlcNAc₂Man₃MlcNAc₂Gal₂) glycans.

The DNA constructs designed to integrate to alg3 locus (JGI M. thermophila genome database ID 2310419) and to simultaneously express T. reesei (Tr) mns1-HDEL (JGI T. reesei genome database ID45717), C1 gls2a-HDEL (JGI M. thermophila genome database ID2125259), human GNT1, rat GNT2, as well as Leishmania major (Lm) STT3 and human GalT1 were constructed in two parts into two separate plasmids. HDEL stands for the four amino acid C-terminal ER localization signal.

The first plasmid contained the alg3 5′ flanking region fragment for integration, an expression cassette for GNT1 where human GNT1 gene is fused to C1 KRE2 Golgi localization signal (JGI M. thermophila genome database ID2300989) between C1 bgl8 promoter and bgl8 terminator (JGI M. thermophila genome database ID115968), for human GalT1 fused to Saccharomyces cerevisiae KRE2 Golgi localization signal between the promoter of a ubiquitin-like protein gene (JGI M. thermophila genome database ID2315548) and terminator of a C1 hypothetical protein (JGI M. thermophila genome database

ID2302731), for C1 gls2a-HDEL between the synthetic AnSES promoter (Rantasalo A et al. 2018, A universal gene expression system for fungi. Nucl. Acids Research 46 (18): c111) and chil terminator (JGI M. thermophila genome database ID50608), for a synthetic transcription factor (sTF, for the synthetic promoter of C1 gls2a-HDEL) and the first ⅔ of the pyr4 marker gene (JGI M. thermophila genome database ID2311494).

The second plasmid contained the last ⅔ of the C1 pyr4 marker gene, a direct repeat fragment targeted to the beginning of pyr4 cassette (for pyr4 marker removal by recombination), reversed expression cassettes for STT3 from Leishmania major between the promoter and terminator of a C1 hypothetical protein (JGI M. thermophila genome database ID2315630), for Tr mns-HDEL where the gene is either between C1 bg18 promoter (JGI M. thermophila genome database ID115968) or the promoter of a ubiquitin-like protein gene (JGI M. thermophila genome database ID2315548) and a ubiquitin-like protein gene terminator (JGI M. thermophila genome database ID2315548), for the GNT2 gene from rat between a promoter of translation elongation factor 1A (JGI M. thermophila genome database ID2298136) and terminator of a C1 hypothetical protein (JGI M. thermophila genome database ID114107), and finally the alg3 3′ flanking region fragment for integration. The C1 pyr4 marker fragments in these two plasmids overlap with each other, and this region undergoes homologous recombination in C1 between the plasmids at the same time as the 5′ and 3′ flanking region fragments recombine with genomic DNA on both sides of the alg3 gene. The recombination between the selection marker fragments makes the marker gene functional and enables the transformants to grow under selection.

A construct containing the alg3 5′ flanking region fragment, the expression cassettes for human GNT1, human GalT1, C1 gls2a-HDEL, sTF and the first ⅔ of the pyr4 marker is set forth in SEQ ID NO: 23 (pMYT1974). The alg3 5′ flank sequence corresponds to positions 9-1010 of SEQ ID NO: 23. The bg18 promoter sequence corresponds to positions 1018-2409 of SEQ ID NO: 23. A nucleic acid sequence encoding human GNT1 fused to C1 KRE2 Golgi-localization signal corresponds to positions 2410-3898 of SEQ ID NO: 23, where positions 2410-2674 encode the C1 KRE2 localization signal and positions 2675-3898 encode the human GNT1. The nucleic acid sequence encoding human GNT1 fused to C1 KRE2 Golgi-localization signal is also set forth as SEQ ID NO: 5 (C1 kre2-huGNT1 nt). The nucleic acid sequence encoding human GNT1 was codon-optimized for M. thermophila C1 and synthetized by GenScript (USA). In the synthesized sequence the region of amino acids 1-100 of human GNT1 was replaced by the C1 KRE2 Golgi localization signal (amino acids 1-70). The full amino acid sequence of C1 KRE2-GNT1 is set forth as SEQ ID NO: 6 (C1 KRE2-huGNT1 aa). The bgl8 terminator sequence corresponds to positions 3899-4365 of SEQ ID NO: 23. The ubiquitin-like protein promoter sequence corresponds to positions 4374-5393 of SEQ ID NO: 23. The sequence encoding human GalT1 fused to Sc KRE2 Golgi-localization signal corresponds to positions 5394-5693 of SEQ ID NO: 23, where positions 5394-5693 encode the Sc KRE2 localization signal and positions 5694-6659 encode the human GalT1. The nucleic acid sequence encoding human GalT1 fused to Sc KRE2 Golgi-localization signal is also set forth as SEQ ID NO: 13 (Sckre2-huGalT1 nt). The nucleic acid sequence encoding human GalT1 was codon-optimized for M. thermophila C1 and synthetized by GenScript (USA). In the synthesized sequence the region of amino acids 1-77 of human GalT1 were removed and replaced by the Sc KRE2 Golgi-localization signal (amino acids 1-100) upon cloning of the expression plasmid. The full amino acid sequence of Sc KRE2-GalT1 is set forth as SEQ ID NO: 14 (ScKRE2-huGalT1 aa). The terminator sequence of hypothetical protein (ID2302731) corresponds to positions 6660-7065 of SEQ ID NO: 23. The synthetic AnSES promoter sequence corresponds to positions 7073-7565 of SEQ ID NO: 23. The sequence encoding C1 Glucosidase 2 alpha with artificial HDEL ER-retention signal corresponds to positions 7566-10578 of SEQ ID NO: 23. The nucleic acid sequence encoding C1 GLS2alpha with HDEL-signal is also set forth as SEQ ID NO: 3 (C1 gls2a-HDEL nt) and the amino acid sequence as SEQ ID NO: 4 (C1 GLS2A-HDEL aa). The chil terminator sequence corresponds to positions 10579-11224 of SEQ ID NO: 23. The nucleic acid sequence for the synthetic transcription factor cassette corresponds to positions 11225-12874 of SEQ ID NO: 23. The first 2/3 of the C1 pyr4 marker gene corresponds to positions 12888-14667 of SEQ ID NO: 23. Fragments were produced by PCR, separated in agarose gel, purified and cloned stepwise using yeast recombination method into the backbone vector pRS426 to get the final 5′arm expression vector pMYT1974 that was verified by sequencing.

The two parallel constructs containing the last ⅔ of the C1 pyr4 marker gene, direct repeat for marker removal, a reversed expression cassette for the STT3 from Leishmania major, a reversed expression cassette for the Tr Mns1-HDEL, a reversed expression cassette for the rat GNT2 gene and the alg3 3′flanking region fragment for integration are set forth as SEQ ID NO: 24 (pMYT1963) and SEQ ID NO: 25 (pMYT1964). The last 2/3 of the C1 pyr4 marker gene corresponds to positions 17-1273 of SEQ ID NO: 24. The direct repeat fragment targeted to the beginning of pyr4 cassette corresponds to positions 1290-1786 of SEQ ID NO: 24. The hypothetical protein (ID2315630) promoter sequence corresponds to positions 5965-4957 of SEQ ID NO: 24. The sequence encoding STT3 from Leishmania major, codon-optimized for T. heterothallica C1 and synthetized by GenScript (USA), corresponds to positions 4956-2383 of SEQ ID NO: 24. The nucleic acid sequence encoding Leishmania major STT3 gene is also set forth as SEQ ID NO: 11 (LmSTT3 nt). The full amino acid sequence of L. major STT3 is set forth as SEQ ID NO: 12 (LmSTT3 aa). The hypothetical protein (ID2315630) terminator sequence corresponds to positions 2374-1795 of SEQ ID NO: 24. The ubiquitin-like protein promoter sequence corresponds to positions 9316-8297 of SEQ ID NO: 24. A nucleic acid sequence encoding genomic Trichoderma reesei Mannosidase 1 with artificial HDEL ER-retention signal corresponds to positions 8296-6470 of SEQ ID NO: 24. The nucleic acid sequence encoding T. reesei MNS1 with HDEL-signal is also set forth as SEQ ID NO: 1 (Tr mns1-HDEL nt) and the amino acid sequence as SEQ ID NO: 2 (Tr MNS1-HDEL aa). The ubiquitin-like protein terminator sequence corresponds to positions 6469-5974 of SEQ ID NO: 24. The translation elongation factor 1A promoter sequence corresponds to positions 12210-11154 of SEQ ID NO: 24. The sequence encoding rat GNT2, codon-optimized for T. heterothallica C1 and synthetized by GenScript (USA), corresponds to positions 11153-9825 of SEQ ID NO: 24. The nucleic acid sequence encoding rat GNT2 is also set forth as SEQ ID NO: 15 (rat GNT2 nt). The full amino acid sequence of rat GNT2 is set forth as SEQ ID NO: 16 (rat GNT2 aa). The hypothetical protein (ID114107) terminator sequence corresponds to positions 9824-9321 of SEQ ID NO: 24. The alg3 3′flank sequence corresponds to positions 12218-13217 of SEQ ID NO: 24. Fragments were produced by PCR, separated in agarose gel, purified and cloned stepwise using yeast recombination method into the backbone vector pRS426 to get the final 3′arm expression vector pMYT1963 that was verified by sequencing.

In SEQ ID NO: 25 (pMYT1964) the last ⅔ of the C1 pyr4 marker gene corresponds to positions 17-1273. The direct repeat fragment targeted to the beginning of pyr4 cassette corresponds to positions 1290-1786 of SEQ ID NO: 25. The hypothetical protein (ID2315630) promoter sequence corresponds to positions 5965-4957 of SEQ ID NO: 25. The sequence encoding STT3 from Leishmania major, codon-optimized for T. heterothallica C1 and synthetized by GenScript (USA), corresponds to positions 4956-2383 of SEQ ID NO: 25. The nucleic acid sequence encoding Leishmania major STT3 gene is also set forth as SEQ ID NO: 11 (LmSTT3 nt). The full amino acid sequence of L. major STT3 is set forth as SEQ ID NO: 12 (LmSTT3 aa). The hypothetical protein (ID2315630) terminator sequence corresponds to positions 2374-1795 of SEQ ID NO: 25. The bgl8 promoter sequence corresponds to positions 9688-8297 of SEQ ID NO: 25. A nucleic acid sequence encoding genomic Trichoderma reesei Mannosidase 1 with artificial HDEL ER-retention signal corresponds to positions 8296-6470 of SEQ ID NO: 25. The nucleic acid sequence encoding T. reesei MNS1 with HDEL-signal is also set forth as SEQ ID NO: 1 (Tr mns1-HDEL nt) and the amino acid sequence as SEQ ID NO: 2 (Tr MNS1-HDEL aa). The ubiquitin-like protein terminator sequence corresponds to positions 6469-5974 of SEQ ID NO: 25. The translation elongation factor 1A promoter sequence corresponds to positions 12584-11528 of SEQ ID NO: 25. The sequence encoding rat GNT2, codon-optimized for M. thermophila C1 and synthetized by GenScript (USA), corresponds to positions 11527-10199 of SEQ ID NO: 25. The nucleic acid sequence encoding rat GNT2 is also set forth as SEQ ID NO: 15 (rat GNT2 nt). The full amino acid sequence of rat GNT2 is set forth as SEQ ID NO: 16 (rat GNT2 aa). The hypothetical protein (ID114107) terminator sequence corresponds to positions 10198-9695 of SEQ ID NO: 25. The alg3 3′flank sequence corresponds to positions 12592-13591 of SEQ ID NO: 25. Fragments were produced by PCR, separated in agarose gel, purified and cloned stepwise using yeast recombination method into the backbone vector pRS426 to get the final 3′arm expression vector pMYT1964 that was verified by sequencing.

To obtain two different empty strains (not expressing a heterologous mammalian glycoprotein) with G1/2 N-glycans, the expression plasmids described above were transformed into a pyr4-minus strain carrying deletions of 14 protease genes and kex2 protease gene under weaker promoter in a single step. In each transformation a pair of one 5′arm expression vector and one 3′arm expression vector (excised from the expression plasmid backbones with MssI) was used. The pairs were: SEQ ID NO: 23 (pMYT1974; alg3 5′flanking fragment−human GNT1 cassette−human GalT1 cassette−C1 gls2a-HDEL cassette−STF cassette−2/3 pyr4)+SEQ ID NO: 24 (pMYT1963) or SEQ ID NO: 25 (pMYT1964) (2/3 pyr4−DR−LmSTT3 cassette−TrMns1-HDEL cassette−rat GNT2 cassette−alg3 3′flanking fragment). The C1 transformation and transformant selection were done essentially as described in WO 2021/094935. The transformation selection was based on functional pyr4 gene. The transformants were screened by PCR to find clones where alg3 gene had been replaced by the constructs. The correct integration of the plasmids/genes to the transformants genome was verified with specific primers. Transformants having the correct integration of the constructs and loss of the alg3 gene were stored as M6589 and M6596 (with ubiquitin-like protein promoter for TrMns1-HDEL), and as M6590 and M6597 (with bgl8 promoter for TrMns1-HDEL), respectively.

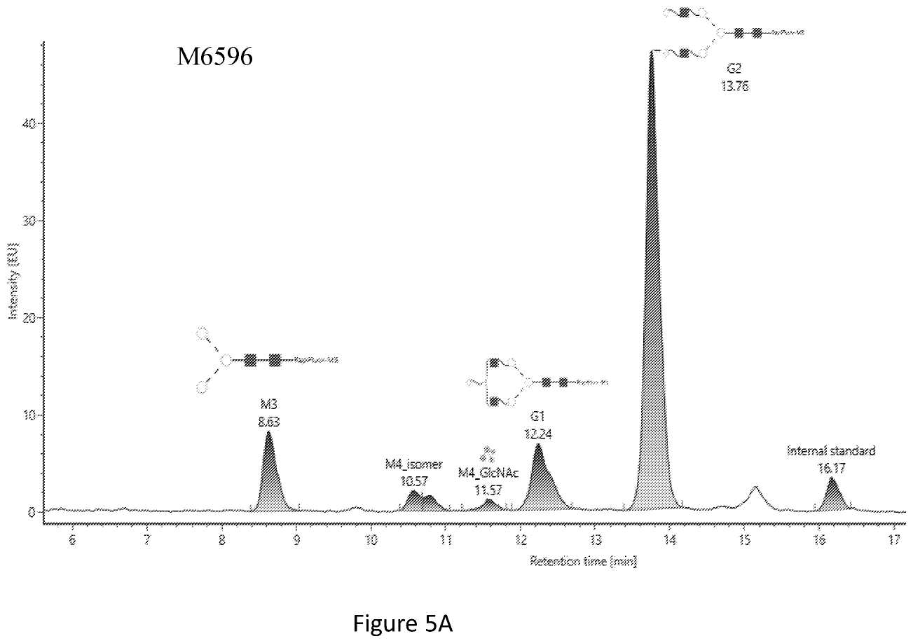

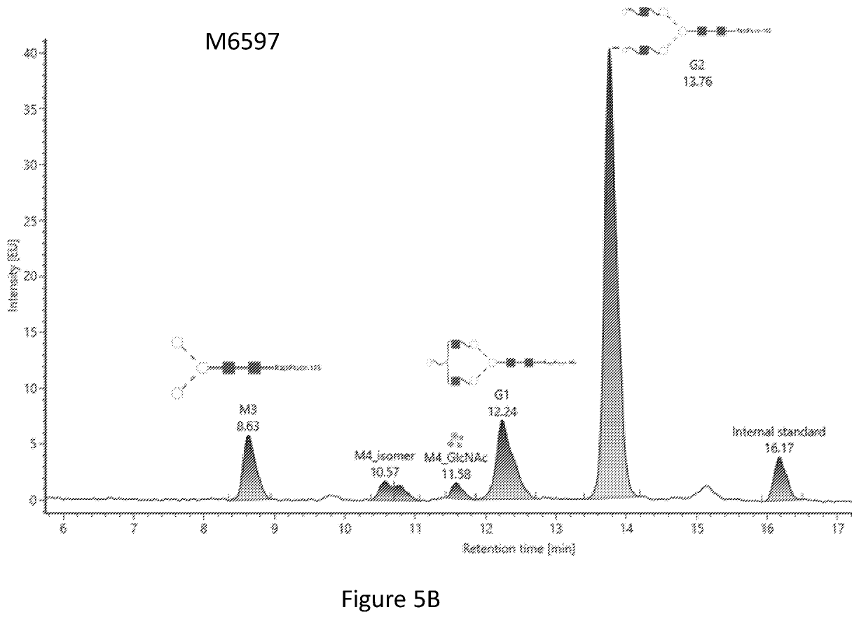

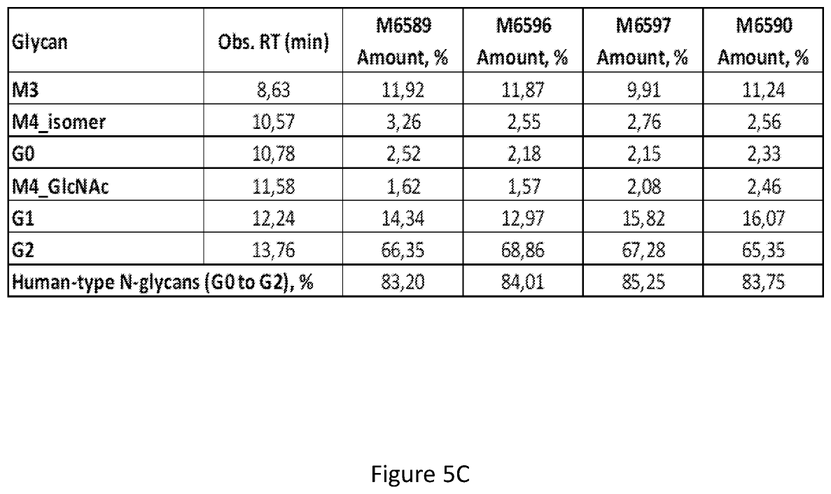

The constructed C1 strains from both transformations were grown in 24-well deep well plates in 3.5 ml of a liquid medium as described for the cultivations of strains in WO 2021/094935. The cultures were carried out at 35° C., 800 RPM, 80% humidity for 4 days. Mycelia were removed by centrifuging and the supernatants from cultivations were subjected to released N-glycan analysis as described in Example 1 of WO 2021/094935. The results are summarized in FIG. 5 for all four strains. FIG. 5 shows that by expressing all six genes affecting glycan structures from alg3 locus the main N-glycans on total secreted proteins are over 80% of human-type (N-glycans that belong to G0 to G2 species) and no detectable Hex6 (GlcNAc₂Man₅Glc) that is thought to block N-glycan modification to G0 and beyond. Also noteworthy is the high amount of final G2 N-glycans. The amount (by %) of human-type N-glycans on total secreted proteins was found to be always lower than what has been observed on any purified target protein like Nivolumab in Example 1.

### Sequences

SEQ ID NO: 1—T. reesei mns1-HDEL nt (coding sequence, i.e. introns removed, 1684 bp)
SEQ ID NO: 2—T. reesei MNS1-HDEL aa (527 aa)
SEQ ID NO: 3—C1 gls2a-HDEL nt (coding sequence, i.e. introns removed, 2961 bp)
SEQ ID NO: 4—C1 GLS2A-HDEL aa (986 aa)
SEQ ID NO: 5—C1 kre2-huGNT1 nt (coding sequence, i.e. introns removed, 1434 bp)
SEQ ID NO: 6: C1 KRE2-huGNT1 aa (477 aa)
SEQ ID NO: 7—C1 rft1 nt (coding sequence, i.e. introns removed, 1839 bp)
SEQ ID NO: 8—C1 RFT1 aa (612 aa)
SEQ ID NO: 9—C1 kre2-boGNT1 nt (coding sequence, i.e. introns removed, 1440 bp)
SEQ ID NO: 10—C1 KRE2-boGNT1 aa (479 aa)
SEQ ID NO: 11—LmSTT3 nt (2574 bp)
SEQ ID NO: 12: LmSTT3 aa (857 aa)
SEQ ID NO: 13—Sckre2-huGalT1 nt (1266 bp)
SEQ ID NO: 14—ScKRE2-huGalT1 aa (421 aa)
SEQ ID NO: 15—rat GNT2 nt (1329 bp)
SEQ ID NO: 16—rat GNT2 aa (442 aa)
SEQ ID NO: 17—pMYT1288 (14648 bp)
SEQ ID NO: 18—-pMYT0721 (13062 bp)
SEQ ID NO: 19—pMYT1451 (15270 bp)
SEQ ID NO: 20—pMYT1452 (15281 bp)
SEQ ID NO: 21—pMYT1453 (18195 bp)
SEQ ID NO: 22—rDNA of Thermothelomyces heterothallica C1
SEQ ID NO: 23—pMYT1974 (20189 bp)
SEQ ID NO: 24—pMYT1963 (18731 bp)
SEQ ID NO: 25—pMYT1964 (19105 bp)

The foregoing description of the specific embodiments will so fully reveal the general nature of the invention that others can, by applying current knowledge, readily modify and/or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. A Thermothelomyces heterothallica genetically modified to produce glycoproteins with mammalian N-glycans, wherein the genetic modification comprises:
(i) deletion or disruption of the alg3 gene such that the Th. heterothallica fails to produce a functional alpha-1,3-mannosyltransferase;
(ii) expression of ER-targeted Mannosidase 1 (alpha-1,2-Mannosidase); and
(iii) expression of ER-targeted Glucosidase 2 alpha-subunit.

2. The Th. heterothallica of claim 1, wherein the Mannosidase 1 is Trichoderma reesei mannosidase.

3. The Th. heterothallica of claim 1, wherein the Glucosidase 2 alpha-subunit is Th. heterothallica Glucosidase 2 alpha-subunit.

4. The Th. heterothallica of claim 1, wherein the genetic modification further comprises expression of heterologous GlcNAc transferase 1 (GNT1) and GlcNAc transferase 2 (GNT2).

5. The Th. heterothallica of claim 4, wherein the heterologous GNT1 and GNT2 are animal-derived.

6. The Th. heterothallica of claim 5, wherein the animal-derived GNT1 is human GNT1.

7. The Th. heterothallica of claim 6, wherein the animal-derived GNT1 is human GNT1 further comprising a Th. heterothallica Golgi localization signal.

8-10. (canceled)

11. The Th. heterothallica of claim 5, wherein the animal-derived GNT1 is bovine GNT1.

12. The Th. heterothallica of claim 11, wherein the animal-derived GNT1 is bovine GNT1 comprising a Th. heterothallica Golgi localization signal.

13-14. (canceled)

15. The Th. heterothallica of claim 5, wherein the animal-derived GNT2 is rat GNT2.

16-17. (canceled)

18. The Th. heterothallica of claim 5, wherein the animal-derived GNT1 is human or bovine GNT1 comprising a Golgi-localization signal from the Th. heterothallica protein KRE2, and the animal-derived GNT2 is rat GNT2.

19. The Th. heterothallica of claim 5, wherein the animal-derived GNT1 is bovine GNT1 comprising a Golgi localization signal from the Th. heterothallica protein KRE2, and the animal-derived GNT2 is rat GNT2.

20. The Th. heterothallica of claim 1, wherein the genetic modification further comprising over-expression of an endogenous flippase or expression of a heterologous flippase.

21-24. (canceled)

25. The Th. heterothallica of claim 1, wherein the genetic modification further comprises expression of the STT3 subunit of a heterologous oligosaccharyltransferase.

26-28. (canceled)

29. The Th. heterothallica of claim 1, wherein the genetic modification further comprises expression of a heterologous galactosyltransferase.

30-33. (canceled)

34. The Th. heterothallica of claim 1, wherein the Th. heterothallica is Th. heterothallica C1, wherein the C1 is a strain modified to delete one or more genes encoding an endogenous protease or chitinase.

35-36. (canceled)

37. The Th. heterothallica of claim 1, further genetically modified to express a heterologous mammalian glycoprotein.

38. (canceled)

39. A method for generating a Th. heterothallica that produces glycoproteins with mammalian N-glycans, comprising:
(a) deleting or disrupting the alg3 gene of the Th. heterothallica such that the Th. heterothallica fails to produce a functional alpha-1,3-mannosyltransferase; and
(b) introducing into the Th. heterothallica: an exogenous polynucleotide encoding ER-targeted Mannosidase 1 (alpha-1,2-Mannosidase) and ER-targeted Glucosidase 2 alpha-subunit.

40. (canceled)

41. A method for producing a glycoprotein with mammalian N-glycans, the method comprising:
(a) providing a Th. heterothallica genetically modified according to claim 1;
(b) culturing the Th. heterothallica under conditions suitable for expressing the glycoprotein; and
(c) recovering the glycoprotein.

42-44. (canceled)

45. A recombinant glycoprotein produced by the Th. heterothallica genetically modified according to claim 1, wherein the glycoprotein comprises GlcNAc₂Man₃GlcNAc₂ (G0) glycans.

46-47. (canceled)


SEQUENCE LISTING (25 sequences)

SEQ ID NO: 1 - DNA, 1584 bp, synthetic construct
  source 1..1584: mol_type = other DNA
atgagattccctagcagctccgtccttgccctcgggctcatcggacctgcgctggcgtatccaaagccgggcgccacaaaacgtggatctcccaaccctacgagggcggcagcagtcaaggccgcattccagacgtcgtggaacgcttaccaccattttgcctttccccatgacgacctccacccggtcagcaacagctttgatgatgagagaaacggctggggctcgtcggcaatcgatggcttggacacggctatcctcatgggggatgccgacattgtgaacacgatccttcagtatgtaccgcagatcaacttcaccacgactgcggttgccaaccaaggcatctccgtgttcgagaccaacattcggtacctcggtggcctgctttctgcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctactgtccggagaagtggtgcatctagcaacaacgtcgctgaaattggaagcctggtgctcgagtggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttgctgccgactcgaccattgcgcatctcgcctctcacccgtcgacgcgcaaggacttgacctttttgtcttcgtacaacggacagtctacgtcgccaaactcaggacatttggccagttttgccggtggcaacttcatcttgggaggcattctcctgaacgagcaaaagtacattgactttggaatcaagcttgccagctcgtactttgccacgtacaaccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttcagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaacggcgggaacaaatttgtctttaacacggaggcgcacccctttagcatccgttcatcatcacgacggggcggccaccttgctcacgacgagctctaa

SEQ ID NO: 2 - protein, 527 aa, synthetic construct
MRFPSSSVLALGLIGPALAYPKPGATKRGSPNPTRAAAVKAAFQTSWNAYHHFAFPHDDLHPVSNSFDDERNGWGSSAIDGLDTAILMGDADIVNTILQYVPQINFTTTAVANQGISVFETNIRYLGGLLSAYDLLRGPFSSLATNQTLVNSLLRQAQTLANGLKVAFTTPSGVPDPTVFFNPTVRRSGASSNNVAEIGSLVLEWTRLSDLTGNPQYAQLAQKGESYLLNPKGSPEAWPGLIGTFVSTSNGTFQDSSGSWSGLMDSFYEYLIKMYLYDPVAFAHYKDRWVLAADSTIAHLASHPSTRKDLTFLSSYNGQSTSPNSGHLASFAGGNFILGGILLNEQKYIDFGIKLASSYFATYNQTASGIGPEGFAWVDSVTGAGGSPPSSQSGFYSSAGFWVTAPYYILRPETLESLYYAYRVTGDSKWQDLAWEAFSAIEDACRAGSAYSSINDVTQANGGGASDDMESFWFAEALKYAYLIFAEESDVQVQANGGNKFVFNTEAHPFSIRSSSRRGGHLAHDEL

SEQ ID NO: 3 - DNA, 2961 bp, synthetic construct
  source 1..2961: mol_type = other DNA
atgcacgtcccaggcccattgtcctcgcggtggacggcctaccttggcctgctgtccgcattttctgcagcatttgcacctgcagtcgccgtcaaggagcatgacttcaaaaagtgccatcaatcgggcttttgtaagcgcaaccgcgaattcgccgaccacgccctcgccacgagctcctgggcgtcaccctacaatgtcgcaccagattccggatcgttcaaagatggccagtaccaggctgtgatcttgaagaccaccaacagcggcgagacggttcggcttcccattaccgtctctttccttgagtctggtacagcgcgagtcacggtggacgaagagaagcgccagaaaggagagattgagttgcgccatggcagcaaagcgaggaaagagcgctataacgaggcagagaaatgggccattgttggagggatgactttggacaaggaggccaaggtcgattatgaggacaagacgcagattaccgtcaagtacgggccaacctccaagttcgaggcaaccatcaaattctctcccttcagcatcgacttcaagcgggacggcatcagccacgtcaaactcaacgaccagggcctccttaacatcgagcactggaggccaaaggttgacaaaccggagcccgagaagaaggacggcgattcgactgaagagaataaggcagaggaagcaaaggaggagcccaagggagaggatgagtctacctggtgggaggagacatttggcggcaacaccgataccaagcctcgcggcccggagagcgtcggtcttgacatctccttcgtcggctacgaacacgtctacggcatcccctcccatgccagctcgctgtcgctgaagcagacccgtggcggcgagggcaactaccaggagccgtacaggatgtacaatgctgacgttttcgagtacatcctcgatagcccaatgactctctacggctctattcctttcatgcaggcccatcgtaaggactcgagcgtgggcgttttctggctgaatgccgcggagacgtgggttgacatcaccaaggcgaaagactccaagaacccgctctctctcggcggcaaggccaggaccaacacgcatacccactggttctccgagagcggtctgctggacgtttttgtcttcctcggcccgacgcccaaggatttgaccgccaggtacggtgagctcactggcaccactgcaatgccccaggagttcgctctcggataccatcagtgccgttggaactacgtctcggacgaggacgtaagggatgtggaccgcaagatggacaagttcaagatgccgtacgacgtcatttggctcgacatcgagtacactgacgagaagaagtactttacctgggacaaacactcgtttacagaccccatcggcatgggcaagcagctggacagtcacggcaggaagctggttaccatcattgatccgcacatcaagaataccgacaattaccccgtcgtggcagagctaaagtccaaggaactcggcgtaaagaacaaagatggcaacctcttcgagggctggtgctggccaggctcgtctcactggatcgacgccttcagtccggctgcgcgcgagtggtggtcgtcgcttttcaagtatgacaaattcaagggcaccatggaaaacacgtggatctggaacgacatgaacgagccgtcggtctttaacggtcccgaaaccacgatgcccaaggacaacctccacgacggaaactgggaacaccgcgacgtgcacaacctgaatggcctgacgttccacaacgcgacctaccatgcgctcctcacccggaagcccggcgagctccgccgcccgttcgtgctgacccggtccttctttgccggctcgcagcgggtcggcgccatgtggaccggcgacaaccaggctgcctgggaccacctcaaggcatcgattcccatggtcctcagccagggtatttcgggcttccccttctcgggcgccgacgtcggcggcttctttggcaacccggagaaggagcttttgacgcggtggtaccaagccggcgccttctaccccttcttccgggggcacgcgcacattgatgcgcgacgccgcgagccatacctcgccggcgagccgtacacgaccatcatcgccgcggcgcttcggctgcgctacagcctgctgccgtcgtggtacactgcgttccggcacgcccatctggacggtacgcccattatcaagcccatgttctacacgcatccgtccgaggaggccggcctcgctatcgaggaccagttcttcgtcggcaacacgggcctgctcgccaagcccgtcacggagaaggagaagacgacggtcgatgtgtggatccccgacggcgaggtctactatgactactttacgtaccaggtcatcccgacagtcaagggcaagacggtcacgctggatgcgccgatggagaagatcccgctgctgatgcgcggcgggcacgtgtttgcgcgccgcgacgtgccgcggcgctcgagcgcgctcatgcgctgggacgactacacgctcgtcgtgacggtgccgcgggaaaacaaggtcgccgagggcgacctgtacgtcgacgacggagactcgtttgagtaccagaacggccagtacatccaccgccggttcgtctacgacggcgcggccaagagcctcagctcggtggatgccgagggccgcgacgcggcctcgatccgggagggcgcgtggatgaagcagatgcgctccgtgggcgtgggcaagatcgtggtggtcggcgcgccggccagctgggcgggcaagaagagcgtcaaggtagagagcgagggcaaggtctgggaggccaggatggagtttacgcctgctgggcagggcagagccgcgttcgcggtgatcaagaaggtgggcgtgaggatcggggcggattggaaggtcgagtttcacgacgagctctga

SEQ ID NO: 4 - protein, 986 aa, synthetic construct
MHVPGPLSSRWTAYLGLLSAFSAAFAPAVAVKEHDFKKCHQSGFCKRNREFADHALATSSWASPYNVAPDSGSFKDGQYQAVILKTTNSGETVRLPITVSFLESGTARVTVDEEKRQKGEIELRHGSKARKERYNEAEKWAIVGGMTLDKEAKVDYEDKTQITVKYGPTSKFEATIKFSPFSIDFKRDGISHVKLNDQGLLNIEHWRPKVDKPEPEKKDGDSTEENKAEEAKEEPKGEDESTWWEETFGGNTDTKPRGPESVGLDISFVGYEHVYGIPSHASSLSLKQTRGGEGNYQEPYRMYNADVFEYILDSPMTLYGSIPFMQAHRKDSSVGVFWLNAAETWVDITKAKDSKNPLSLGGKARTNTHTHWFSESGLLDVFVFLGPTPKDLTARYGELTGTTAMPQEFALGYHQCRWNYVSDEDVRDVDRKMDKFKMPYDVIWLDIEYTDEKKYFTWDKHSFTDPIGMGKQLDSHGRKLVTIIDPHIKNTDNYPVVAELKSKELGVKNKDGNLFEGWCWPGSSHWIDAFSPAAREWWSSLFKYDKFKGTMENTWIWNDMNEPSVFNGPETTMPKDNLHDGNWEHRDVHNLNGLTFHNATYHALLTRKPGELRRPFVLTRSFFAGSQRVGAMWTGDNQAAWDHLKASIPMVLSQGISGFPFSGADVGGFFGNPEKELLTRWYQAGAFYPFFRGHAHIDARRREPYLAGEPYTTIIAAALRLRYSLLPSWYTAFRHAHLDGTPIIKPMFYTHPSEEAGLAIEDQFFVGNTGLLAKPVTEKEKTTVDVWIPDGEVYYDYFTYQVIPTVKGKTVTLDAPMEKIPLLMRGGHVFARRDVPRRSSALMRWDDYTLVVTVPRENKVAEGDLYVDDGDSFEYQNGQYIHRRFVYDGAAKSLSSVDAEGRDAASIREGAWMKQMRSVGVGKIVVVGAPASWAGKKSVKVESEGKVWEARMEFTPAGQGRAAFAVIKKVGVRIGADWKVEFHDEL

SEQ ID NO: 5 - DNA, 1434 bp, synthetic construct
  source 1..1434: mol_type = other DNA
atggcggttgctcgccccgtgagggcgctggcgctgacgggcgttgtcctgtggtgtttctttgtatggcagcttttcaagccaacaggatcgcccaagagcccggccagggtccctctcctgtcgagagagcgcgatcctaatctagaccccaccggcgagcccgagggtgttctgacatatgcgtccggggagtacgcacccggcccctccgtcagcgccctcgacggcgaccccgcctccctgacccgcgaggtcatccgcctcgcccaggacgccgaggtcgagctcgagcgccagcgcggcctgctccagcagatcggcgacgccctctcctcgcagcgcggccgcgtccccaccgccgcccctcccgcccagccgcgcgtccccgtcaccccggcccccgccgtcatccccatcctggtcatcgcctgcgaccgcagcaccgtccgcaggtgcctggacaagctcctgcactaccgcccctcggccgagctcttccccatcatcgtctcgcaggactgcggccacgaggagaccgcccaggccatcgcctcctacggctcggccgtcacccacatccgccagcccgacctctcctccatcgccgtcccgcccgaccaccgcaagttccagggctactacaagatcgcccgccactaccgctgggccctgggccaggtcttccgccagttccgcttccccgccgccgtcgtcgtcgaggacgacctggaggtcgcccccgacttcttcgagtacttccgcgccacctaccccctgctcaaggccgaccccagcctgtggtgcgtctccgcctggaacgacaacggcaaggagcagatggtcgacgcctcccgccccgagctgctctaccgcaccgacttcttcccgggcctgggctggctgctcctggccgagctgtgggccgagctggagcccaagtggcccaaggccttctgggacgactggatgcgcaggcccgagcagcgccagggccgcgcctgcatccgccccgagatctcgcgcaccatgaccttcggccgcaagggcgtctcccacggccagttcttcgaccagcacctcaagttcatcaagctgaaccagcagttcgtccacttcacccagctcgacctgtcctacctgcagcgcgaggcctacgaccgcgacttcctggcccgcgtctacggcgccccgcagctccaggtcgagaaggtccgcaccaacgaccgcaaggagctgggcgaggtccgcgtccagtacaccggccgcgactccttcaaggccttcgccaaggccctcggcgtcatggacgacctgaagtcgggcgtcccccgcgccggctaccgcggcatcgtcaccttccagttccgcggccgcagggtccacctggcccctccgcccacctgggagggctacgaccccagctggaactaa

SEQ ID NO: 6 - protein, 477 aa, synthetic construct
MAVARPVRALALTGVVLWCFFVWQLFKPTGSPKSPARVPLLSRERDPNLDPTGEPEGVLTYASGEYAPGPSVSALDGDPASLTREVIRLAQDAEVELERQRGLLQQIGDALSSQRGRVPTAAPPAQPRVPVTPAPAVIPILVIACDRSTVRRCLDKLLHYRPSAELFPIIVSQDCGHEETAQAIASYGSAVTHIRQPDLSSIAVPPDHRKFQGYYKIARHYRWALGQVFRQFRFPAAVVVEDDLEVAPDFFEYFRATYPLLKADPSLWCVSAWNDNGKEQMVDASRPELLYRTDFFPGLGWLLLAELWAELEPKWPKAFWDDWMRRPEQRQGRACIRPEISRTMTFGRKGVSHGQFFDQHLKFIKLNQQFVHFTQLDLSYLQREAYDRDFLARVYGAPQLQVEKVRTNDRKELGEVRVQYTGRDSFKAFAKALGVMDDLKSGVPRAGYRGIVTFQFRGRRVHLAPPPTWEGYDPSWN

SEQ ID NO: 7 - DNA, 1838 bp, synthetic construct
  source 1..1838: mol_type = other DNA
atggcggcgacagagaaaacgggcagcagcagcacgcgagcgctgcgaggcgcgtccctgctgatcgtgctgcaagtggtctcgcgcgccatcacttttgtggccaaccaggtgctgctgcgatttctgactgcccagcttctgggggtgtcgacgcagctcgaagtctactacctgtccgtcatcttctttgcccgggagagcctgagggttgccatccagagacaggacctgacaggcctcccggacaagaaggacaacaaacagaatggccatgtcaaccaatctgcccaggccgtagtcaacctgggctatctcgcccttgccctgggcgttccgctcgcatttctctttgggtggctctatctcgactctctgagcgcaagcaccctgctatccgcgcccaacttggtcgtttccctctacatctacgccgtcgccgctgtcctggagctcgcctcggaacccgccttcgtcgtgatgcagactcgactgcagttcggcactcgtgcagctggcgaatcggttgccacgtttctgcgctgcatagtcacgctcggaacagcagcctggggagccggtcgcgatctgggcgtcctgccgtttgccctgggccagttgagctacgggcttgggctgctcgcagtctacacctggcacggggccgcgctggcaaggagggaaggcttctcccttcttcctctccgcatcaccgctccttatagcacgacagcaccacctcaacaaccatcgggcaaatccgtcttcgtcctctcctacttctaccgccctaccctgcagctcgcctccagcatgatggcccagagcgtgtgaagcacatactcacccagggtgacacgttcctcgtcagcatcttgtccacgcccacggcccagggggtgtatgccctcgccaacaactacggcggccttgtcgcccgcctggtcttccagcccatcgaggaaagcagtcgcagctacttcagccgccttctcgccccttccgatgctgctgctgccgctgctgccgctgccgccgccgccgccacagaagcgtccccagcaagacagtcttctccggaagccagatcgaaaccccaatccgagaccgagaccgagccagcagcggaagcagcggaagcagcagcagcagcacggggggcagtctctcgggccaaaacagctctccagtccctcctcaaatcctacctcctcctctccctcgtcgtcacggccctcggcccgaccgccgccggccccctgctctccctcgtcgccggccgccgctgggccggctccggcgcgggcgcctgcctggccgcctacgcgtggtacgtcccgctgctggccatcaacggcgtggccgaggcctttgtggcctcggtcgcgaccgaggccgaggtccaccggcagagcgcatggatggccgccttctcagtcgccttcgccgccgccggcttcgtcttcctgcggctactgggctggggcgccgtggggctggtcgttgccaacggcatcaacatggcctgccgcatcgcctggtgtgccgttttcataagccgatatttcgccgcgaaggggcaccgcttcgacctgctcgacgtcatgccctcgcccgcgtccgtgctggcgggcgcggttgcctctcaggtcgtccggaagctcgttcgctcggcagcatcagaggaggaggtcccacttggcgtgaaggaggccgtgatggacctggtcagagtcgcctcggttgccctgccgtttgttcttgtcgtggccgtgtccgaacggcggttcttgtacggcgtgttccaggctcttcggggaacccgaggggctccatga

SEQ ID NO: 8 - protein, 612 aa, Thermothelomyces heterothallica
MAATEKTGSSSTRALRGASLLIVLQVVSRAITFVANQVLLRFLTAQLLGVSTQLEVYYLSVIFFARESLRVAIQRQDLTGLPDKKDNKQNGHVNQSAQAVVNLGYLALALGVPLAFLFGWLYLDSLSASTLLSAPNLVVSLYIYAVAAVLELASEPAFVVMQTRLQFGTRAAGESVATFLRCIVTLGTAAWGAGRDLGVLPFALGQLSYGLGLLAVYTWHGAALARREGFSLLPLRITAPYSTTAPPQQPSGKSVFVLSYFYRPTLQLASSMMAQSVVKHILTQGDTFLVSILSTPTAQGVYALANNYGGLVARLVFQPIEESSRSYFSRLLAPSDAAAAAAAAAAAAATEASPARQSSPEARSKPQSETETEPAAEAAEAAAAARGAVSRAKTALQSLLKSYLLLSLVVTALGPTAAGPLLSLVAGRRWAGSGAGACLAAYAWYVPLLAINGVAEAFVASVATEAEVHRQSAWMAAFSVAFAAAGFVFLRLLGWGAVGLVVANGINMACRIAWCAVFISRYFAAKGHRFDLLDVMPSPASVLAGAVASQVVRKLVRSAASEEEVPLGVKEAVMDLVRVASVALPFVLVVAVSERRFLYGVFQALRGTRGAP

SEQ ID NO: 9 - DNA, 1440 bp, synthetic construct
  source 1..1440: mol_type = other DNA
atggcggttgctcgccccgtgagggcgctggcgctgacgggcgttgtcctgtggtgtttctttgtatggcagcttttcaagccaacaggatcgcccaagagcccggccagggtccctctcctgtcgagagagcgcgatcctaatctagaccccaccggcgagcccgagggtgttctgacatatgcgtccggggagtacgcacccggcccgtccgacagcgccctggacgacgaccccgccagcctcacccgcgaggtcatccgcctggcccaggacgcggaggtcgagctggagcgccagcgcggcctgctccagcagatccgcgagcaccacgcccgctggtcgcagcgctggcgcgtccccaccgtcgcccctcccgtcccgccccgcgtccccgtcaccaccccgcccgccgtcatcccgatcctggtcatcgcctgcgaccgctccaccgtccgcaggtgcctggacaagctcctgaactaccgccccagcgccgagcacttcccgatcatcgtctcccaggactgcggccacgaggagaccgcccaggtcatcgcctcctacggctcggccgtcatgcacatccgccagccggacctcagcaccatcgccgtcccgcccgaccaccgcaagttccagggctactacaagatcgcccgccactaccgctgggccctgggccaggtcttccacgagttcaagttcccggccgccgtcgtcgtcgaggacgacctggaggtcgccccggacttcttcgagtacttccaggccacctaccccctcctgcgcgccgacccgtcgctctggtgcgtctccgcctggaacgacaacggcaaggagcagatggtcgacagctcgaagcccgagctcctgtaccgcaccgacttcttccccggcctgggctggctcctgctggccgagctctgggccgagctggagcccaagtggcccaaggccttctgggacgactggatgcgcaggccggagcagcgccagggcagggcctgcgtccgcccggagatctcgcgcaccatgaccttcggccgcaagggcgtcagccacggccagttcttcgaccagcacctgaagttcatcaagctcaaccagcacttcgtccccttcacccagctggacctctcgtacctccgccaggagacctacgaccgcgacttcctggcccgcgtctacggcgccccgctgctccaggtcgagaaggtccgcacctcggagcgctccgagctccaggaagtccgcgtccagtacacctcgcgcgacagcttcaaggccttcgccaaggccctgggcgtcatggacgacctcaagtccggcgtccccagggccggctaccgcggcatcgtctccttcctctaccgcggcaggcgcgtccacctggccccgccccagacctgggacggctacgacccgagctggaactaa

SEQ ID NO: 10 - protein, 479 aa, synthetic construct
MAVARPVRALALTGVVLWCFFVWQLFKPTGSPKSPARVPLLSRERDPNLDPTGEPEGVLTYASGEYAPGPSDSALDDDPASLTREVIRLAQDAEVELERQRGLLQQIREHHARWSQRWRVPTVAPPVPPRVPVTTPPAVIPILVIACDRSTVRRCLDKLLNYRPSAEHFPIIVSQDCGHEETAQVIASYGSAVMHIRQPDLSTIAVPPDHRKFQGYYKIARHYRWALGQVFHEFKFPAAVVVEDDLEVAPDFFEYFQATYPLLRADPSLWCVSAWNDNGKEQMVDSSKPELLYRTDFFPGLGWLLLAELWAELEPKWPKAFWDDWMRRPEQRQGRACVRPEISRTMTFGRKGVSHGQFFDQHLKFIKLNQHFVPFTQLDLSYLRQETYDRDFLARVYGAPLLQVEKVRTSERSELQEVRVQYTSRDSFKAFAKALGVMDDLKSGVPRAGYRGIVSFLYRGRRVHLAPPQTWDGYDPSWN

SEQ ID NO: 11 - DNA, 2574 bp, synthetic construct
  source 1..2574: mol_type = other DNA
atgggcaagcgcaagggcaactccctgggcgactccggctccgccgccaccgccagccgcgaggcctccgcccaggccgaggacgccgcctcccagaccaagaccgccagcccgcccgccaaggtcatcctcctgcccaagaccctgaccgacgagaaggacttcatcggcatcttccccttcccgttctggccggtccacttcgtcctgaccgtcgtcgccctcttcgtcctggccgcctcctgcttccaggccttcaccgtccgcatgatctccgtccagatctacggctacctcatccacgagttcgacccctggttcaactaccgcgccgccgagtacatgagcacccacggctggtcggccttcttctcctggttcgactacatgtcctggtacccgctcggcaggcccgtcggcagcaccacctacccgggcctccagctgaccgccgtcgccatccaccgcgccctggccgccgccggcatgcccatgtcgctgaacaacgtctgcgtcctgatgcccgcctggttcggcgccatcgccaccgccaccctcgccttctgcacctacgaggcctcgggctccaccgtcgccgccgccgccgccgccctgtccttcagcatcatcccggcccacctcatgcgctccatggccggcgagttcgacaacgagtgcatcgccgtcgccgccatgctcctgaccttctactgctgggtccgctccctccgcacccgcagctcctggccgatcggcgtcctgaccggcgtcgcctacggctacatggccgccgcctggggcggctacatcttcgtcctgaacatggtcgccatgcacgccggcatctccagcatggtcgactgggcccgcaacacctacaaccccagcctcctgcgcgcctacaccctcttctacgtcgtcggcaccgccatcgccgtctgcgtcccgcccgtcggcatgagcccgttcaagtccctggagcagctgggcgccctcctggtcctcgtcttcctgtgcggcctccaggtctgcgaggtcctccgcgcccgcgccggcgtcgaggtccgctcccgcgccaacttcaagatccgcgtccgcgtcttcagcgtcatggccggcgtcgccgccctggccatctcggtcctcgcccccaccggctacttcggccccctgagcgtccgcgtccgcgccctcttcgtcgagcacacccgcaccggcaacccgctcgtcgactccgtcgccgagcaccagccggcctcgcccgaggccatgtgggccttcctccacgtctgcggcgtcacctggggcctgggcagcatcgtcctggccgtctcgaccttcgtccactactcgccctccaaggtcttctggctcctgaactcgggcgccgtctactacttctccacccgcatggcccgcctcctgctcctgtcgggccccgccgcctgcctgtcgaccggcatcttcgtcggcaccatcctggaggccgccgtccagctctccttctgggacagcgacgccaccaaggccaagaagcagcagaagcaggcccagcgccaccagaggggcgccggcaagggctcgggccgcgacgacgccaagaacgccaccaccgcccgcgccttctgcgacgtcttcgccggctcctccctggcctggggccaccgcatggtcctgtccatcgccatgtgggccctggtcaccaccaccgccgtcagcttcttcagctcggagttcgccagccactcgaccaagttcgccgagcagtccagcaaccccatgatcgtcttcgccgccgtcgtccagaaccgcgccaccggcaagccgatgaacctgctcgtcgacgactacctgaaggcctacgagtggctgcgcgacagcaccccggaggacgcccgcgtcctcgcctggtgggactacggctaccagatcaccggcatcggcaaccgcacctcgctggccgacggcaacacctggaaccacgagcacatcgccaccatcggcaagatgctcaccagcccggtcgtcgaggcccacagcctggtccgccacatggccgactacgtcctcatctgggccggccagtcgggcgacctgatgaagagcccgcacatggcccgcatcggcaactccgtctaccacgacatctgcccggacgacccgctgtgccagcagttcggcttccaccgcaacgactacagccgccccaccccgatgatgcgcgcctccctcctgtacaacctgcacgaggccggcaagcgcaagggcgtcaaggtcaacccctcgctcttccaggaagtctactcctccaagtacggcctggtccgcatcttcaaggtcatgaacgtctccgccgagagcaagaagtgggtcgccgacccggccaaccgcgtctgccacccgcccggctcctggatctgcccgggccagtacccgcccgccaaggagatccaggagatgctggcccaccgcgtccccttcgaccaggtcaccaacgccgaccgcaagaacaacgtcggcagctaccaggaggagtacatgcgccggatgcgcgagtcggagaaccgccgctaa

SEQ ID NO: 12 - protein, 857 aa, synthetic construct
MGKRKGNSLGDSGSAATASREASAQAEDAASQTKTASPPAKVILLPKTLTDEKDFIGIFPFPFWPVHFVLTVVALFVLAASCFQAFTVRMISVQIYGYLIHEFDPWFNYRAAEYMSTHGWSAFFSWFDYMSWYPLGRPVGSTTYPGLQLTAVAIHRALAAAGMPMSLNNVCVLMPAWFGAIATATLAFCTYEASGSTVAAAAAALSFSIIPAHLMRSMAGEFDNECIAVAAMLLTFYCWVRSLRTRSSWPIGVLTGVAYGYMAAAWGGYIFVLNMVAMHAGISSMVDWARNTYNPSLLRAYTLFYVVGTAIAVCVPPVGMSPFKSLEQLGALLVLVFLCGLQVCEVLRARAGVEVRSRANFKIRVRVFSVMAGVAALAISVLAPTGYFGPLSVRVRALFVEHTRTGNPLVDSVAEHQPASPEAMWAFLHVCGVTWGLGSIVLAVSTFVHYSPSKVFWLLNSGAVYYFSTRMARLLLLSGPAACLSTGIFVGTILEAAVQLSFWDSDATKAKKQQKQAQRHQRGAGKGSGRDDAKNATTARAFCDVFAGSSLAWGHRMVLSIAMWALVTTTAVSFFSSEFASHSTKFAEQSSNPMIVFAAVVQNRATGKPMNLLVDDYLKAYEWLRDSTPEDARVLAWWDYGYQITGIGNRTSLADGNTWNHEHIATIGKMLTSPVVEAHSLVRHMADYVLIWAGQSGDLMKSPHMARIGNSVYHDICPDDPLCQQFGFHRNDYSRPTPMMRASLLYNLHEAGKRKGVKVNPSLFQEVYSSKYGLVRIFKVMNVSAESKKWVADPANRVCHPPGSWICPGQYPPAKEIQEMLAHRVPFDQVTNADRKNNVGSYQEEYMRRMRESENRR

SEQ ID NO: 13 - DNA, 1266 bp, synthetic construct
  source 1..1266: mol_type = other DNA
atggccctcttcctgagcaagcgcctcctgcgcttcaccgtcatcgccggcgccgtcatcgtcctcctgctcaccctcaacagcaactcgcgcacccagcagtacatcccgagctcgatctcggccgccttcgacttcacctcgggcagcatctccccggagcagcaggtcatctcggaggagaacgacgccaagaagctcgagcagtccgccctgaactccgaggcctcggaggactcggaggccatggacgaggagtccaaggccctcaaggccgccgccgagaaggccgacgccccccgaaccggcggcgcccgccctccgcctcctctgggcgccagcagccagcctcgccctggcggcgacagcagccccgtcgtcgacagcggccctggccctgccagcaacctcaccagcgtccccgtcccccacaccaccgccctcagcctccccgcctgccccgaggagagccccctcctcgtcggccccatgctcatcgagttcaacatgcccgtcgacctcgagctggtcgccaagcagaaccccaacgtcaagatgggcggcaggtacgcccctcgcgactgcgtcagcccccacaaggtcgccatcatcatcccgttccgcaaccgccaggagcacctcaagtactggctctactacctccaccccgtcctccagcgccagcagctcgactacggcatctacgtcatcaaccaggccggcgacaccatcttcaaccgcgccaagctcctcaacgtcggcttccaggaggccctcaaggactacgactacacgtgcttcgtctttagcgacgtcgacctcatccccatgaacgaccacaacgcctaccgctgcttcagccagccccgccacatcagcgtcgccatggacaagttcggcttcagcctcccctacgtccagtacttcggcggcgtcagcgccctcagcaagcagcagttcctcaccatcaacggcttccccaacaactactggggctggggcggcgaggacgacgacatctttaaccgcctcgtctttcgcggcatgagcatcagccgccccaacgccgtcgtcggccgctgccgcatgatccgccacagccgcgacaagaagaacgagcccaacccccagcgcttcgaccgaatcgcccacaccaaggagaccatgctcagcgacggcctcaacagcctcacctaccaggtcctcgacgtccagcgctaccccctctacacccagatcaccgtcgacatcggcacccccagctaa

SEQ ID NO: 14 - protein, 421 aa, synthetic construct
MALFLSKRLLRFTVIAGAVIVLLLTLNSNSRTQQYIPSSISAAFDFTSGSISPEQQVISEENDAKKLEQSALNSEASEDSEAMDEESKALKAAAEKADAPRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 15 - DNA, 1329 bp, synthetic construct
  source 1..1329: mol_type = other DNA
atgcgcttccgcatctacaagcgcaaggtcctcatcctgaccctggtcgtcgccgcctgcggcttcgtcctctggagctccaacggccgccagcgcaagaacgacgccctcgccccgcccctcctggactccgagccgctgaggggcgccggccacttcgccgccagcgtcggcatccgcagggtctcgaacgactcggccgccccgctcgtccccgccgtcccccgcccggaggtcgacaacctgaccctccgctaccgcagcctcgtctaccagctgaacttcgaccagatgctccgcaacgtcgacaaggacggcacctggtccccgggcgagctggtcctcgtcgtccaggtccacaaccgcccggagtacctccgcctcctgatcgactcgctccgcaaggcccagggcatccgcgaggtcctcgtcatcttctcccacgacttctggagcgccgagatcaactcgctcatctccagcgtcgacttctgccccgtcctgcaggtcttcttcccgttcagcatccagctctacccctccgagttccccggctcggacccccgcgactgcccccgcgacctcaagaagaacgccgccctgaagctcggctgcatcaacgccgagtaccccgacagcttcggccactaccgcgaggccaagttctcgcagaccaagcaccactggtggtggaagctgcacttcgtctgggagcgcgtcaaggtcctccaggactacaccggcctgatcctcttcctggaggaagaccactacctggcccccgacttctaccacgtcttcaagaagatgtggaagctcaagcagcaggagtgccccggctgcgacgtcctctccctgggcacctacaccaccatccgctccttctacggcatcgccgacaaggtcgacgtcaagacctggaagtcgaccgagcacaacatgggcctggccctgacccgcgacgcctaccagaagctgatcgagtgcaccgacaccttctgcacctacgacgactacaactgggactggaccctccagtacctcaccctggcctgcctccccaaggtctggaaggtcctggtcccgcaggcccccaggatcttccacgccggcgactgcggcatgcaccacaagaagacctgccgccccagcacccagtccgcccagatcgagagcctcctgaacaacaacaagcagtacctcttccccgagaccctggtcatcggcgagaagttccccatggccgccatctcgcccccgcgcaagaacggtggctggggcgacatccgcgaccacgagctctgcaagtcctaccgcaggctgcagtaa

SEQ ID NO: 16 - protein, 442 aa, synthetic construct
MRFRIYKRKVLILTLVVAACGFVLWSSNGRQRKNDALAPPLLDSEPLRGAGHFAASVGIRRVSNDSAAPLVPAVPRPEVDNLTLRYRSLVYQLNFDQMLRNVDKDGTWSPGELVLVVQVHNRPEYLRLLIDSLRKAQGIREVLVIFSHDFWSAEINSLISSVDFCPVLQVFFPFSIQLYPSEFPGSDPRDCPRDLKKNAALKLGCINAEYPDSFGHYREAKFSQTKHHWWWKLHFVWERVKVLQDYTGLILFLEEDHYLAPDFYHVFKKMWKLKQQECPGCDVLSLGTYTTIRSFYGIADKVDVKTWKSTEHNMGLALTRDAYQKLIECTDTFCTYDDYNWDWTLQYLTLACLPKVWKVLVPQAPRIFHAGDCGMHHKKTCRPSTQSAQIESLLNNNKQYLFPETLVIGEKFPMAAISPPRKNGGWGDIRDHELCKSYRRLQ

SEQ ID NO: 17 - DNA, 14648 bp, synthetic construct
  source 1..14648: mol_type = other DNA
gtttaaaccaacatgcccgagacgtctaccaatataacctggcccacgcggacgacgaacggacggcgatagccactgtcgaaccggttcaacagagtaaatgcgcagtacttattagagtaaatgcgcagtacttattcgtgcttgtatgtactacattaaccagcagcaacatcggcaggccgacaatgtattagctgcggcttgggattggcggaagtgtcgagcgtcggcaccttttcgccgaaaattattagaagcacggcagccactttcccgcctgcaggaaaccccgcaacagttcctgcccgagacatctaggcatgttcatcagtcgtacgaattcactttcgttatccatttcctctcagcgatctttaactctttgtgcttactcagctcccaagcccaatgccccggcgaagccttgcactgctcaccaggcagatggcgccctaacaagccatcaccacgcgctgctgatgtcgatcgttcagcctgcacagcaggggggcagcaccagagaggggcaatgcgaggagggatctggcgaggaaggtgacattcttctgcaggaaccgcaccaacgcctttgcacccctgcaagtcaccggaagcacgacagcacactacacagtacaagtaggcattgcgagcagtccagtccgctttccagtcctccagagcgccgtgaaaggcaagttcaagtagtcatactgaacgcatgctcgtcgtcggcattcgccccggtccggctcgcttagcgggagacggcgcgctcggctaacgacaagtatgggagggccccggccatgagatttcgagttgcctgaaatggctgacaacgccgccgcggtcaaaggactaataaataagttgcgtggttgagactacactactagtatgtactgagtatgcagaagcggagctcagaaggccgaacaagaccgcaacggttcaacgaaggatttcgcgtcagcggtgccagggcgaagtactccgtactcggagtactgtgtagtgtgcgcatggaagagcaacactcaaattgtcgagctgagaaggcgccctccgagacatcggcaaatataaagtgccgtcatggcgccagatcggccttcgcgcgagctctcgccgatccatccctcttggtacggaccgcggtcggtgttgagtgccgttctttcattcacccgccggcggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgagattccctagcagctccgtccttgccctcgggctcatcggacctgcgctggcgtatccaaagccgggcgccacaaaacgtggatctcccaaccctacgagggcggcagcagtcaaggccgcattccagacgtcgtggaacgcttaccaccattttgcctttccccatgacgacctccacccggtcagcaacagctttgatgatgagaggtaagctgcgagctcgtgacggaacctcagtcctttcataaagtggcgaatttgtatcttactttggcctagaaacggctggggctcgtcggcaatcgatggcttggacacggctatcctcatgggggatgccgacattgtgaacacgatccttcagtatgtaccgcagatcaacttcaccacgactgcggttgccaaccaaggcatctccgtgttcgagaccaacattcggtacctcggtggcctgctttctggtacgtgttcgatgggcgagcctagtttccgcgtcaaggattgttcggcgctgacggtatggctaatcgatgattctacctagcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctactgtccggagaagtggtgcatctagcaacaacgtcgctgaaattggaagcctggtgctcgagtggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttgctgccgactcgaccattgcgcatctcgcctctcacccgtcgacgcgcaaggacttgacctttttgtcttcgtacaacggacagtctacgtcgccaaactcaggacattgtaagtttctccattcgacagtcatctcgagacgtcgtacgtggtggtgattgtgtgctaacagtctcccccttttcttgccttgtagtggccagttttgccggtggcaacttcatcttgggaggcattctcctgaacgagcaaaagtacattgactttggaatcaagcttgccagctcgtactttgccacgtacaaccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttcagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaacggcgggaacaaatttgtctttaacacggaggcgcacccctttagcatccgttcatcatcacgacggggcggccaccttgctcacgacgagctctaaggcaggcaggagttggagtatgagggtagccgctgatggctattcttcccacgtttttgtgtgtttcctcttcatttttttttctcttgccgcaacatgacggctcctgtctctgaagggaacccctgaaattcagggttatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtcgccggcgttaattaacccaaagtaataagtctgtagtaattggtctcgccctgaattccaaactataaatcaaccactttccctcctcccccccgcccccacttggtcgattcttcgttttctctctaccttctttctattcggttttcttcttcttttattttccctctcccatcaatcaaattcatatttgaaaaaaattaacaatggagtccacacccacgaaacaaaaagctattttttctgcctcgctccttctgttcgccgaacgcgggtttgacgccactacgatgccgatgatcgctgaaaatgctaaggtcggcgcaggaacgatttaccgatactttaagaataaggagagtctggtcaacgagctgttccagcagcacgttaatgaatttttgcaatgtatcgagagtggcttggcgaacgaaagggacggttatcgcgatgggttccatcatatcttcgagggaatggtcacattcacaaagaaccatccgcgcgccttgggatttatcaagacacattcccaaggtacattcctaaccgaagagtcacgccttgcataccaaaaacttgttgagttcgtctgcaccttctttcgagagggacagaaacagggcgtaattcgaaacttgcccgagaatgccctgatcgccatcctattcggatcgtttatggaggtctatgagatgatcgaaaacgattatctctctctaacggatgagttgcttacgggggtagaggaatcgctctgggctgctctctcccgacaatcggctagccctcccaagaagaagcgcaaggtcagcacggccccccccacggacgtctccctcggcgacgagctccacctggacggcgaggacgtcgccatggcccacgccgacgccctcgacgacttcgacctcgacatgctgggcgacggcgacagccccggccccggctttaccccccacgactccgccccctacggcgccctggacatggccgacttcgagtttgagcagatgttcaccgacgccctgggcattgacgagtacggcggctgaggccggccgcgatacccatcatcaacacctgatgttctggggtccctcgtgaggtttctccaggtgggcaccaccatgcgctcacttctacgacgaaacgatcaatgttgctatgcatgagcactcgactatgaatcgaggcacgttaattgagaggctgggaataagggttccatcagaacttctctgggaatgcaaaacaaaagggaacaaaaaaactagatagaagtgaattcatgacttcgacaaccaaatcatcttgtctccgtctgcatacgtgaagcttgtgacgattattctcgcgatgccacgacaaaggttgtgcgaccgtatcttgtccactgtcgtccagtctgcctattccccctccagtgctgccatgtgtcgtaccttgaggtaggtagtctacctaggccagggagctgttagtgcccggctactgggtaatttgtagcgctggagcgattcggtcacaggcgtcaagagtgctgtagcaatgtccgacgccattgatcctgatatcaaataccacctgggcaggtctgggtatgtgaggtcttgtcggatgtgtcgagttcttctccaacgtagtgttcattcgcgctcatttaaatcggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaagcggccgcggcgcgccctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcggtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 18 - DNA, 13062 bp, synthetic construct
  source 1..13062: mol_type = other DNA
ggcgcgccgtttaaaccccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtggcgcgcctgctatgcatgagcactcgactatgaatcgaggcacgttaattgagaggctgggaataagggttccatcagaacttctctgggaatgcaaaacaaaagggaacaaaaaaactagatagaagtgaattcatgacttcgacaaccaaatcatcttgtctccgtctgcatacgtgaagcttgtgacgattattctcgcgatgccacgacaaaggttgtgcgaccgtatcttgtccactgtcgtccagtctgcctattccccctccagtgctgccatgtgtcgtaccttgaggtaggtagtctacctaggccagggagctgttagtgcccggctactgggtaatttgtagcgctggagcgattcggtcacaggcgtcaagagtgctgtagcaatgtccgacgccattgatcctgatatcaaataccacctgggcaggtctgggtatgtgaggtcttgtcggatgtgtcgagttcttctccaacgtagtgttcattcgcgctcatgcggccgcagattcatcaactcattgctggagttagcatatctacaattgggtgaaatggggagcgatttgcaggcatttgctcggctagtcggaatgaacattcattccgagacctaggatgtgacggaatgaaggttcattccggactctagataagcacggaatgaactttcattccgctgaagcttgtcaatcggaatgaaggttcattccggctagtcggaatgaacattcattccgagacctaggatgtgacggaatgaaggttcattccggactctagataagcacggaatgaactttcattccgctgaagcttgtcaatcggaatgaaggttcattccggctagttctccccggaaactgtggccatatgttcaaagactaggatggataaatggggtatataaagcaccctgactcccttcctccaagttctatctaaccagccatcctacactctacatatccacaccaatctactacaattacaaccataatgcacgtcccaggcccattgtcctcgcggtggacggcctaccttggcctgctgtccgcattttctgcagcatttgcacctgcaggtgggtcccgcgagcatctgatgccaccagacgaccgagttgacgatagtagtcgccgtcaaggagcatgacttcaaaaagtgccatcaatcgggcttttgtaagcgcaaccgcgaattcgccgaccacgccctcgccacgagctcctgggcgtcaccctacaatgtcgcaccagattccggatcgttcaaagatggccagtaccaggctgtgatcttgaagaccaccaacagcggcgagacggttcggcttcccattaccgtctctttccttgagtctggtacagcgcgagtcacggtggacgaagagaagcgccagaaaggagagattgagttgcgccatggcagcaaagcgaggaaagagcgctataacgaggcagagaaatgggccattgttggagggatgactttggacaaggaggccaaggtcgattatgaggacaagacgcagattaccgtcaagtacgggccaacctccaagttcgaggcaaccatcaaattctctcccttcagcatcgacttcaagcgggacggcatcagccacgtcaaactcaacgaccagggcctccttaacatcgagcactggaggccaaaggttgacaaaccggagcccgagaagaaggacggcgattcgactgaagagaataaggcagaggaagcaaaggaggagcccaagggagaggatgagtctacctggtgggaggagacatttggcggcaacaccgataccaagcctcgcggcccggagagcgtcggtcttgacatctccttcgtcggctacgaacacgtctacggcatcccctcccatgccagctcgctgtcgctgaagcagacccgtggcggcgagggcaactaccaggagccgtacaggatgtacaatgctgacgttttcgagtacatcctcgatagcccaatgactctctacggctctattcctttcatgcaggcccatcgtaaggactcgagcgtgggcgttttctggctgaatgccgcggagacgtgggttgacatcaccaaggcgaaagactccaagaacccgctctctctcggcggcaaggccaggaccaacacgcatacccactggttctccgagagcggtctgctggacgtttttgtcttcctcggcccgacgcccaaggatttgaccgccaggtacggtgagctcactggcaccactgcaatgccccaggagttcgctctcggataccatcagtgccgttggaactacgtctcggacgaggacgtaagggatgtggaccgcaagatggacaagttcaagatgccgtacgacgtcatttggctcgacatcgagtacactgacgagaagaagtactttacctgggacaaacactcgtttacagaccccatcggcatgggcaagcagctggacagtcacggcaggaagctggttaccatcattgatccgcacatcaagaataccgacaattaccccgtcgtggcagagctaaagtccaaggaactcggcgtaaagaacaaagatggcaacctcttcgagggctggtgctggccaggctcgtctcactggatcgacgccttcagtccggctgcgcgcgagtggtggtcgtcgcttttcaagtatgacaaattcaagggcaccatggaaaacacgtggatctggaacgacatgaacgagccgtcggtctttaacggtcccgaaaccacgatgcccaaggacaacctccacgacggaaactgggaacaccgcgacgtgcacaacctgaatggcctgacgttccacaacgcgacctaccatgcgctcctcacccggaagcccggcgagctccgccgcccgttcgtgctgacccggtccttctttgccggctcgcagcgggtcggcgccatgtggaccggcgacaaccaggctgcctgggaccacctcaaggcatcgattcccatggtcctcagccagggtatttcgggcttccccttctcgggcgccgacgtcggcggcttctttggcaacccggagaaggagcttttgacgcggtggtaccaagccggcgccttctaccccttcttccgggggcacgcgcacattgatgcgcgacgccgcgagccatacctcgccggcgagccgtacacgaccatcatcgccgcggcgcttcggctgcgctacagcctgctgccgtcgtggtacactgcgttccggcacgcccatctggacggtacgcccattatcaagcccatgttctacacgcatccgtccgaggaggccggcctcgctatcgaggaccagttcttcgtcggcaacacgggcctgctcgccaagcccgtcacggagaaggagaagacgacggtcgatgtgtggatccccgacggcgaggtctactatgactactttacgtaccaggtcatcccgacagtcaagggcaagacggtcacgctggatgcgccgatggagaagatcccgctgctgatgcgcggcgggcacgtgtttgcgcgccgcgacgtgccgcggcgctcgagcgcgctcatgcgctgggacgactacacgctcgtcgtgacggtgccgcgggaaaacaaggtcgccgagggcgacctgtacgtcgacgacggagactcgtttgagtaccagaacggccagtacatccaccgccggttcgtctacgacggcgcggccaagagcctcagctcggtggatgccgagggccgcgacgcggcctcgatccgggagggcgcgtggatgaagcagatgcgctccgtgggcgtgggcaagatcgtggtggtcggcgcgccggccagctgggcgggcaagaagagcgtcaaggtagagagcgagggcaaggtctgggaggccaggatggagtttacgcctgctgggcagggcagagccgcgttcgcggtgatcaagaaggtgggcgtgaggatcggggcggattggaaggtcgagtttcacgacgagctctgaggccctatgacctcccgagtcctggtcgagagaggggagctggagagagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtatttaaatcactacacactcgagccgatgttttacgaagtcccccatggcgtgttctggcggtgtctcccttttggaatgaaggctttttttttggttttgctgatcttgatggatgcttttcaggggcatgcccgcctgggccaccgggtgatcccgcgactggaggggatgaccgccacgtgagattgagtcccgtctctcccggcaaagagcggtttagttccgtgggcaacacatgcatcacacgcgtgtgcacaaatctctgctttttccgacttttggccggtgtgaagaaggatcgacgcaaaaccgggtacattacgcactaagctagccaggccgatacggaagtttccagcacgagcgacccccggagcctgagctccccgaagcaggcaatcagatgtccccccgttctcagcacggagaagggcatgacgtcgactcgtgcggacaaggaggcctgttcaggaggcgcaatacactacactaggcacagcacagcagcagcaccttgataggcacgtccccgtccccatcatcgtttggtacgtgaagtggataaaaattggaatgtgtggcgtgagacattgctagagctagtacggctttacatagtaatgccgagatcacgcattaggtcctcagtgggcagagatggcggcgaatgtcggcaatagttatccctctttcgacgctgcgatagggcagaaaggcattcggattttcaggtttctttttctttttcttttttttccttaaatttcttttcttgtctccgttttcggttttgacatcgctgtcagctgcataactaatgcaagggcgcactagtgtaccgcgtccactagcgttctgcggagtagaagcgctgtgtgcgtgtgtgtgtgtgtgcgtgtgcgcgtgcacgcctctgggaaaagcgccgagccgttgccgtgtggagcacctggttcctgcatccgttctgtcgatcatgataggaatatggcaaacatccctacacactagtgatcgttgtcgtgtttgtttggcccgactgaacggttggcggtatgcgcacctcaggttgaactattggtggtgctttgacccttgaccaacaaactggagccccagcattacggagtagccaggggtgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 19 - DNA, 15270 bp, synthetic construct
  source 1..15270: mol_type = other DNA
tttaaacgaagcgaccaaggtcttccaactgcgttgctactggtggctggaagaccagtatccttcaaagacttcaagtgccggccaacctgggtctttctagtcttgaatagctgagccgcgtgccgaatactgggttgctttctatggggtaaacggctggttcggtggcgagcttgagcggcaaggagccatttggcggcccgtatactagccggttcctggatttggtcagtcagaggcatcgtatacaggagagtaggttcaactgggcttgtggcagtggttgtgtcttcgagtatagggagttggctggtaaaattggacacggatttggacgcatacctgtccggctatattggcatgtccgggggttttggccccacctgtcgggagacaggtggtagtcctgaactaattagtcagcaactaaccaccgggtacttcttctgcccaagacccgattgacgggcgcgaccccaacgtggaagcgggtcccaagttcagcatccctgcagccctgaaaatcgcgccggcgccggggagtacgcggttgtaaccttgttccccggcccaatcatttagctacaaactagttactccgtagctggtcaaattccggaccgaggtgcggacggagcccagaccgtgctcgaacgcggattgttaccccacattccgaccacttaccatagtaggcaattgttggcccaggagctgcaagaccatgacataacggaatgcacctatttttgttgtctttgaggagtgctttcttttccacgtagttaggttcttttctgtccgcttagagcggcattggcattgctgtctctatatccgttgctttcggtaccacaggaggtgttcggctttcggcacaggaagtggagaggatggtttacgaacagaaccaactgcatagtaggtgcctacccaatcacacaacccccccccccctgctgctataatgtatacgagaaggccgacaatagaaccaacatctccgcacacccggtgacgggaatatcgggccctgctgattcccatggtcgagaccccccgggctgacagccagccgcaggcaccctctcatcgtgccgggccggtctccggacactgacagtagactgaatacagccacaaccaactcacagcccgggcggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatggcggttgctcgccccgtgagggcgctggcgctgacgggcgttgtcctgtggtgtttctttgtatggcagcttttcaagccaacaggatcgcccaagagcccggccagggtccctctcctgtcgagagagcgcgatcctaatctagaccgtaggcactgaacagcccagggcggccttggaaaccatttgctgacttgaaatagccaccggcgagcccgagggtgttctgacatatgcgtccggggagtacgcacccggcccctccgtcagcgccctcgacggcgaccccgcctccctgacccgcgaggtcatccgcctcgcccaggacgccgaggtcgagctcgagcgccagcgcggcctgctccagcagatcggcgacgccctctcctcgcagcgcggccgcgtccccaccgccgcccctcccgcccagccgcgcgtccccgtcaccccggcccccgccgtcatccccatcctggtcatcgcctgcgaccgcagcaccgtccgcaggtgcctggacaagctcctgcactaccgcccctcggccgagctcttccccatcatcgtctcgcaggactgcggccacgaggagaccgcccaggccatcgcctcctacggctcggccgtcacccacatccgccagcccgacctctcctccatcgccgtcccgcccgaccaccgcaagttccagggctactacaagatcgcccgccactaccgctgggccctgggccaggtcttccgccagttccgcttccccgccgccgtcgtcgtcgaggacgacctggaggtcgcccccgacttcttcgagtacttccgcgccacctaccccctgctcaaggccgaccccagcctgtggtgcgtctccgcctggaacgacaacggcaaggagcagatggtcgacgcctcccgccccgagctgctctaccgcaccgacttcttcccgggcctgggctggctgctcctggccgagctgtgggccgagctggagcccaagtggcccaaggccttctgggacgactggatgcgcaggcccgagcagcgccagggccgcgcctgcatccgccccgagatctcgcgcaccatgaccttcggccgcaagggcgtctcccacggccagttcttcgaccagcacctcaagttcatcaagctgaaccagcagttcgtccacttcacccagctcgacctgtcctacctgcagcgcgaggcctacgaccgcgacttcctggcccgcgtctacggcgccccgcagctccaggtcgagaaggtccgcaccaacgaccgcaaggagctgggcgaggtccgcgtccagtacaccggccgcgactccttcaaggccttcgccaaggccctcggcgtcatggacgacctgaagtcgggcgtcccccgcgccggctaccgcggcatcgtcaccttccagttccgcggccgcagggtccacctggcccctccgcccacctgggagggctacgaccccagctggaactaaggcaggcaggagttggagtatgagggtagccgctgatggctattcttcccacgtttttgtgtgtttcctcttcatttttttttctcttgccgcaacatgacggctcctgtctctgaagggaacccctgaaattcagggttatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtccgcgatgagcagagcattttaaaggaacgccgcactcacaaacaccaacactttagtgtctagtctacagaggcgtccctccccgtcttggatgcgtgattccattaccgtagatagtaccgcaaatgcacgggggtgtagtgtatgaaccacgctgggttcctgacctgacccggcaacccaatggagcagactcagggcccgctggccccggtggcgtatcaggtgactgttgggggagctaaccttggcaaacaaccgagctcagcgttaatgcatttcaagaagtcggtttgattgatcatccgcgaggaccgattatcgtacggcatcgaaaatcgtctcgccggagcgcacggattatttgaagaggctggcttgttgattgcaattgtcggctgccggccacgtcaccggccttgcagggcttatcagtaaatgcgggggcggagcagaggcggttcttgtcaagggtaggaggggtccggcaaagcccgagacggtggctgttcggaaacccaagaatggaccctgacagaacaattttcggattgggttcgttgcaaggatcgaacactacatcttccgagagagtttggaggttgtaagaacccttcgctaccgggagaacaaatcaccttgttgaatcagctctgtcactgctagtggcgagatggcctaagcagcgagactgttccccctgccccgctgtggatccgcatgactggtccattctggtcacttcgctccacttctctgcttttgcattgaccgctcagcggctgttgcgccttcctgacgcattcatagccccactcctgggcggcagcctggcgcttccaccatgcttgcccaacacgtatataaccttctcggcctaccctctaccacggagccactttctcttctccaacatcctccacacaacacccttctccttcgccatcaaagaggcatctatcggaaaatccaacatcgccagactcaccgaaacttcatacactcataacaactgcaaccatggcggcgacagagaaaacgggcagcagcagcacgcgagcgctgcgaggcgcgtccctgctgatcgtgctgcaagtggtctcgcgcgccatcacttttgtggccaaccaggtgctgctgcgatttctgactgcccagcttctgggggtgtcgacgcagctcgaagtctactacctgtccgtcatcttctttgcccgggagagcctgagggttgccatccagagacaggacctgacaggcctcccggacaagaaggacaacaaacagaatggccatgtcaaccaatctgcccaggccgtagtcaacctgggctatctcgcccttgccctgggcgttccgctcgcatttctctttgggtggctctatctcgactctctgagcgcaagcaccctgctatccgcgcccaacttggtcgtttccctctacatctacgccgtcgccgctgtcctggagctcgcctcggaacccgccttcgtcgtgatgcagactcgactgcagttcggcactcgtgcagctggcgaatcggttgccacgtttctgcgctgcatagtcacgctcggaacagcagcctggggagccggtcgcgatctgggcgtcctgccgtttgccctgggccagttgagctacgggcttgggctgctcgcagtctacacctggcacggggccgcgctggcaaggagggaaggcttctcccttcttcctctccgcatcaccgctccttatagcacgacagcaccacctcaacaaccatcgggcaaatccgtcttcgtcctctcctacttctaccgccctaccctgcagctcgcctccagcatgatggcccagagcgtggtgaagcacatactcacccagggtgacacgttcctcgtcagcatcttgtccacgcccacggcccagggggtgtatgccctcgccaacaactacggcggccttgtcgcccgcctggtcttccagcccatcgaggaaagcagtcgcagctacttcagccgccttctcgccccttccgatgctgctgctgccgctgctgccgctgccgccgccgccgccacagaagcgtccccagcaagacagtcttctccggaagccagatcgaaaccccaatccgagaccgagaccgagccagcagcggaagcagcggaagcagcagcagcagcacggggggcagtctctcgggccaaaacagctctccagtccctcctcaaatcctacctcctcctctccctcgtcgtcacggccctcggcccgaccgccgccggccccctgctctccctcgtcgccggccgccgctgggccggctccggcgcgggcgcctgcctggccgcctacgcgtggtacgtcccgctgctggccatcaacggcgtggccgaggcctttgtggcctcggtcgcgaccgaggccgaggtccaccggcagagcgcatggatggccgccttctcagtcgccttcgccgccgccggcttcgtcttcctgcggctactgggctggggcgccgtggggctggtcgttgccaacggcatcaacatggcctgccgcatcgcctggtgtgccgttttcataagccgatatttcgccgcgaaggggcaccgcttcgacctgctcgacgtcatgccctcgcccgcgtccgtgctggcgggcgcggttgcctctcaggtcgtccggaagctcgttcgctcggcagcatcagaggaggaggtcccacttggcgtgaaggaggccgtgatggacctggtcagagtcgcctcggttgccctgccgtttgttcttgtcgtgtaagtttttcttttcttcgtgttttcttttccggaccctcttacgatgagacgttgaatgctaacaacgcgaaacagtgccgtgtccgaacggcggttcttgtacggcgtgttccaggctcttcggggaacccgaggggctccatgacgcgcgcgagtgtctgcatcggacgggaatgggcctgggagcgttttagcgggtttgggacggccaaccattggctgccgctggaaatttggggtttaccattaatgacacggtaacatggagataccacggatgaatagactcgtttggagtcccccgattattgttcgtttgatgctgcgtaatcgtggtgcgatgacatttgatgcctatgggatggcgggggtctcccccgctttcggaagttgcatgtgaaaaacagttcctgctccgtcctagccttggcaatgcaaacttggatgttccggcttcgtaaccgcctttcacatccttcctccgacaatgcaggttgttgccgacaagccagcacgtcaatgatcctcatgatgcagcttgctgcaagagagcgcaagcttcgagaagcagagcattcattacctcccgtgcctccgtgaacacgtctcgtctcgtcggtcaaagttttgccaccatcatcctacactcatttaaatggcgcgccgttggcggtaacagcaggtgccgttcgctgctgctcggtccttttcccgaaagtaccctgccttcctggcctcgacctccgcctgcggggctctgggcactgtcaaactttcgcagttttgcacccgcagcgattcgagcgggacccgcttcgccaacgcggatcaggagaggtaaaagtctcccctgtcctttctgccggagcatcctccgtccgtcgttcgctggttgttgtgtgtgtgtgtgtgtacagtactgtaggtacatagtgctcaccccagggcatttccccaatccgcgattgcgcataccataggcgtcatctgaatctgcgttacgtccggataacactacgcagtacgaaggagtcggtgctactacgcccgcaagttcaagtctacggtgtggctaaggtgctcaagggcatagttacacgcaccccgggcgagcttctcgcacctcgaccctttgccggggcgtcactggtccgacaactcataatccaccatcgcttgggcaggcctgcatcccccgtgggtcgcatccaagccgccggtaaataccggccgacacacacacacacacacgcaccctgtctagcgcgacgctacaaaagagaccggcaggcactccccgcgaccgcttgttctggccatcccgtctctcgcctctgcagttatcgcctgcctcatctctgcgtcctgataacttttgtcacctctgatccccccccgacgagacggtctcgccgaccaggacccgagatggcggaccgccatcctactctgtcccagtccttcgccgagcgggcgaagacggctagccacccgctcacccgctacctctttcggctcatggacctcaaggcctcgaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 20 - DNA, 15281 bp, synthetic construct
  source 1..15281: mol_type = other DNA
tttaaacgaagcgaccaaggtcttccaactgcgttgctactggtggctggaagaccagtatccttcaaagacttcaagtgccggccaacctgggtctttctagtcttgaatagctgagccgcgtgccgaatactgggttgctttctatggggtaaacggctggttcggtggcgagcttgagcggcaaggagccatttggcggcccgtatactagccggttcctggatttggtcagtcagaggcatcgtatacaggagagtaggttcaactgggcttgtggcagtggttgtgtcttcgagtatagggagttggctggtaaaattggacacggatttggacgcatacctgtccggctatattggcatgtccgggggttttggccccacctgtcgggagacaggtggtagtcctgaactaattagtcagcaactaaccaccgggtacttcttctgcccaagacccgattgacgggcgcgaccccaacgtggaagcgggtcccaagttcagcatccctgcagccctgaaaatcgcgccggcgccggggagtacgcggttgtaaccttgttccccggcccaatcatttagctacaaactagttactccgtagctggtcaaattccggaccgaggtgcggacggagcccagaccgtgctcgaacgcggattgttaccccacattccgaccacttaccatagtaggcaattgttggcccaggagctgcaagaccatgacataacggaatgcacctatttttgttgtctttgaggagtgctttcttttccacgtagttaggttcttttctgtccgcttagagcggcattggcattgctgtctctatatccgttgctttcggtaccacaggaggtgttcggctttcggcacaggaagtggagaggatggtttacgaacagaaccaactgcatagtaggtgcctacccaatcacacaacccccccccccctgctgctataatgtatacgagaaggccgacaatagaaccaacatctccgcacacccggtgacgggaatatcgggccctgctgattcccatggtcgagaccccccgggctgacagccagccgcaggcaccctctcatcgtgccgggccggtctccggacactgacagtagactgaatacagccacaaccaactcacagcccgggcggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatggcggttgctcgccccgtgagggcgctggcgctgacgggcgttgtcctgtggtgtttctttgtatggcagcttttcaagccaacaggatcgcccaagagcccggccagggtccctctcctgtcgagagagcgcgatcctaatctagaccgtaggcactgaacagcccagggcggccttggaaaccatttgctgacttgaaatagccaccggcgagcccgagggtgttctgacatatgcgtccggggagtacgcacccggcccgtccgacagcgccctggacgacgaccccgccagcctcacccgcgaggtcatccgcctggcccaggacgcggaggtcgagctggagcgccagcgcggcctgctccagcagatccgcgagcaccacgcccgctggtcgcagcgctggcgcgtccccaccgtcgcccctcccgtcccgccccgcgtccccgtcaccaccccgcccgccgtcatcccgatcctggtcatcgcctgcgaccgctccaccgtccgcaggtgcctggacaagctcctgaactaccgccccagcgccgagcacttcccgatcatcgtctcccaggactgcggccacgaggagaccgcccaggtcatcgcctcctacggctcggccgtcatgcacatccgccagccggacctcagcaccatcgccgtcccgcccgaccaccgcaagttccagggctactacaagatcgcccgccactaccgctgggccctgggccaggtcttccacgagttcaagttcccggccgccgtcgtcgtcgaggacgacctggaggtcgccccggacttcttcgagtacttccaggccacctaccccctcctgcgcgccgacccgtcgctctggtgcgtctccgcctggaacgacaacggcaaggagcagatggtcgacagctcgaagcccgagctcctgtaccgcaccgacttcttccccggcctgggctggctcctgctggccgagctctgggccgagctggagcccaagtggcccaaggccttctgggacgactggatgcgcaggccggagcagcgccagggcagggcctgcgtccgcccggagatctcgcgcaccatgaccttcggccgcaagggcgtcagccacggccagttcttcgaccagcacctgaagttcatcaagctcaaccagcacttcgtccccttcacccagctggacctctcgtacctccgccaggagacctacgaccgcgacttcctggcccgcgtctacggcgccccgctgctccaggtcgagaaggtccgcacctcggagcgctccgagctccaggaagtccgcgtccagtacacctcgcgcgacagcttcaaggccttcgccaaggccctgggcgtcatggacgacctcaagtccggcgtccccagggccggctaccgcggcatcgtctccttcctctaccgcggcaggcgcgtccacctggccccgccccagacctgggacggctacgacccgagctggaactaaggcaggcaggagttggagtatgagggtagccgctgatggctattcttcccacgtttttgtgtgtttcctcttcatttttttttctcttgccgcaacatgacggctcctgtctctgaagggaacccctgaaattcagggttatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtcgcgatcgcgatgagcagagcattttaaaggaacgccgcactcacaaacaccaacactttagtgtctagtctacagaggcgtccctccccgtcttggatgcgtgattccattaccgtagatagtaccgcaaatgcacgggggtgtagtgtatgaaccacgctgggttcctgacctgacccggcaacccaatggagcagactcagggcccgctggccccggtggcgtatcaggtgactgttgggggagctaaccttggcaaacaaccgagctcagcgttaatgcatttcaagaagtcggtttgattgatcatccgcgaggaccgattatcgtacggcatcgaaaatcgtctcgccggagcgcacggattatttgaagaggctggcttgttgattgcaattgtcggctgccggccacgtcaccggccttgcagggcttatcagtaaatgcgggggcggagcagaggcggttcttgtcaagggtaggaggggtccggcaaagcccgagacggtggctgttcggaaacccaagaatggaccctgacagaacaattttcggattgggttcgttgcaaggatcgaacactacatcttccgagagagtttggaggttgtaagaacccttcgctaccgggagaacaaatcaccttgttgaatcagctctgtcactgctagtggcgagatggcctaagcagcgagactgttccccctgccccgctgtggatccgcatgactggtccattctggtcacttcgctccacttctctgcttttgcattgaccgctcagcggctgttgcgccttcctgacgcattcatagccccactcctgggcggcagcctggcgcttccaccatgcttgcccaacacgtatataaccttctcggcctaccctctaccacggagccactttctcttctccaacatcctccacacaacacccttctccttcgccatcaaagaggcatctatcggaaaatccaacatcgccagactcaccgaaacttcatacactcataacaactgcaaccatggcggcgacagagaaaacgggcagcagcagcacgcgagcgctgcgaggcgcgtccctgctgatcgtgctgcaagtggtctcgcgcgccatcacttttgtggccaaccaggtgctgctgcgatttctgactgcccagcttctgggggtgtcgacgcagctcgaagtctactacctgtccgtcatcttctttgcccgggagagcctgagggttgccatccagagacaggacctgacaggcctcccggacaagaaggacaacaaacagaatggccatgtcaaccaatctgcccaggccgtagtcaacctgggctatctcgcccttgccctgggcgttccgctcgcatttctctttgggtggctctatctcgactctctgagcgcaagcaccctgctatccgcgcccaacttggtcgtttccctctacatctacgccgtcgccgctgtcctggagctcgcctcggaacccgccttcgtcgtgatgcagactcgactgcagttcggcactcgtgcagctggcgaatcggttgccacgtttctgcgctgcatagtcacgctcggaacagcagcctggggagccggtcgcgatctgggcgtcctgccgtttgccctgggccagttgagctacgggcttgggctgctcgcagtctacacctggcacggggccgcgctggcaaggagggaaggcttctcccttcttcctctccgcatcaccgctccttatagcacgacagcaccacctcaacaaccatcgggcaaatccgtcttcgtcctctcctacttctaccgccctaccctgcagctcgcctccagcatgatggcccagagcgtggtgaagcacatactcacccagggtgacacgttcctcgtcagcatcttgtccacgcccacggcccagggggtgtatgccctcgccaacaactacggcggccttgtcgcccgcctggtcttccagcccatcgaggaaagcagtcgcagctacttcagccgccttctcgccccttccgatgctgctgctgccgctgctgccgctgccgccgccgccgccacagaagcgtccccagcaagacagtcttctccggaagccagatcgaaaccccaatccgagaccgagaccgagccagcagcggaagcagcggaagcagcagcagcagcacggggggcagtctctcgggccaaaacagctctccagtccctcctcaaatcctacctcctcctctccctcgtcgtcacggccctcggcccgaccgccgccggccccctgctctccctcgtcgccggccgccgctgggccggctccggcgcgggcgcctgcctggccgcctacgcgtggtacgtcccgctgctggccatcaacggcgtggccgaggcctttgtggcctcggtcgcgaccgaggccgaggtccaccggcagagcgcatggatggccgccttctcagtcgccttcgccgccgccggcttcgtcttcctgcggctactgggctggggcgccgtggggctggtcgttgccaacggcatcaacatggcctgccgcatcgcctggtgtgccgttttcataagccgatatttcgccgcgaaggggcaccgcttcgacctgctcgacgtcatgccctcgcccgcgtccgtgctggcgggcgcggttgcctctcaggtcgtccggaagctcgttcgctcggcagcatcagaggaggaggtcccacttggcgtgaaggaggccgtgatggacctggtcagagtcgcctcggttgccctgccgtttgttcttgtcgtgtaagtttttcttttcttcgtgttttcttttccggaccctcttacgatgagacgttgaatgctaacaacgcgaaacagtgccgtgtccgaacggcggttcttgtacggcgtgttccaggctcttcggggaacccgaggggctccatgacgcgcgcgagtgtctgcatcggacgggaatgggcctgggagcgttttagcgggtttgggacggccaaccattggctgccgctggaaatttggggtttaccattaatgacacggtaacatggagataccacggatgaatagactcgtttggagtcccccgattattgttcgtttgatgctgcgtaatcgtggtgcgatgacatttgatgcctatgggatggcgggggtctcccccgctttcggaagttgcatgtgaaaaacagttcctgctccgtcctagccttggcaatgcaaacttggatgttccggcttcgtaaccgcctttcacatccttcctccgacaatgcaggttgttgccgacaagccagcacgtcaatgatcctcatgatgcagcttgctgcaagagagcgcaagcttcgagaagcagagcattcattacctcccgtgcctccgtgaacacgtctcgtctcgtcggtcaaagttttgccaccatcatcctacactcatttaaatggcgcgccgttggcggtaacagcaggtgccgttcgctgctgctcggtccttttcccgaaagtaccctgccttcctggcctcgacctccgcctgcggggctctgggcactgtcaaactttcgcagttttgcacccgcagcgattcgagcgggacccgcttcgccaacgcggatcaggagaggtaaaagtctcccctgtcctttctgccggagcatcctccgtccgtcgttcgctggttgttgtgtgtgtgtgtgtgtacagtactgtaggtacatagtgctcaccccagggcatttccccaatccgcgattgcgcataccataggcgtcatctgaatctgcgttacgtccggataacactacgcagtacgaaggagtcggtgctactacgcccgcaagttcaagtctacggtgtggctaaggtgctcaagggcatagttacacgcaccccgggcgagcttctcgcacctcgaccctttgccggggcgtcactggtccgacaactcataatccaccatcgcttgggcaggcctgcatcccccgtgggtcgcatccaagccgccggtaaataccggccgacacacacacacacacacgcaccctgtctagcgcgacgctacaaaagagaccggcaggcactccccgcgaccgcttgttctggccatcccgtctctcgcctctgcagttatcgcctgcctcatctctgcgtcctgataacttttgtcacctctgatccccccccgacgagacggtctcgccgaccaggacccgagatggcggaccgccatcctactctgtcccagtccttcgccgagcgggcgaagacggctagccacccgctcacccgctacctctttcggctcatggacctcaaggcctcgaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 21 - DNA, 18195 bp, synthetic construct
  source 1..18195: mol_type = other DNA
ggcgcgccgtttaaacgaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagggcgacggcctcggccagcagtacaactcgcccagcaagttgatcaacatttgcggcaccgacattgtcatcgtagggcgtggcatcaccgccgccggcgacccgccctccgaggctgagaggtacaggagaaaagcctggaaggcctatctggcgcgtctggcgtgatttggggggagggggagaggagatgggggacgggaggggtcgccttggtcagtcttgtgcgtgtcctgcagcggattcgtcaccggggcagcacccaaaagagggagaaaaaggggaaaaaaaataaataaataaaaagggttaagttgttgaaaaaagtgttgtgagctctctggcaaggcgcgccatttaaatcgttggcggtaacagcaggtgccgttcgctgctgctcggtccttttcccgaaagtaccctgccttcctggcctcgacctccgcctgcggggctctgggcactgtcaaactttcgcagttttgcacccgcagcgattcgagcgggacccgcttcgccaacgcggatcaggagaggtaaaagtctcccctgtcctttctgccggagcatcctccgtccgtcgttcgctggttgttgtgtgtgtgtgtgtgtacagtactgtaggtacatagtgctcaccccagggcatttccccaatccgcgattgcgcataccataggcgtcatctgaatctgcgttacgtccggataacactacgcagtacgaaggagtcggtgctactacgcccgcaagttcaagtctacggtgtggctaaggtgctcaagggcatagttacacgcaccccgggcgagcttctcgcacctcgaccctttgccggggcgtcactggtccgacaactcatttaaatcatttccacaactcatgccgagagaagtgtcagactgggcaactaaagtagtagtagtaatagctcgattaccatgatgaaatgctgggcgtcgaagcagctgcaggtccggcatgcagcaatccccactccgctccatcggctgctgttctggggtcaatccgtaccctcccaagttcacctcgccgctgacctcgggatcagctgcgttgtgcattcatgaataatgcgcaacatgagcaacccaacttcatcaagggagtttcgacgtcaccatatcaccacacatcttggaacagaattggggacaaggcagctggattgacggggaaatacataaaccggacgacctatgacgaccatcctatcccgtcaccgactccgatcccctgcggatgatggctcaaagaaccaagtttgatgagccggctgtgtgtcccagcacatacaacaaccgaagtatcagccccgttccgaacgcaggatcccagtctaccgaatcgattttggacagcccgagagaagccaaacaccgaaccgaaggaggaattgtcggaagacgtgcattatgagtcgttcacgcgcgcgttagcggcggttctccgactcgcgcatccggcgcatgtactcctcctggtagctgccgacgttgttcttgcggtcggcgttggtgacctggtcgaaggggacgcggtgggccagcatctcctggatctccttggcgggcgggtactggcccgggcagatccaggagccgggcgggtggcagacgcggttggccgggtcggcgacccacttcttgctctcggcggagacgttcatgaccttgaagatgcggaccaggccgtacttggaggagtagacttcctggaagagcgaggggttgaccttgacgcccttgcgcttgccggcctcgtgcaggttgtacaggagggaggcgcgcatcatcggggtggggcggctgtagtcgttgcggtggaagccgaactgctggcacagcgggtcgtccgggcagatgtcgtggtagacggagttgccgatgcgggccatgtgcgggctcttcatcaggtcgcccgactggccggcccagatgaggacgtagtcggccatgtggcggaccaggctgtgggcctcgacgaccgggctggtgagcatcttgccgatggtggcgatgtgctcgtggttccaggtgttgccgtcggccagcgaggtgcggttgccgatgccggtgatctggtagccgtagtcccaccaggcgaggacgcgggcgtcctccggggtgctgtcgcgcagccactcgtaggccttcaggtagtcgtcgacgagcaggttcatcggcttgccggtggcgcggttctggacgacggcggcgaagacgatcatggggttgctggactgctcggcgaacttggtcgagtggctggcgaactccgagctgaagaagctgacggcggtggtggtgaccagggcccacatggcgatggacaggaccatgcggtggccccaggccagggaggagccggcgaagacgtcgcagaaggcgcgggcggtggtggcgttcttggcgtcgtcgcggcccgagcccttgccggcgcccctctggtggcgctgggcctgcttctgctgcttcttggccttggtggcgtcgctgtcccagaaggagagctggacggcggcctccaggatggtgccgacgaagatgccggtcgacaggcaggcggcggggcccgacaggagcaggaggcgggccatgcgggtggagaagtagtagacggcgcccgagttcaggagccagaagaccttggagggcgagtagtggacgaaggtcgagacggccaggacgatgctgcccaggccccaggtgacgccgcagacgtggaggaaggcccacatggcctcgggcgaggccggctggtgctcggcgacggagtcgacgagcgggttgccggtgcgggtgtgctcgacgaagagggcgcggacgcggacgctcagggggccgaagtagccggtgggggcgaggaccgagatggccagggcggcgacgccggccatgacgctgaagacgcggacgcggatcttgaagttggcgcgggagcggacctcgacgccggcgcgggcgcggaggacctcgcagacctggaggccgcacaggaagacgaggaccaggagggcgcccagctgctccagggacttgaacgggctcatgccgacgggcgggacgcagacggcgatggcggtgccgacgacgtagaagagggtgtaggcgcgcaggaggctggggttgtaggtgttgcgggcccagtcgaccatgctggagatgccggcgtgcatggcgaccatgttcaggacgaagatgtagccgccccaggcggcggccatgtagccgtaggcgacgccggtcaggacgccgatcggccaggagctgcgggtgcggagggagcggacccagcagtagaaggtcaggagcatggcggcgacggcgatgcactcgttgtcgaactcgccggccatggagcgcatgaggtgggccgggatgatgctgaaggacagggcggcggcggcggcggcgacggtggagcccgaggcctcgtaggtgcagaaggcgagggtggcggtggcgatggcgccgaaccaggcgggcatcaggacgcagacgttgttcagcgacatgggcatgccggcggcggccagggcgcggtggatggcgacggcggtcagctggaggcccgggtaggtggtgctgccgacgggcctgccgagcgggtaccaggacatgtagtcgaaccaggagaagaaggccgaccagccgtgggtgctcatgtactcggcggcgcggtagttgaaccaggggtcgaactcgtggatgaggtagccgtagatctggacggagatcatgcggacggtgaaggcctggaagcaggaggcggccaggacgaagagggcgacgacggtcaggacgaagtggaccggccagaacgggaaggggaagatgccgatgaagtccttctcgtcggtcagggtcttgggcaggaggatgaccttggcgggcgggctggcggtcttggtctgggaggcggcgtcctcggcctgggcggaggcctcgcggctggcggtggcggcggagccggagtcgcccagggagttgcccttgcgcttgcccatgttgcttgcgtggtgtgctggaagctgagtgtattaggtggattgacaagtccctgcgggcaacgggaccgagtgagcaagccaggatcaggcgagcaagaggcaggtggtctgattctatcaacctacgtttagagacttgagatggaccagggaatgggcgttttgttttcgaattgatggtttacgatggatttcgttggacggaagaccgatgaggggaaaggagaggagaagcccaaagagggggtcggaggtggcctttattaagaggcggccggccgggcaatgggcagatcagccatctttgctgcatcgttcctgcgactgtttcgtcagaccgggcggggtaatgtcaggagagctccctggtaggttgcgggcagcggcagtgatcacgttgactggctcacgggatcgcgtgacgagtacatcatgatggcacgacctcgcaggcgagccctgcgtggcttaaacaggccaaggtacctggcccgagcgtcctgcagccagcgctaacagcccagcccaagccagaatctggggtaatctggggtaccggggtgcccgacccactgcgggcaaccagcgcttgtgcaccgcgtaaggcctcaacaagacatcagttagtatcgatgccgagattcagttggcaattacatacgtctaacttttccaatgcttattttgagtttcttgtagttatgcagctggtggaagttaggacaggaaccttgagtgacaagcaatccggccgggccgggaaggtgcccgctgcacgatcagtggggcaaaggtggggtatcccgagcaggagcgaactccaacagagtattcgatcaaaaaaggcaagtcctcccccaccatcctttgtagcttgcaatgcatctccttttttgcaatggattttgcttcgcgagtgctaatgccttgtgaaggactatgtggttggttcaaacctgttgttttgatccatctagtccacgttgcaggcatacaaatacccgacgaattcgacgtgtatcataagttaagtaggtactgtacgttagtttgtttaccttcttcagccagtagtccgactttgctctcaagtgctcatccaaccctttggccttccaaatcttgataccgagagcctcaggcaggttggtatattctacctcaaatacctgccagatgttgaccaatgccgttgtcgttactccgtactacgcgaaccaatgccaacatgattctcctttcagtcacgagacgattcgagtgtcatggtagcagtactttggtagtaaagagtcactcaattgaacatgtcgtagctcattcgtaaccaagtcatgataaccctgaatttcaggggttcccttcagagacaggagccgtcatgttgcggcaagagaaaaaaaaatgaagaggaaacacacaaaaacgtgggaagaatagccatcagcggctaccctcatactccaactcctgcctgccttagctgggggtgccgatgtcgacggtgatctgggtgtagagggggtagcgctggacgtcgaggacctggtaggtgaggctgttgaggccgtcgctgagcatggtctccttggtgtgggcgattcggtcgaagcgctgggggttgggctcgttcttcttgtcgcggctgtggcggatcatgcggcagcggccgacgacggcgttggggcggctgatgctcatgccgcgaaagacgaggcggttaaagatgtcgtcgtcctcgccgccccagccccagtagttgttggggaagccgttgatggtgaggaactgctgcttgctgagggcgctgacgccgccgaagtactggacgtaggggaggctgaagccgaacttgtccatggcgacgctgatgtggcggggctggctgaagcagcggtaggcgttgtggtcgttcatggggatgaggtcgacgtcgctaaagacgaagcacgtgtagtcgtagtccttgagggcctcctggaagccgacgttgaggagcttggcgcggttgaagatggtgtcgccggcctggttgatgacgtagatgccgtagtcgagctgctggcgctggaggacggggtggaggtagtagagccagtacttgaggtgctcctggcggttgcggaacgggatgatgatggcgaccttgtgggggctgacgcagtcgcgaggggcgtacctgccgcccatcttgacgttggggttctgcttggcgaccagctcgaggtcgacgggcatgttgaactcgatgagcatggggccgacgaggagggggctctcctcggggcaggcggggaggctgagggcggtggtgtgggggacggggacgctggtgaggttgctggcagggccagggccgctgtcgacgacggggctgctgtcgccgccagggcgaggctggctgctggcgcccagaggaggcggagggcgggcgccgccggttcggggggcgtcggccttctcggcggcggccttgagggccttggactcctcgtccatggcctccgagtcctccgaggcctcggagttcagggcggactgctcgagcttcttggcgtcgttctcctccgagatgacctgctgctccggggagatgctgcccgaggtgaagtcgaaggcggccgagatcgagctcgggatgtactgctgggtgcgcgagttgctgttgagggtgagcaggaggacgatgacggcgccggcgatgacggtgaagcgcaggaggcgcttgctcaggaagagggccatggttgcagttgttatgagtgtatgaagtttcggtgagtctggcgatgttggattttccgatagatgcctctttgatggcgaaggagaagggtgttgtgtggaggatgttggagaagagaaagtggctccgtggtagagggtaggccgagaaggttatatacgtgttgggcaagcatggtggaagcgccaggctgccgcccaggagtggggctatgaatgcgtcaggaaggcgcaacagccgctgagcggtcaatgcaaaagcagagaagtggagcgaagtgaccagaatggaccagtcatgcggatccacagcggggcagggggaacagtctcgctgcttaggccatctcgccactagcagtgacagagctgattcaacaaggtgatttgttctcccggtagcgaagggttcttacaacctccaaactctctcggaagatgtagtgttcgatccttgcaacgaacccaatccgaaaattgttctgtcagggtccattcttgggtttccgaacagccaccgtctcgggctttgccggacccctcctacccttgacaagaaccgcctctgctccgcccccgcatttactgataagccctgcaaggccggtgacgtggccggcagccgacaattgcaatcaacaagccagcctcttcaaataatccgtgcgctccggcgagacgattttcgatgccgtacgataatcggtcctcgcggatgatcaatcaaaccgacttcttgaaatgcattaacgctgagctcggttgtttgccaaggttagctcccccaacagtcacctgatacgccaccggggccagcgggccctgagtctgctccattgggttgccgggtcaggtcaggaacccagcgtggttcatacactacacccccgtgcatttgcggtactatctacggtaatggaatcacgcatccaagacggggagggacgcctctgtagactagacactaaagtgttggtgtttgtgagtgcggcgttcctttaaaatgctctgctcatcgcggccgcgcacacctacttgtatacactcccaagcgtagccccaagagcaggaaaagaaaagtgagaaacaagaagagatttgctgctcagagcaacccccagctgcaccctccccaccactcgcgtctccaatttagtggttgccgtgggctcataggttggggtccctatggatttgggggtggacgctgcaggatcggagtaatcatcgaggaggacgacgctgcttgacaggccaggcgtaaaaagaggcaatcatctgatggtgtggtgtgagagaacgggtcaagtaacagtcgggggaacgttggtctttggtttgggttgaatggtacagttgattaaaacaaaaagacagtgacaaggggggaatattctaccaaacaacgtccgcacgacccgcgaggctagccaagaacgcacgacccaaaccacgtccgcagacagggaaaacgagacaaatcacggacaacaggagagaccagccgatttttgttgttgttgttgttttactgcagcctgcggtaggacttgcagagctcgtggtcgcggatgtcgccccagccaccgttcttgcgcgggggcgagatggcggccatggggaacttctcgccgatgaccagggtctcggggaagaggtactgcttgttgttgttcaggaggctctcgatctgggcggactgggtgctggggcggcaggtcttcttgtggtgcatgccgcagtcgccggcgtggaagatcctgggggcctgcgggaccaggaccttccagaccttggggaggcaggccagggtgaggtactggagggtccagtcccagttgtagtcgtcgtaggtgcagaaggtgtcggtgcactcgatcagcttctggtaggcgtcgcgggtcagggccaggcccatgttgtgctcggtcgacttccaggtcttgacgtcgaccttgtcggcgatgccgtagaaggagcggatggtggtgtaggtgcccagggagaggacgtcgcagccggggcactcctgctgcttgagcttccacatcttcttgaagacgtggtagaagtcgggggccaggtagtggtcttcctccaggaagaggatcaggccggtgtagtcctggaggaccttgacgcgctcccagacgaagtgcagcttccaccaccagtggtgcttggtctgcgagaacttggcctcgcggtagtggccgaagctgtcggggtactcggcgttgatgcagccgagcttcagggcggcgttcttcttgaggtcgcgggggcagtcgcgggggtccgagccggggaactcggaggggtagagctggatgctgaacgggaagaagacctgcaggacggggcagaagtcgacgctggagatgagcgagttgatctcggcgctccagaagtcgtgggagaagatgacgaggacctcgcggatgccctgggccttgcggagcgagtcgatcaggaggcggaggtactccgggcggttgtggacctggacgacgaggaccagctcgcccggggaccaggtgccgtccttgtcgacgttgcggagcatctggtcgaagttcagctggtagacgaggctgcggtagcggagggtcaggttgtcgacctccgggcgggggacggcggggacgagcggggcggccgagtcgttcgagaccctgcggatgccgacgctggcggcgaagtggccggcgcccctcagcggctcggagtccaggaggggcggggcgagggcgtcgttcttgcgctggcggccgttggagctccagaggacgaagccgcaggcggcgacgaccagggtcaggatgaggaccttgcgcttgtagatgcggaagcgcattttgacggtatttgtgttctgaagaacgaaactggcgacttgcgcttaatgaaaactgcaaaaaggaaagttagagacgatgtacttgtcaagaaccagtgtatcgatgatggtatacgcacttgaaagtggcaggtgattgcttgaatcaggtatgccgacgaaggaagtgaagaggaagaatcgaagaagatttgaggaggggtcgaagtttttatgggcaggcgggaggggcaaggcgggttggtggggttacgcagcacaaaaacttcaaccacggtggggcaagccctcgaatttcgccaggaatttcacgtcgtgctttgccgcccagccccgcgattcaggcttcacttcctccacaaccgcatgcaaatcgccagagggctgcatcccggtattttgattcgcacccgacaagtaatgatagggctctaaatacagctagaggagtccgcaacgcgatgatggactctttgaacaattattaagtacagtcttctctttctcttcagaccagcgcaacacacccctccagatttgaaactgcccctctcgcgctcgcacagggaactgaactggcaagcccctctttttgccaatcgtttgagcctccctccctgcccttactgagcattggccgcctctggtgccactacttggtatggaactggcatgtcacgctggcctttaagtgggacgttgcgctgtggcccccggtccgttaggcattggcgctcagcccaaccttacgaagttgttcatggaataatggaggacggatttgcagcctgcggcgtgagtcaactaccctccgatcatgcaaatccgcctcccattttctgcaagtttgaagtccacattctgcagcaacgagatgttgtttttatccatacaacgctcctcctaaaatgacaaaagtattattgttcggcggtttgtttgtttgcctgggggcggtcgcagggccgtcgatgttgtggcaaaagtggcccactcatctctctttttttttgtctagatcgtcctggggcaactggtcggttctcttggccagcgatcgcgaagagggattctggctggacgatattttaattattagttacttatcccgtcttagcacacaatctatgatgaacatacgtaggtacagagcagccagatgactcccgtttttgggcatccgaaacaggagaaatgggaatgccgccaccttcgatcaagcgtcggccggtgggagtccgatctccacgacccagggcaagtgtggcccgttgctctcgctcaggctctggacggcggcgttgacgaatagagtttgccggcccggcgtcaggggatgaactcgaagcgggtccgaaccgtagtggcatgtcgcgcggtagccaagttgtttgagtgccatgagtcgctcttctttttcgagtacttcccgggtcgagtcccattttgtcgggccgagcgtggccagggactcaatcgcggtcgaagcgccgttatcgatgtcggagaagtgcgagggcgattcgctggccttgtcaccgcgccaggtgataagtttggagccccagcctttgtggatgtggccgaagcagtgcagacgcggacgggaccgtgctacggcggcgaagagctggtcgcagccgccgcgctgcttggaggcagtgagatccagaacgcccattgggggcccgtgtgtgatgacgacatcggcaccgtcaagagcgaagttatgctcgtcaccgcgtcggtactgaaacccccagtcagcttcgagtgacggcgtatatgggctcgcgtatacggtgagcctcgcgccgttctggaggttgaaatgatgggtgccttcgtccagcaagatgatatttgacgatgctgcttcggcaaacagctgccgtgcttcgccaaaatatccgtactcctgccgcaccaggtctggcgcaatggaaaacgtggagatggcatggttaaccttcttttggaaagcggctatgtcgagggtgaagtcgtggttccctgcaatgaccagcttaatcggggccttaatgcttttgagaagctccagtgatgtccggaattcgctcagcttggactcgtcggtcagatcgccgcagtgtatcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 22 - DNA, 8017 bp, Thermothelomyces heterothallica
  source 1..8017: mol_type = genomic DNA
agggtaggtgggatgggcggggtgtagggtaggtcggtgtagggtaggtcggctgggcggggtgtagggtaggtcggttgggcggggtgtagggtaggtcggttgggatgggtgtagggtaggtcggccgggtgtagggtaggtcggctgggcggggtgtagggtaggtcggtgtagggtaggtgggatggggcgctatgtgcggccgcgagctcgcgagcccatttttagcgaaggccatacaaacgagttttgcggaacccgggattccacccccgaagccgccggcgcgtgcgccccgctgcgcatcggtcggtgggtatatgagaagggggcgggcaagccggaagccagaggcaactgctactgttagctgccgctggcctccgcggcccagggcgcggcacggctgcgttgaagtctcccagtctcccacccgttggctgcgcggatccgcccgtcttggtggttgcgagctcgcgagcccatttttagcgaaggccatacaaacgagttttgcggggcccgggattccaccccggaacccgccggcgcgtgcgccccgctgcgcatcggtcggtgggtatgtgagggaggaagaagaaaaaaaaaaaaagctcctgcgggggggctgtcgggcacgcctactttcgggcgacccggcacctctccgcggcagccttcgcaggccgctgttggtcccatttcatacgtcgccgccttcgcgtggtgccctacggtctgccggggtaccgacgattgcggcgagcaccgcctcagcaccgctgctgccaccggcgcgacctcgcccgggggtgcgcgcggcatctgggaagactctgcaggcgtaagggaataccccatgtgcgccgaggggtgggctatgtgggtgcttggcggttcgccagacctttctaaagccaccgggggtacctaccggttggggacgcctacagggctgaacctcccggtcgggcctcctcttggggcgcttaggcggcgacttcggggcgcgatcgctccccgctctcgcccgccgacggcgctctggggaattcaggaggggaaagcagatgtgacccgcggctcgaccggcgcattgccggacgagctgcgcggccacgcgggcccccgcgcccgccgacccagtaacttagtgaactcttccgccctgaaacacgggcggttggccctaaccggctcacgatagttacctggttgattctgccagtagtcatatgcttgtctcaaagattaagccatgcatgtctaagtataagcaattatacagcgaaactgcgaatggctcattaaatcagttatcgtttatttgatagtaccttactacatggataaccgtggtaattctagagctaatacatgctaaaaatcccgacttcggaagggatgtatttattagattaaaaaccaatgccctccggggctctctggtgattcatgataacttctcgaatcgcacggccttgcgccggcgatggttcattcaaatttctgccctatcaactttcgacggctgggtcttggccagccgtggtgacaacgggtaacggagggttagggctcgaccccggagaaggagcctgagaaacggctactacatccaaggaaggcagcaggcgcgcaaattacccaatcccgacacggggaggtagtgacaataaatactgatacagggctcttttgggtcttgtaattggaatgagtacaatttaaatcccttaacgaggaacaattggagggcaagtctggtgccagcagccgcggtaattccagctccaatagcgtatattaaagttgttgaggttaaaaagctcgtagttgaaccttgggcctagccggccggtccgcctcaccgcgtgcactggctcggctgggtctttccttctggagaaccgcatgcccttcactgggtgtgccggggaaccaggacttttactctgaacaaattagatcgcttaaagaaggcctatgctcgaatacattagcatggaataatagaataggacgtgtggttctattttgttggtttctaggaccgccgtaatgattaatagggacagtcgggggcatcagtattcaattgtcagaggtgaaattcttggatttattgaagactaactactgcgaaagcatttgccaaggatgttttcattaatcaggaacgaaagttaggggatcgaagacgatcagataccgtcgtagtcttaaccataaactatgccgattagggatcggacggcgttattttttgacccgttcggcaccttacgataaatcaaaatgtttgggctcctgggggagtatggtcgcaaggctgaaacttaaagaaattgacggaagggcaccaccaggggtggagcctgcggcttaatttgactcaacacggggaaactcaccaggtccagacacgatgaggattgacagattgagagctctttcttgatttcgtgggtggtggtgcatggccgttcttagttggtggagtgatttgtctgcttaattgcgataacgaacgagaccttaacctgctaaatagcccgtattgctttggcagtacgccggcttcttagagggactatcggctcaagccgatggaagtttgaggcaataacaggtctgtgatgcccttagatgttctgggccgcacgcgcgctacactgacagagccagcgagtactcccttggccggaaggcccgggtaatcttgttaaactctgtcgtgctggggatagagcattgcaattattgctcttcaacgaggaatccctagtaagcgcaagtcatcagcttgcgttgattacgtccctgccctttgtacacaccgcccgtcgctactaccgattgaatggctcagtgaggctttcggactggcccagagaggtcggcaacgaccactcagggccggaaagttatccaaactcggtcatttagaggaagtaaaagtcgtaacaaggtctccgttggtgaaccagcggagggatcattacagagctgcaaaactccctaaaccatcgtgaacgctacctagaccgttgcttcggcgggcggcgccctcgcgcgccccccctggggcccgcaccgcgggcgcccgccggaggtacaccaaactcttgatatgttatggccactctgagtctcctgtactgaataagtcaaaactttcaacaacggatctcttggttctggcatcgatgaagaacgcagcgaaatgcgataagtaatgtgaattgcagaattcagtgaatcatcgaatctttgaacgcacattgcgcccgccagcatcctggcgggcatgcctgttcgagcgtcatttcaacccatcaagcccacggcttgtgttggggacctgcggctgcccgcaggccctgaaaaccagtggcgggctcgctagtcacaccgggcgtagtagcatacgacctcgctcagggcgtgctgcgggttccagccgtaaaacgaccttcacaacccaaggttgacctcggatcaggtaggaggacccgctgaacttaagcatatcaataagcggaggaaaagaaaccaacagggattgccctagtaacggcgagtgaagcggcaacagctcaaatttgaaatctggcttcggcccgagttgtaatttgcagaggaagctttaggcgcggcaccttctgagtcccctggaacggggcgccatagagggtgagagccccgtatagttggatgcctagcctgtgtaaagctccttcgacgagtcgagtagtttgggaatgctgctcaaaatgggaggtaaatttcttctaaagctaaataccggccagagaccgatagcgcacaagtagagtgatcgaaagatgaaaagcactttgaaaagagggttaaatagcacgtgaaattgttgaaagggaagcgcttgtgaccagacttgcgccgggctgatcatccggtgttctcaccggtgcactctgcccggctcaggccagcatcggttctcgcggggggataaaggcccggggaatgtagctcctccgggagtgttatagccccgggtgtaataccctcgcggggaccgaggttcgcgcatctgcaaggatgctggcgtaatggtcatcagcgacccgtcttgaaacacggaccaaggagtcaaggttttgcgcgagtgtttgggtgtaaaacccgcacgcgtaatgaaagtgaacgtaggtgagagcttcggcgcatcatcgaccgatcctgatgttttcggatggatttgagtaggagcgttaagccttggacccgaaagatggtgaactatgcttggatagggtgaagccagaggaaactctggtggaggctcgcagcggttctgacgtgcaaatcgatcgtcaaatctgagcatgggggcgaaagactaatcgaaccatctagtagctggttaccgccgaagtttccctcaggatagcagtgttgtcttcagttttatgaggtaaagcgaatgattagggactcgggggcgctttttagccttcatccattctcaaactttaaatatgtaagaagcccttgttacttagttgaacgtgggccttcgaatgtatcaacactagtgggccatttttggtaagcagaactggcgatgcgggatgaaccgaacgcggggttaaggtgccggagtggacgctcatcagacaccacaaaaggcgttagtacatcttgacagcaggacggtggccatggaagtcggaatccgctaaggactgtgtaacaactcacctgccgaatgtactagccctgaaaatggatggcgctcaagcgtcccacccataccccgccctcagggtagaaacgacgccctgaggagtaggcggccgtggaggtcagtgacgaagcctagggcgtgagcccgggtcgaacggcctctagtgcagatcttggtggtagtagcaaatacttcaatgagaacttgaaggaccgaagtggggaaaggttccatgtgaacagcggttggacatgggttagtcgatcctaagccatagggaagttccgtttcaaaggggcactcgtgccccgtgtggcgaaagggaagccggttaacattccggcacctggatgtgggttttgcgcggtaacgcaactgaacgcggagacgacggcgggggccccgggcagagttctcttttcttcttaacggtctatcaccctggaaacagtttgtctggagatagggtttaacggccggaagagcccgacacttctgtcgggtccggtgcgctctcgacgtcccttgaaaatccgcgggagggaataattctcacgccaggtcgtactcataaccgcagcaggtccccaaggtgaacagcctctggttgatagaacaatgtagataagggaagtcggcaaaatagatccgtaacttcgggataaggattggctctaagggttgggcacgttgggctttgggcggacgccctgggagcaggtcgcctctagccgggcaaccggcggggggcttccagcatccgggtgcagatgcccttagcaggcttcggccgtccggcgcgcggttaacaaccaacttagaactggtacggacagggggaatctgactgtctaattaaaacatagcattgcgatggccagaaagtggtgttgacgcaatgtgatttctgcccagtgctctgaatgtcaaagtgaagaaattcaaccaagcgcgggtaaacggcgggagtaactatgactctcttaaggtagccaaatgcctcgtcatctaattagtgacgcgcatgaatggattaacgagattcccactgtccctatctactatctagcgaaaccacagccaagggaacgggcttggcagaatcagcggggaaagaagaccctgttgagcttgactctagtttgacattgtgaaaagacataggaggtgtagaataggtgggagcttcggcgccggtgaaataccactactcctattgtttttttacttattcaatgaagcggggctggattttcgtccaacttctggttttaaggtccttcgcgggccgacccgggttgaagacattgtcaggtggggagtttggctggggcggcacatctgttaaaccataacgcaggtgtcctaaggggggctcatggagaacagaaatctccagtagaacaaaagggtaaaagtccccttgattttgattttcagtgtgaatacaaaccatgaaagtgtggcctatcgatcctttagtccctcgaaatttgaggctagaggtgccagaaaagttaccacagggataactggcttgtggcggccaagcgttcatagcgacgtcgctttttgatccttcgatgtcggctcttcctatcataccgaagcagaattcggtaagcgttggattgttcacccactaatagggaacgtgagctgggtttagaccgtcgtgagacaggttagttttaccctactgatgaactcatcgcaatggtaattcagcttagtacgagaggaaccgctgattcagataattggtttttgcggttgtccgaccgggcagtgccgcgaagctaccatctgctggataatggctgaacgcctctaagtcagaatccatgccagaacgcgatgatactacccgcacgttgtagacgtataagaataggctccggcctcgtatcctagcaggcgattcctccgccggcctcgaagttggccggcggtaattcgcgtattgcaatttcgacacgcgcgggatcaaatcctttgcagacgacttagatgtgcgaaagggtcctgtaagcagtagagtagccttgttgttacgatctgctgagggtaagccctccttcgcctagatttcccagcgagagcccgccggcggaacagccgggcgagccttacgggggaagccttaaggggattgagaagtggtgccgtgcgttcgcgcgcccctaggtcctttagccggccgcaggtgtagggtaggtcggttgggaggatggggtgtagggtaggtcggtgtagggtaggttggttgggaggatggggtgtagggtaggtcggccgggtgtagggtaggtcggtgtagggtaggtgggatggggcgctatatgcggccgcgagctcgcgagcctatttttagtgaaggctatataaataagctttacgttaccgggccttgctaccctcgagtggcgtgggccgtgctgcctactgggcattgctcgccgggctgtataagggaggggtcggggtcgcggtctagggtaggtcgggtgggatggggtgtagggtaggagaagcgctctagtcgtgtgtctttttctctaggtctattattagtactggctgtagggcgacgtgccctgccttgttataatattatattgtatgtttaggcctatactagcttgtaatctatttgtatctggcttattaggtacggcttcctttgtatataactagagaggctctggtatgcttcttagtatagcggtataggattcataatcatagtaatgataatcataatagtaataataataataatagtaatgataataataataatctatttatatcttatttaaaatgcttgtacggctgcctgctcttaaggagtagctagatatgagatggtagggtagctagctaacctaggctagacgttctcgtcccttagctatataagtgctatatattatagttagttatctaacctaccttcttacttgagcagaagaggtagggttctagtatagctagtagggcttctaggcctaagggcctgttattcgagttattataggttagtatttaatatagttatagggataggcctcgattacgggtataggataggtaggataggtatagggtaggtcggttaggaggatagggtgtaaggtaggtcggccgggtatagggtaggtagtaggttaggcggggtgtagggtaggtcggtgtagggtaggtgggatgggcggggtgtagggtaggtcggttgggaggatggggtgtagggtaggtcggtgtagggtaggtcgg

SEQ ID NO: 23 - DNA, 20189 bp, synthetic construct
  source 1..20189: mol_type = other DNA
gtttaaacatggacaacttccgcgagctgccggcgtcggagctgccgccggggatgcactcgggcgccgagttcacgtccgtctgcatcaacacggccatctacctgccctggctggtcggccagtgcgcccgccacggcgtcgtcttcaggcgcgccgtgctcaagcacatctcggaggcggcccgcatgagccacacggggcggagggccgacgtggtggtgaacgcgtccggcctgctggcgtgccgcctgggcggcgtcatggacgccaaggtgcagccggcgcgcggccagatcgtgctggtgcgcaacgaggcggccggcgtcatgcccacgacgagcggctgcgacgacggggacgacgaggtcgtctacgtgatgcagcgcgcccacggcggcggcaccatcctgggcggcacgtaccagaagggcaactgggaggccaacccggaccccaacgtcgccgtgcgcatcatgaagcgcgcggtcgagacgcacccggagctgacgggcggcaagggcatcgaggccttggacatcatccggcacggggtcgggttgaggccgtggagggagggcggggtcaggctcgagaaggaactgatcgacgggacgtgggttgtgcacaattacggccacgccggttggggctatcagggaagttatggttgtgccgagagagtggtggagctagtggacgagatcgtagggaggacgaagtgcgagtcaaagttgtgaactcaaatggcatgccaatggtatactataaagaaaaatattggtataaccgaaccccaaaaaaaccacctcgaaatttgtgtttagttgtcaacttgattcaaggaaggagtggttagacaaagcatatgttaccaacacagtggaaggccagaacccagcacctgctgccacctgccaagagccgacgctttctcgcccaggtccctgtaattcataattacttgcattaagcttcgtgtcaagctccagcgcaacgcgtccacaacgcccgggcggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatggcggttgctcgccccgtgagggcgctggcgctgacgggcgttgtcctgtggtgtttctttgtatggcagcttttcaagccaacaggatcgcccaagagcccggccagggtccctctcctgtcgagagagcgcgatcctaatctagaccgtaggcactgaacagcccagggcggccttggaaaccatttgctgacttgaaatagccaccggcgagcccgagggtgttctgacatatgcgtccggggagtacgcacccggcccctccgtcagcgccctcgacggcgaccccgcctccctgacccgcgaggtcatccgcctcgcccaggacgccgaggtcgagctcgagcgccagcgcggcctgctccagcagatcggcgacgccctctcctcgcagcgcggccgcgtccccaccgccgcccctcccgcccagccgcgcgtccccgtcaccccggcccccgccgtcatccccatcctggtcatcgcctgcgaccgcagcaccgtccgcaggtgcctggacaagctcctgcactaccgcccctcggccgagctcttccccatcatcgtctcgcaggactgcggccacgaggagaccgcccaggccatcgcctcctacggctcggccgtcacccacatccgccagcccgacctctcctccatcgccgtcccgcccgaccaccgcaagttccagggctactacaagatcgcccgccactaccgctgggccctgggccaggtcttccgccagttccgcttccccgccgccgtcgtcgtcgaggacgacctggaggtcgcccccgacttcttcgagtacttccgcgccacctaccccctgctcaaggccgaccccagcctgtggtgcgtctccgcctggaacgacaacggcaaggagcagatggtcgacgcctcccgccccgagctgctctaccgcaccgacttcttcccgggcctgggctggctgctcctggccgagctgtgggccgagctggagcccaagtggcccaaggccttctgggacgactggatgcgcaggcccgagcagcgccagggccgcgcctgcatccgccccgagatctcgcgcaccatgaccttcggccgcaagggcgtctcccacggccagttcttcgaccagcacctcaagttcatcaagctgaaccagcagttcgtccacttcacccagctcgacctgtcctacctgcagcgcgaggcctacgaccgcgacttcctggcccgcgtctacggcgccccgcagctccaggtcgagaaggtccgcaccaacgaccgcaaggagctgggcgaggtccgcgtccagtacaccggccgcgactccttcaaggccttcgccaaggccctcggcgtcatggacgacctgaagtcgggcgtcccccgcgccggctaccgcggcatcgtcaccttccagttccgcggccgcagggtccacctggcccctccgcccacctgggagggctacgaccccagctggaactaaggcaggcaggagttggagtatgagggtagccgctgatggctattcttcccacgtttttgtgtgtttcctcttcatttttttttctcttgccgcaacatgacggctcctgtctctgaagggaacccctgaaattcagggttatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtcttaattaacgcgatgagcagagcattttaaaggaacgccgcactcacaaacaccaacactttagtgtctagtctacagaggcgtccctccccgtcttggatgcgtgattccattaccgtagatagtaccgcaaatgcacgggggtgtagtgtatgaaccacgctgggttcctgacctgacccggcaacccaatggagcagactcagggcccgctggccccggtggcgtatcaggtgactgttgggggagctaaccttggcaaacaaccgagctcagcgttaatgcatttcaagaagtcggtttgattgatcatccgcgaggaccgattatcgtacggcatcgaaaatcgtctcgccggagcgcacggattatttgaagaggctggcttgttgattgcaattgtcggctgccggccacgtcaccggccttgcagggcttatcagtaaatgcgggggcggagcagaggcggttcttgtcaagggtaggaggggtccggcaaagcccgagacggtggctgttcggaaacccaagaatggaccctgacagaacaattttcggattgggttcgttgcaaggatcgaacactacatcttccgagagagtttggaggttgtaagaacccttcgctaccgggagaacaaatcaccttgttgaatcagctctgtcactgctagtggcgagatggcctaagcagcgagactgttccccctgccccgctgtggatccgcatgactggtccattctggtcacttcgctccacttctctgcttttgcattgaccgctcagcggctgttgcgccttcctgacgcattcatagccccactcctgggcggcagcctggcgcttccaccatgcttgcccaacacgtatataaccttctcggcctaccctctaccacggagccactttctcttctccaacatcctccacacaacacccttctccttcgccatcaaagaggcatctatcggaaaatccaacatcgccagactcaccgaaacttcatacactcataacaactgcaaccatggccctcttcctgagcaagcgcctcctgcgcttcaccgtcatcgccggcgccgtcatcgtcctcctgctcaccctcaacagcaactcgcgcacccagcagtacatcccgagctcgatctcggccgccttcgacttcacctcgggcagcatctccccggagcagcaggtcatctcggaggagaacgacgccaagaagctcgagcagtccgccctgaactccgaggcctcggaggactcggaggccatggacgaggagtccaaggccctcaaggccgccgccgagaaggccgacgccccccgaaccggcggcgcccgccctccgcctcctctgggcgccagcagccagcctcgccctggcggcgacagcagccccgtcgtcgacagcggccctggccctgccagcaacctcaccagcgtccccgtcccccacaccaccgccctcagcctccccgcctgccccgaggagagccccctcctcgtcggccccatgctcatcgagttcaacatgcccgtcgacctcgagctggtcgccaagcagaaccccaacgtcaagatgggcggcaggtacgcccctcgcgactgcgtcagcccccacaaggtcgccatcatcatcccgttccgcaaccgccaggagcacctcaagtactggctctactacctccaccccgtcctccagcgccagcagctcgactacggcatctacgtcatcaaccaggccggcgacaccatcttcaaccgcgccaagctcctcaacgtcggcttccaggaggccctcaaggactacgactacacgtgcttcgtctttagcgacgtcgacctcatccccatgaacgaccacaacgcctaccgctgcttcagccagccccgccacatcagcgtcgccatggacaagttcggcttcagcctcccctacgtccagtacttcggcggcgtcagcgccctcagcaagcagcagttcctcaccatcaacggcttccccaacaactactggggctggggcggcgaggacgacgacatctttaaccgcctcgtctttcgcggcatgagcatcagccgccccaacgccgtcgtcggccgctgccgcatgatccgccacagccgcgacaagaagaacgagcccaacccccagcgcttcgaccgaatcgcccacaccaaggagaccatgctcagcgacggcctcaacagcctcacctaccaggtcctcgacgtccagcgctaccccctctacacccagatcaccgtcgacatcggcacccccagctaagaagtgtaagatgtaagagatgacgggggatttctcggtgtgtgaggaagtcacctgcgaacatgacatgcgggaatccatctttatggcggagggcgtctctggctgaatgatctagcaagggaaatgccttctatttgactttcccacacgataggtacgctcatgcttcgacataaatttgcgatgctactggtagcgtaccttaatgtacactagtctcgattcatatcaacggacgtggtctggtatggcaacttgactgccacactaatgttactggacccctaccggtccgacgttccaagcatcggaagctcttccaaaatgctttcttaataaggtttatttgcgtcgaattgcatacaactgcccctctacatcgtgctagcaaccttcgccttcattaattaagattcatcaactcattgctggagttagcatatctacaattgggtgaaatggggagcgatttgcaggcatttgctcggctagtcggaatgaacattcattccgagacctaggatgtgacggaatgaaggttcattccggactctagataagcacggaatgaactttcattccgctgaagcttgtcaatcggaatgaaggttcattccggctagtcggaatgaacattcattccgagacctaggatgtgacggaatgaaggttcattccggactctagataagcacggaatgaactttcattccgctgaagcttgtcaatcggaatgaaggttcattccggctagttctccccggaaactgtggccatatgttcaaagactaggatggataaatggggtatataaagcaccctgactcccttcctccaagttctatctaaccagccatcctacactctacatatccacaccaatctactacaattacaaccataatgcacgtcccaggcccattgtcctcgcggtggacggcctaccttggcctgctgtccgcattttctgcagcatttgcacctgcaggtgggtcccgcgagcatctgatgccaccagacgaccgagttgacgatagtagtcgccgtcaaggagcatgacttcaaaaagtgccatcaatcgggcttttgtaagcgcaaccgcgaattcgccgaccacgccctcgccacgagctcctgggcgtcaccctacaatgtcgcaccagattccggatcgttcaaagatggccagtaccaggctgtgatcttgaagaccaccaacagcggcgagacggttcggcttcccattaccgtctctttccttgagtctggtacagcgcgagtcacggtggacgaagagaagcgccagaaaggagagattgagttgcgccatggcagcaaagcgaggaaagagcgctataacgaggcagagaaatgggccattgttggagggatgactttggacaaggaggccaaggtcgattatgaggacaagacgcagattaccgtcaagtacgggccaacctccaagttcgaggcaaccatcaaattctctcccttcagcatcgacttcaagcgggacggcatcagccacgtcaaactcaacgaccagggcctccttaacatcgagcactggaggccaaaggttgacaaaccggagcccgagaagaaggacggcgattcgactgaagagaataaggcagaggaagcaaaggaggagcccaagggagaggatgagtctacctggtgggaggagacatttggcggcaacaccgataccaagcctcgcggcccggagagcgtcggtcttgacatctccttcgtcggctacgaacacgtctacggcatcccctcccatgccagctcgctgtcgctgaagcagacccgtggcggcgagggcaactaccaggagccgtacaggatgtacaatgctgacgttttcgagtacatcctcgatagcccaatgactctctacggctctattcctttcatgcaggcccatcgtaaggactcgagcgtgggcgttttctggctgaatgccgcggagacgtgggttgacatcaccaaggcgaaagactccaagaacccgctctctctcggcggcaaggccaggaccaacacgcatacccactggttctccgagagcggtctgctggacgtttttgtcttcctcggcccgacgcccaaggatttgaccgccaggtacggtgagctcactggcaccactgcaatgccccaggagttcgctctcggataccatcagtgccgttggaactacgtctcggacgaggacgtaagggatgtggaccgcaagatggacaagttcaagatgccgtacgacgtcatttggctcgacatcgagtacactgacgagaagaagtactttacctgggacaaacactcgtttacagaccccatcggcatgggcaagcagctggacagtcacggcaggaagctggttaccatcattgatccgcacatcaagaataccgacaattaccccgtcgtggcagagctaaagtccaaggaactcggcgtaaagaacaaagatggcaacctcttcgagggctggtgctggccaggctcgtctcactggatcgacgccttcagtccggctgcgcgcgagtggtggtcgtcgcttttcaagtatgacaaattcaagggcaccatggaaaacacgtggatctggaacgacatgaacgagccgtcggtctttaacggtcccgaaaccacgatgcccaaggacaacctccacgacggaaactgggaacaccgcgacgtgcacaacctgaatggcctgacgttccacaacgcgacctaccatgcgctcctcacccggaagcccggcgagctccgccgcccgttcgtgctgacccggtccttctttgccggctcgcagcgggtcggcgccatgtggaccggcgacaaccaggctgcctgggaccacctcaaggcatcgattcccatggtcctcagccagggtatttcgggcttccccttctcgggcgccgacgtcggcggcttctttggcaacccggagaaggagcttttgacgcggtggtaccaagccggcgccttctaccccttcttccgggggcacgcgcacattgatgcgcgacgccgcgagccatacctcgccggcgagccgtacacgaccatcatcgccgcggcgcttcggctgcgctacagcctgctgccgtcgtggtacactgcgttccggcacgcccatctggacggtacgcccattatcaagcccatgttctacacgcatccgtccgaggaggccggcctcgctatcgaggaccagttcttcgtcggcaacacgggcctgctcgccaagcccgtcacggagaaggagaagacgacggtcgatgtgtggatccccgacggcgaggtctactatgactactttacgtaccaggtcatcccgacagtcaagggcaagacggtcacgctggatgcgccgatggagaagatcccgctgctgatgcgcggcgggcacgtgtttgcgcgccgcgacgtgccgcggcgctcgagcgcgctcatgcgctgggacgactacacgctcgtcgtgacggtgccgcgggaaaacaaggtcgccgagggcgacctgtacgtcgacgacggagactcgtttgagtaccagaacggccagtacatccaccgccggttcgtctacgacggcgcggccaagagcctcagctcggtggatgccgagggccgcgacgcggcctcgatccgggagggcgcgtggatgaagcagatgcgctccgtgggcgtgggcaagatcgtggtggtcggcgcgccggccagctgggcgggcaagaagagcgtcaaggtagagagcgagggcaaggtctgggaggccaggatggagtttacgcctgctgggcagggcagagccgcgttcgcggtgatcaagaaggtgggcgtgaggatcggggcggattggaaggtcgagtttcacgacgagctctgaggccctatgacctcccgagtcctggtcgagagaggggagctggagagagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtaacccaaagtaataagtctgtagtaattggtctcgccctgaattccaaactataaatcaaccactttccctcctcccccccgcccccacttggtcgattcttcgttttctctctaccttctttctattcggttttcttcttcttttattttccctctcccatcaatcaaattcatatttgaaaaaaattaacaatggagtccacacccacgaaacaaaaagctattttttctgcctcgctccttctgttcgccgaacgcgggtttgacgccactacgatgccgatgatcgctgaaaatgctaaggtcggcgcaggaacgatttaccgatactttaagaataaggagagtctggtcaacgagctgttccagcagcacgttaatgaatttttgcaatgtatcgagagtggcttggcgaacgaaagggacggttatcgcgatgggttccatcatatcttcgagggaatggtcacattcacaaagaaccatccgcgcgccttgggatttatcaagacacattcccaaggtacattcctaaccgaagagtcacgccttgcataccaaaaacttgttgagttcgtctgcaccttctttcgagagggacagaaacagggcgtaattcgaaacttgcccgagaatgccctgatcgccatcctattcggatcgtttatggaggtctatgagatgatcgaaaacgattatctctctctaacggatgagttgcttacgggggtagaggaatcgctctgggctgctctctcccgacaatcggctagccctcccaagaagaagcgcaaggtcagcacggccccccccacggacgtctccctcggcgacgagctccacctggacggcgaggacgtcgccatggcccacgccgacgccctcgacgacttcgacctcgacatgctgggcgacggcgacagccccggccccggctttaccccccacgactccgccccctacggcgccctggacatggccgacttcgagtttgagcagatgttcaccgacgccctgggcattgacgagtacggcggctgaggccggccgcgatacccatcatcaacacctgatgttctggggtccctcgtgaggtttctccaggtgggcaccaccatgcgctcacttctacgacgaaacgatcaatgttgctatgcatgagcactcgactatgaatcgaggcacgttaattgagaggctgggaataagggttccatcagaacttctctgggaatgcaaaacaaaagggaacaaaaaaactagatagaagtgaattcatgacttcgacaaccaaatcatcttgtctccgtctgcatacgtgaagcttgtgacgattattctcgcgatgccacgacaaaggttgtgcgaccgtatcttgtccactgtcgtccagtctgcctattccccctccagtgctgccatgtgtcgtaccttgaggtaggtagtctacctaggccagggagctgttagtgcccggctactgggtaatttgtagcgctggagcgattcggtcacaggcgtcaagagtgctgtagcaatgtccgacgccattgatcctgatatcaaataccacctgggcaggtctgggtatgtgaggtcttgtcggatgtgtcgagttcttctccaacgtagtgttcattcgcgctcatttaaatggcgcgccgttggcggtaacagcaggtgccgttcgctgctgctcggtccttttcccgaaagtaccctgccttcctggcctcgacctccgcctgcggggctctgggcactgtcaaactttcgcagttttgcacccgcagcgattcgagcgggacccgcttcgccaacgcggatcaggagaggtaaaagtctcccctgtcctttctgccggagcatcctccgtccgtcgttcgctggttgttgtgtgtgtgtgtgtgtacagtactgtaggtacatagtgctcaccccagggcatttccccaatccgcgattgcgcataccataggcgtcatctgaatctgcgttacgtccggataacactacgcagtacgaaggagtcggtgctactacgcccgcaagttcaagtctacggtgtggctaaggtgctcaagggcatagttacacgcaccccgggcgagcttctcgcacctcgaccctttgccggggcgtcactggtccgacaactcataatccaccatcgcttgggcaggcctgcatcccccgtgggtcgcatccaagccgccggtaaataccggccgacacacacacacacacacgcaccctgtctagcgcgacgctacaaaagagaccggcaggcactccccgcgaccgcttgttctggccatcccgtctctcgcctctgcagttatcgcctgcctcatctctgcgtcctgataacttttgtcacctctgatccccccccgacgagacggtctcgccgaccaggacccgagatggcggaccgccatcctactctgtcccagtccttcgccgagcgggcgaagacggctagccacccgctcacccgctacctctttcggctcatggacctcaaggcctcgaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 24 - DNA, 18731 bp, synthetic construct
  source 1..18731: mol_type = other DNA
ggcgcgccgtttaaacgaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagggcgacggcctcggccagcagtacaactcgcccagcaagttgatcaacatttgcggcaccgacattgtcatcgtagggcgtggcatcaccgccgccggcgacccgccctccgaggctgagaggtacaggagaaaagcctggaaggcctatctggcgcgtctggcgtgatttggggggagggggagaggagatgggggacgggaggggtcgccttggtcagtcttgtgcgtgtcctgcagcggattcgtcaccggggcagcacccaaaagagggagaaaaaggggaaaaaaaataaataaataaaaagggttaagttgttgaaaaaagtgttgtgagctctctggcaaggcgcgccatttaaatcgttggcggtaacagcaggtgccgttcgctgctgctcggtccttttcccgaaagtaccctgccttcctggcctcgacctccgcctgcggggctctgggcactgtcaaactttcgcagttttgcacccgcagcgattcgagcgggacccgcttcgccaacgcggatcaggagaggtaaaagtctcccctgtcctttctgccggagcatcctccgtccgtcgttcgctggttgttgtgtgtgtgtgtgtgtacagtactgtaggtacatagtgctcaccccagggcatttccccaatccgcgattgcgcataccataggcgtcatctgaatctgcgttacgtccggataacactacgcagtacgaaggagtcggtgctactacgcccgcaagttcaagtctacggtgtggctaaggtgctcaagggcatagttacacgcaccccgggcgagcttctcgcacctcgaccctttgccggggcgtcactggtccgacaactcatttaaatcatttccacaactcatgccgagagaagtgtcagactgggcaactaaagtagtagtagtaatagctcgattaccatgatgaaatgctgggcgtcgaagcagctgcaggtccggcatgcagcaatccccactccgctccatcggctgctgttctggggtcaatccgtaccctcccaagttcacctcgccgctgacctcgggatcagctgcgttgtgcattcatgaataatgcgcaacatgagcaacccaacttcatcaagggagtttcgacgtcaccatatcaccacacatcttggaacagaattggggacaaggcagctggattgacggggaaatacataaaccggacgacctatgacgaccatcctatcccgtcaccgactccgatcccctgcggatgatggctcaaagaaccaagtttgatgagccggctgtgtgtcccagcacatacaacaaccgaagtatcagccccgttccgaacgcaggatcccagtctaccgaatcgattttggacagcccgagagaagccaaacaccgaaccgaaggaggaattgtcggaagacgtgcattatgagtcgttcacgcgcgcgttagcggcggttctccgactcgcgcatccggcgcatgtactcctcctggtagctgccgacgttgttcttgcggtcggcgttggtgacctggtcgaaggggacgcggtgggccagcatctcctggatctccttggcgggcgggtactggcccgggcagatccaggagccgggcgggtggcagacgcggttggccgggtcggcgacccacttcttgctctcggcggagacgttcatgaccttgaagatgcggaccaggccgtacttggaggagtagacttcctggaagagcgaggggttgaccttgacgcccttgcgcttgccggcctcgtgcaggttgtacaggagggaggcgcgcatcatcggggtggggcggctgtagtcgttgcggtggaagccgaactgctggcacagcgggtcgtccgggcagatgtcgtggtagacggagttgccgatgcgggccatgtgcgggctcttcatcaggtcgcccgactggccggcccagatgaggacgtagtcggccatgtggcggaccaggctgtgggcctcgacgaccgggctggtgagcatcttgccgatggtggcgatgtgctcgtggttccaggtgttgccgtcggccagcgaggtgcggttgccgatgccggtgatctggtagccgtagtcccaccaggcgaggacgcgggcgtcctccggggtgctgtcgcgcagccactcgtaggccttcaggtagtcgtcgacgagcaggttcatcggcttgccggtggcgcggttctggacgacggcggcgaagacgatcatggggttgctggactgctcggcgaacttggtcgagtggctggcgaactccgagctgaagaagctgacggcggtggtggtgaccagggcccacatggcgatggacaggaccatgcggtggccccaggccagggaggagccggcgaagacgtcgcagaaggcgcgggcggtggtggcgttcttggcgtcgtcgcggcccgagcccttgccggcgcccctctggtggcgctgggcctgcttctgctgcttcttggccttggtggcgtcgctgtcccagaaggagagctggacggcggcctccaggatggtgccgacgaagatgccggtcgacaggcaggcggcggggcccgacaggagcaggaggcgggccatgcgggtggagaagtagtagacggcgcccgagttcaggagccagaagaccttggagggcgagtagtggacgaaggtcgagacggccaggacgatgctgcccaggccccaggtgacgccgcagacgtggaggaaggcccacatggcctcgggcgaggccggctggtgctcggcgacggagtcgacgagcgggttgccggtgcgggtgtgctcgacgaagagggcgcggacgcggacgctcagggggccgaagtagccggtgggggcgaggaccgagatggccagggcggcgacgccggccatgacgctgaagacgcggacgcggatcttgaagttggcgcgggagcggacctcgacgccggcgcgggcgcggaggacctcgcagacctggaggccgcacaggaagacgaggaccaggagggcgcccagctgctccagggacttgaacgggctcatgccgacgggcgggacgcagacggcgatggcggtgccgacgacgtagaagagggtgtaggcgcgcaggaggctggggttgtaggtgttgcgggcccagtcgaccatgctggagatgccggcgtgcatggcgaccatgttcaggacgaagatgtagccgccccaggcggcggccatgtagccgtaggcgacgccggtcaggacgccgatcggccaggagctgcgggtgcggagggagcggacccagcagtagaaggtcaggagcatggcggcgacggcgatgcactcgttgtcgaactcgccggccatggagcgcatgaggtgggccgggatgatgctgaaggacagggcggcggcggcggcggcgacggtggagcccgaggcctcgtaggtgcagaaggcgagggtggcggtggcgatggcgccgaaccaggcgggcatcaggacgcagacgttgttcagcgacatgggcatgccggcggcggccagggcgcggtggatggcgacggcggtcagctggaggcccgggtaggtggtgctgccgacgggcctgccgagcgggtaccaggacatgtagtcgaaccaggagaagaaggccgaccagccgtgggtgctcatgtactcggcggcgcggtagttgaaccaggggtcgaactcgtggatgaggtagccgtagatctggacggagatcatgcggacggtgaaggcctggaagcaggaggcggccaggacgaagagggcgacgacggtcaggacgaagtggaccggccagaacgggaaggggaagatgccgatgaagtccttctcgtcggtcagggtcttgggcaggaggatgaccttggcgggcgggctggcggtcttggtctgggaggcggcgtcctcggcctgggcggaggcctcgcggctggcggtggcggcggagccggagtcgcccagggagttgcccttgcgcttgcccatgttgcttgcgtggtgtgctggaagctgagtgtattaggtggattgacaagtccctgcgggcaacgggaccgagtgagcaagccaggatcaggcgagcaagaggcaggtggtctgattctatcaacctacgtttagagacttgagatggaccagggaatgggcgttttgttttcgaattgatggtttacgatggatttcgttggacggaagaccgatgaggggaaaggagaggagaagcccaaagagggggtcggaggtggcctttattaagaggcggccggccgggcaatgggcagatcagccatctttgctgcatcgttcctgcgactgtttcgtcagaccgggcggggtaatgtcaggagagctccctggtaggttgcgggcagcggcagtgatcacgttgactggctcacgggatcgcgtgacgagtacatcatgatggcacgacctcgcaggcgagccctgcgtggcttaaacaggccaaggtacctggcccgagcgtcctgcagccagcgctaacagcccagcccaagccagaatctggggtaatctggggtaccggggtgcccgacccactgcgggcaaccagcgcttgtgcaccgcgtaaggcctcaacaagacatcagttagtatcgatgccgagattcagttggcaattacatacgtctaacttttccaatgcttattttgagtttcttgtagttatgcagctggtggaagttaggacaggaaccttgagtgacaagcaatccggccgggccgggaaggtgcccgctgcacgatcagtggggcaaaggtggggtatcccgagcaggagcgaactccaacagagtattcgatcaaaaaaggcaagtcctcccccaccatcctttgtagcttgcaatgcatctccttttttgcaatggattttgcttcgcgagtgctaatgccttgtgaaggactatgtggttggttcaaacctgttgttttgatccatctagtccacgttgcaggcatacaaatacccgacttaattaagagtgtaggatgatggtggcaaaactttgaccgacgagacgagacgtgttcacggaggcacgggaggtaatgaatgctctgcttctcgaagcttgcgctctcttgcagcaagctgcatcatgaggatcattgacgtgctggcttgtcggcaacaacctgcattgtcggaggaaggatgtgaaaggcggttacgaagccggaacatccaagtttgcattgccaaggctaggacggagcaggaactgtttttcacatgcaacttccgaaagcgggggagacccccgccatcccataggcatcaaatgtcatcgcaccacgattacgcagcatcaaacgaacaataatcgggggactccaaacgagtctattcatccgtggtatctccatgttaccgtgtcattaatggtaaaccccaaatttccagcggcagccaatggttggccgtcccaaacccgctaaaacgctcccaggcccattcccgtccgatgcagacactttagagctcgtcgtgagcaaggtggccgccccgtcgtgatgatgaacggatgctaaaggggtgcgcctccgtgttaaagacaaatttgttcccgccgttggcctgcacctgcacatccgactcctccgcaaagatcaggtacgcatacttgagcgcctcggcaaaccagaagctctccatatcgtcagaggcacccccgccgttggcctgcgtcacgtcgttgatggacgagtacgcgctgccggcgcggcatgcgtcctcaatggcactgaacgcttcccacgccaggtcctgccacttggagtcgcccgtgacgcggtatgcgtagtacaagctctccagcgtctccggccgcaggatgtaatacggtgccgtcacccagaatcctgccgacgagtagaacccggactgggacgagggcggcgagccgccggcgcccgtcacgctgtccacccacgcgaagccttcggggccgattccagaagccgtctggttgtacgtggcaaagtacgagctggcaagcttgattccaaagtcaatgtacttttgctcgttcaggagaatgcctcccaagatgaagttgccaccggcaaaactggccactacaaggcaagaaaagggggagactgttagcacacaatcaccaccacgtacgacgtctcgagatgactgtcgaatggagaaacttacaatgtcctgagtttggcgacgtagactgtccgttgtacgaagacaaaaaggtcaagtccttgcgcgtcgacgggtgagaggcgagatgcgcaatggtcgagtcggcagcaaggacccagcgatccttgtagtgtgcaaacgcaaccgggtcgtacaggtacatcttgatcaggtactcgtagaagctgtccatgaggccggaccagctgccgctgctatcctgaaaggtaccgttgctcgtgctgacaaacgttccaatcaggccaggccatgcctccgggcttccctttggattcaggagatacgactcgcccttctgcgcaagctgggcatactgcgggtttcccgtcaggtcgctcaaccgtgtccactcgagcaccaggcttccaatttcagcgacgttgttgctagatgcaccacttctccggacagtagggttgaagaagacggtagggtccgggacaccgctgggagtggtgaacgcaaccttgaggccgttggccagtgtttgagcctgcctcagaaggctgtttaccagggtctggtttgtcgccaaggagctgaaaggacctcgcaacaggtcataggctaggtagaatcatcgattagccataccgtcagcgccgaacaatccttgacgcggaaactaggctcgcccatcgaacacgtaccagaaagcaggccaccgaggtaccgaatgttggtctcgaacacggagatgccttggttggcaaccgcagtcgtggtgaagttgatctgcggtacatactgaaggatcgtgttcacaatgtcggcatcccccatgaggatagccgtgtccaagccatcgattgccgacgagccccagccgtttctaggccaaagtaagatacaaattcgccactttatgaaaggactgaggttccgtcacgagctcgcagcttacctctcatcatcaaagctgttgctgaccgggtggaggtcgtcatggggaaaggcaaaatggtggtaagcgttccacgacgtctggaatgcggccttgactgctgccgccctcgtagggttgggagatccacgttttgtggcgcccggctttggatacgccagcgcaggtccgatgagcccgagggcaaggacggagctgctagggaatctcatggttgcagttgttatgagtgtatgaagtttcggtgagtctggcgatgttggattttccgatagatgcctctttgatggcgaaggagaagggtgttgtgtggaggatgttggagaagagaaagtggctccgtggtagagggtaggccgagaaggttatatacgtgttgggcaagcatggtggaagcgccaggctgccgcccaggagtggggctatgaatgcgtcaggaaggcgcaacagccgctgagcggtcaatgcaaaagcagagaagtggagcgaagtgaccagaatggaccagtcatgcggatccacagcggggcagggggaacagtctcgctgcttaggccatctcgccactagcagtgacagagctgattcaacaaggtgatttgttctcccggtagcgaagggttcttacaacctccaaactctctcggaagatgtagtgttcgatccttgcaacgaacccaatccgaaaattgttctgtcagggtccattcttgggtttccgaacagccaccgtctcgggctttgccggacccctcctacccttgacaagaaccgcctctgctccgcccccgcatttactgataagccctgcaaggccggtgacgtggccggcagccgacaattgcaatcaacaagccagcctcttcaaataatccgtgcgctccggcgagacgattttcgatgccgtacgataatcggtcctcgcggatgatcaatcaaaccgacttcttgaaatgcattaacgctgagctcggttgtttgccaaggttagctcccccaacagtcacctgatacgccaccggggccagcgggccctgagtctgctccattgggttgccgggtcaggtcaggaacccagcgtggttcatacactacacccccgtgcatttgcggtactatctacggtaatggaatcacgcatccaagacggggagggacgcctctgtagactagacactaaagtgttggtgtttgtgagtgcggcgttcctttaaaatgctctgctcatcgcggccgcgcacacctacttgtatacactcccaagcgtagccccaagagcaggaaaagaaaagtgagaaacaagaagagatttgctgctcagagcaacccccagctgcaccctccccaccactcgcgtctccaatttagtggttgccgtgggctcataggttggggtccctatggatttgggggtggacgctgcaggatcggagtaatcatcgaggaggacgacgctgcttgacaggccaggcgtaaaaagaggcaatcatctgatggtgtggtgtgagagaacgggtcaagtaacagtcgggggaacgttggtctttggtttgggttgaatggtacagttgattaaaacaaaaagacagtgacaaggggggaatattctaccaaacaacgtccgcacgacccgcgaggctagccaagaacgcacgacccaaaccacgtccgcagacagggaaaacgagacaaatcacggacaacaggagagaccagccgatttttgttgttgttgttgttttactgcagcctgcggtaggacttgcagagctcgtggtcgcggatgtcgccccagccaccgttcttgcgcgggggcgaaatggcggccatggggaacttctcgccgatgaccagggtctcggggaagaggtactgcttgttgttgttcaggaggctctcgatctgggcggactgggtgctggggcggcaggtcttcttgtggtgcatgccgcagtcgccggcgtggaagatcctgggggcctgcgggaccaggaccttccagaccttggggaggcaggccagggtgaggtactggagggtccagtcccagttgtagtcgtcgtaggtgcagaaggtgtcggtgcactcgatcagcttctggtaggcgtcgcgggtcagggccaggcccatgttgtgctcggtcgacttccaggtcttgacgtcgaccttgtcggcgatgccgtagaaggagcggatggtggtgtaggtgcccagggagaggacgtcgcagccggggcactcctgctgcttgagcttccacatcttcttgaagacgtggtagaagtcgggggccaggtagtggtcttcctccaggaagaggatcaggccggtgtagtcctggaggaccttgacgcgctcccagacgaagtgcagcttccaccaccagtggtgcttggtctgcgagaacttggcctcgcggtagtggccgaagctgtcggggtactcggcgttgatgcagccgagcttcagggcggcgttcttcttgaggtcgcgggggcagtcgcgggggtccgagccggggaactcggaggggtagagctggatgctgaacgggaagaagacctgcaggacggggcagaagtcgacgctggagatgagcgagttgatctcggcgctccagaagtcgtgggagaagatgacgaggacctcgcggatgccctgggccttgcggagcgagtcgatcaggaggcggaggtactccgggcggttgtggacctggacgacgaggaccagctcgcccggggaccaggtgccgtccttgtcgacgttgcggagcatctggtcgaagttcagctggtagacgaggctgcggtagcggagggtcaggttgtcgacctccgggcgggggacggcggggacgagcggggcggccgagtcgttcgagaccctgcggatgccgacgctggcggcgaagtggccggcgcccctcagcggctcggagtccaggaggggcggggcgagggcgtcgttcttgcgctggcggccgttggagctccagaggacgaagccgcaggcggcgacgaccagggtcaggatgaggaccttgcgcttgtagatgcggaagcgcattttgacggtatttgtgttctgaagaacgaaactggcgacttgcgcttaatgaaaactgcaaaaaggaaagttagagacgatgtacttgtcaagaaccagtgtatcgatgatggtatacgcacttgaaagtggcaggtgattgcttgaatcaggtatgccgacgaaggaagtgaagaggaagaatcgaagaagatttgaggaggggtcgaagtttttatgggcaggcgggaggggcaaggcgggttggtggggttacgcagcacaaaaacttcaaccacggtggggcaagccctcgaatttcgccaggaatttcacgtcgtgctttgccgcccagccccgcgattcaggcttcacttcctccacaaccgcatgcaaatcgccagagggctgcatcccggtattttgattcgcacccgacaagtaatgatagggctctaaatacagctagaggagtccgcaacgcgatgatggactctttgaacaattattaagtacagtcttctctttctcttcagaccagcgcaacacacccctccagatttgaaactgcccctctcgcgctcgcacagggaactgaactggcaagcccctctttttgccaatcgtttgagcctccctccctgcccttactgagcattggccgcctctggtgccactacttggtatggaactggcatgtcacgctggcctttaagtgggacgttgcgctgtggcccccggtccgttaggcattggcgctcagcccaaccttacgaagttgttcatggaataatggaggacggatttgcagcctgcggcgtgagtcaactaccctccgatcatgcaaatccgcctcccattttctgcaagtttgaagtccacattctgcagcaacgagatgttgtttttatccatacaacgctcctcctaaaatgacaaaagtattattgttcggcggtttgtttgtttgcctgggggcggtcgcagggccgtcgatgttgtggcaaaagtggcccactcatctctctttttttttgtctagatcgtcctggggcaactggtcggttctcttggccagcgatcgctagggcgactgtgtatcggaacagaataggggccatcatgatccttcctcctccgagtcactgggtggcagcttgccgacagttgagtcctcgtcctcgtcctcgccgaacgtgttcttgacaaactccttgggcctgtgcagcgatgttagaggcgactcggaagcatacgacaagcatggaggacatgccagtaaggctgctttcgccactccttctcttcgcgcacgatgttgatgatttcgcccaccaccttgctagccttcgaccgctcggccgcggacgccagatctaccagcacatacccgccgcgcctgatgaagatggtgttccgaaactttggctcgagctcgacgaggacaggctttccgttgggaagctcgcatgtgtaaaggctgttgccttcggccttttggacgcgcgcaacgaattgggtctcggtaagctcgtcgggaggggtcgctgactcctgcgctgcagcgaggacagcacgcttcggttttcccatgtttgtcgcgagtttcggttcgctttgggtgagcggagagcttccgtcatcagacggcacgcaatgacccccgccatggaattttagactttggtggggccccttttctgcccctcaacgcgaaaagcccgcgcaacgccccacgttggtgccattaccggccctcctgccatgtgataaccccctacattccttccatcactcccctttcccttgcagcatcatgcaggatccaatgaatgataggtccttcatcggtggctcctcggaaggccgctcgaacccccgttgtcagcgccggtgcgagattatccgactctccggcagctcggggccgacaccggcacctcactcccggacccccctctctgcaagggcccattttaaaacggcacaatggctgccgaagggtgcggccagggaggatgatcgacagcgcgggccaagaccagttagcggggccgaatgtcgccttcgtcctcgtctccttctgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 25 - DNA, 19105 bp, synthetic construct
  source 1..19105: mol_type = other DNA
ggcgcgccgtttaaacgaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagggcgacggcctcggccagcagtacaactcgcccagcaagttgatcaacatttgcggcaccgacattgtcatcgtagggcgtggcatcaccgccgccggcgacccgccctccgaggctgagaggtacaggagaaaagcctggaaggcctatctggcgcgtctggcgtgatttggggggagggggagaggagatgggggacgggaggggtcgccttggtcagtcttgtgcgtgtcctgcagcggattcgtcaccggggcagcacccaaaagagggagaaaaaggggaaaaaaaataaataaataaaaagggttaagttgttgaaaaaagtgttgtgagctctctggcaaggcgcgccatttaaatcgttggcggtaacagcaggtgccgttcgctgctgctcggtccttttcccgaaagtaccctgccttcctggcctcgacctccgcctgcggggctctgggcactgtcaaactttcgcagttttgcacccgcagcgattcgagcgggacccgcttcgccaacgcggatcaggagaggtaaaagtctcccctgtcctttctgccggagcatcctccgtccgtcgttcgctggttgttgtgtgtgtgtgtgtgtacagtactgtaggtacatagtgctcaccccagggcatttccccaatccgcgattgcgcataccataggcgtcatctgaatctgcgttacgtccggataacactacgcagtacgaaggagtcggtgctactacgcccgcaagttcaagtctacggtgtggctaaggtgctcaagggcatagttacacgcaccccgggcgagcttctcgcacctcgaccctttgccggggcgtcactggtccgacaactcatttaaatcatttccacaactcatgccgagagaagtgtcagactgggcaactaaagtagtagtagtaatagctcgattaccatgatgaaatgctgggcgtcgaagcagctgcaggtccggcatgcagcaatccccactccgctccatcggctgctgttctggggtcaatccgtaccctcccaagttcacctcgccgctgacctcgggatcagctgcgttgtgcattcatgaataatgcgcaacatgagcaacccaacttcatcaagggagtttcgacgtcaccatatcaccacacatcttggaacagaattggggacaaggcagctggattgacggggaaatacataaaccggacgacctatgacgaccatcctatcccgtcaccgactccgatcccctgcggatgatggctcaaagaaccaagtttgatgagccggctgtgtgtcccagcacatacaacaaccgaagtatcagccccgttccgaacgcaggatcccagtctaccgaatcgattttggacagcccgagagaagccaaacaccgaaccgaaggaggaattgtcggaagacgtgcattatgagtcgttcacgcgcgcgttagcggcggttctccgactcgcgcatccggcgcatgtactcctcctggtagctgccgacgttgttcttgcggtcggcgttggtgacctggtcgaaggggacgcggtgggccagcatctcctggatctccttggcgggcgggtactggcccgggcagatccaggagccgggcgggtggcagacgcggttggccgggtcggcgacccacttcttgctctcggcggagacgttcatgaccttgaagatgcggaccaggccgtacttggaggagtagacttcctggaagagcgaggggttgaccttgacgcccttgcgcttgccggcctcgtgcaggttgtacaggagggaggcgcgcatcatcggggtggggcggctgtagtcgttgcggtggaagccgaactgctggcacagcgggtcgtccgggcagatgtcgtggtagacggagttgccgatgcgggccatgtgcgggctcttcatcaggtcgcccgactggccggcccagatgaggacgtagtcggccatgtggcggaccaggctgtgggcctcgacgaccgggctggtgagcatcttgccgatggtggcgatgtgctcgtggttccaggtgttgccgtcggccagcgaggtgcggttgccgatgccggtgatctggtagccgtagtcccaccaggcgaggacgcgggcgtcctccggggtgctgtcgcgcagccactcgtaggccttcaggtagtcgtcgacgagcaggttcatcggcttgccggtggcgcggttctggacgacggcggcgaagacgatcatggggttgctggactgctcggcgaacttggtcgagtggctggcgaactccgagctgaagaagctgacggcggtggtggtgaccagggcccacatggcgatggacaggaccatgcggtggccccaggccagggaggagccggcgaagacgtcgcagaaggcgcgggcggtggtggcgttcttggcgtcgtcgcggcccgagcccttgccggcgcccctctggtggcgctgggcctgcttctgctgcttcttggccttggtggcgtcgctgtcccagaaggagagctggacggcggcctccaggatggtgccgacgaagatgccggtcgacaggcaggcggcggggcccgacaggagcaggaggcgggccatgcgggtggagaagtagtagacggcgcccgagttcaggagccagaagaccttggagggcgagtagtggacgaaggtcgagacggccaggacgatgctgcccaggccccaggtgacgccgcagacgtggaggaaggcccacatggcctcgggcgaggccggctggtgctcggcgacggagtcgacgagcgggttgccggtgcgggtgtgctcgacgaagagggcgcggacgcggacgctcagggggccgaagtagccggtgggggcgaggaccgagatggccagggcggcgacgccggccatgacgctgaagacgcggacgcggatcttgaagttggcgcgggagcggacctcgacgccggcgcgggcgcggaggacctcgcagacctggaggccgcacaggaagacgaggaccaggagggcgcccagctgctccagggacttgaacgggctcatgccgacgggcgggacgcagacggcgatggcggtgccgacgacgtagaagagggtgtaggcgcgcaggaggctggggttgtaggtgttgcgggcccagtcgaccatgctggagatgccggcgtgcatggcgaccatgttcaggacgaagatgtagccgccccaggcggcggccatgtagccgtaggcgacgccggtcaggacgccgatcggccaggagctgcgggtgcggagggagcggacccagcagtagaaggtcaggagcatggcggcgacggcgatgcactcgttgtcgaactcgccggccatggagcgcatgaggtgggccgggatgatgctgaaggacagggcggcggcggcggcggcgacggtggagcccgaggcctcgtaggtgcagaaggcgagggtggcggtggcgatggcgccgaaccaggcgggcatcaggacgcagacgttgttcagcgacatgggcatgccggcggcggccagggcgcggtggatggcgacggcggtcagctggaggcccgggtaggtggtgctgccgacgggcctgccgagcgggtaccaggacatgtagtcgaaccaggagaagaaggccgaccagccgtgggtgctcatgtactcggcggcgcggtagttgaaccaggggtcgaactcgtggatgaggtagccgtagatctggacggagatcatgcggacggtgaaggcctggaagcaggaggcggccaggacgaagagggcgacgacggtcaggacgaagtggaccggccagaacgggaaggggaagatgccgatgaagtccttctcgtcggtcagggtcttgggcaggaggatgaccttggcgggcgggctggcggtcttggtctgggaggcggcgtcctcggcctgggcggaggcctcgcggctggcggtggcggcggagccggagtcgcccagggagttgcccttgcgcttgcccatgttgcttgcgtggtgtgctggaagctgagtgtattaggtggattgacaagtccctgcgggcaacgggaccgagtgagcaagccaggatcaggcgagcaagaggcaggtggtctgattctatcaacctacgtttagagacttgagatggaccagggaatgggcgttttgttttcgaattgatggtttacgatggatttcgttggacggaagaccgatgaggggaaaggagaggagaagcccaaagagggggtcggaggtggcctttattaagaggcggccggccgggcaatgggcagatcagccatctttgctgcatcgttcctgcgactgtttcgtcagaccgggcggggtaatgtcaggagagctccctggtaggttgcgggcagcggcagtgatcacgttgactggctcacgggatcgcgtgacgagtacatcatgatggcacgacctcgcaggcgagccctgcgtggcttaaacaggccaaggtacctggcccgagcgtcctgcagccagcgctaacagcccagcccaagccagaatctggggtaatctggggtaccggggtgcccgacccactgcgggcaaccagcgcttgtgcaccgcgtaaggcctcaacaagacatcagttagtatcgatgccgagattcagttggcaattacatacgtctaacttttccaatgcttattttgagtttcttgtagttatgcagctggtggaagttaggacaggaaccttgagtgacaagcaatccggccgggccgggaaggtgcccgctgcacgatcagtggggcaaaggtggggtatcccgagcaggagcgaactccaacagagtattcgatcaaaaaaggcaagtcctcccccaccatcctttgtagcttgcaatgcatctccttttttgcaatggattttgcttcgcgagtgctaatgccttgtgaaggactatgtggttggttcaaacctgttgttttgatccatctagtccacgttgcaggcatacaaatacccgacttaattaagagtgtaggatgatggtggcaaaactttgaccgacgagacgagacgtgttcacggaggcacgggaggtaatgaatgctctgcttctcgaagcttgcgctctcttgcagcaagctgcatcatgaggatcattgacgtgctggcttgtcggcaacaacctgcattgtcggaggaaggatgtgaaaggcggttacgaagccggaacatccaagtttgcattgccaaggctaggacggagcaggaactgtttttcacatgcaacttccgaaagcgggggagacccccgccatcccataggcatcaaatgtcatcgcaccacgattacgcagcatcaaacgaacaataatcgggggactccaaacgagtctattcatccgtggtatctccatgttaccgtgtcattaatggtaaaccccaaatttccagcggcagccaatggttggccgtcccaaacccgctaaaacgctcccaggcccattcccgtccgatgcagacactttagagctcgtcgtgagcaaggtggccgccccgtcgtgatgatgaacggatgctaaaggggtgcgcctccgtgttaaagacaaatttgttcccgccgttggcctgcacctgcacatccgactcctccgcaaagatcaggtacgcatacttgagcgcctcggcaaaccagaagctctccatatcgtcagaggcacccccgccgttggcctgcgtcacgtcgttgatggacgagtacgcgctgccggcgcggcatgcgtcctcaatggcactgaacgcttcccacgccaggtcctgccacttggagtcgcccgtgacgcggtatgcgtagtacaagctctccagcgtctccggccgcaggatgtaatacggtgccgtcacccagaatcctgccgacgagtagaacccggactgggacgagggcggcgagccgccggcgcccgtcacgctgtccacccacgcgaagccttcggggccgattccagaagccgtctggttgtacgtggcaaagtacgagctggcaagcttgattccaaagtcaatgtacttttgctcgttcaggagaatgcctcccaagatgaagttgccaccggcaaaactggccactacaaggcaagaaaagggggagactgttagcacacaatcaccaccacgtacgacgtctcgagatgactgtcgaatggagaaacttacaatgtcctgagtttggcgacgtagactgtccgttgtacgaagacaaaaaggtcaagtccttgcgcgtcgacgggtgagaggcgagatgcgcaatggtcgagtcggcagcaaggacccagcgatccttgtagtgtgcaaacgcaaccgggtcgtacaggtacatcttgatcaggtactcgtagaagctgtccatgaggccggaccagctgccgctgctatcctgaaaggtaccgttgctcgtgctgacaaacgttccaatcaggccaggccatgcctccgggcttccctttggattcaggagatacgactcgcccttctgcgcaagctgggcatactgcgggtttcccgtcaggtcgctcaaccgtgtccactcgagcaccaggcttccaatttcagcgacgttgttgctagatgcaccacttctccggacagtagggttgaagaagacggtagggtccgggacaccgctgggagtggtgaacgcaaccttgaggccgttggccagtgtttgagcctgcctcagaaggctgtttaccagggtctggtttgtcgccaaggagctgaaaggacctcgcaacaggtcataggctaggtagaatcatcgattagccataccgtcagcgccgaacaatccttgacgcggaaactaggctcgcccatcgaacacgtaccagaaagcaggccaccgaggtaccgaatgttggtctcgaacacggagatgccttggttggcaaccgcagtcgtggtgaagttgatctgcggtacatactgaaggatcgtgttcacaatgtcggcatcccccatgaggatagccgtgtccaagccatcgattgccgacgagccccagccgtttctaggccaaagtaagatacaaattcgccactttatgaaaggactgaggttccgtcacgagctcgcagcttacctctcatcatcaaagctgttgctgaccgggtggaggtcgtcatggggaaaggcaaaatggtggtaagcgttccacgacgtctggaatgcggccttgactgctgccgccctcgtagggttgggagatccacgttttgtggcgcccggctttggatacgccagcgcaggtccgatgagcccgagggcaaggacggagctgctagggaatctcatggtggacgatggacgattgtctgtgtgagtaagggaaaggtggataggaggaaggggaaggaagttctgaggagggatggaagacggatgatgatgtgctagtaggccccaatgagccgaggagggaaggcctgatctcctttatatccaggaaccatcacggccgtttcccgccatgatctgcccccatctctcccgcgagtttcggaccgtcaacaccgatgaaaaaaagctgggcatcgcttgagagatccgggccctgtgttacgctgagtgcacagcgtgggcgatgtgggcagccggggcctcgacaaggatccaagaacaaccccccaccgcggggtaggttgaccctggcgaaggactggcaaacgaagggccgtgaatggccaacggtcagcaatccggggggctgttgatagtgggcaagggccttgcgcgttgcgatgcacgatattgcggagactgaggagccccactagcgcacccttgggccccgccaacccccaacgggccaaagggccagctctgggagcacggaaggagaacccgaaacacatgtttctggaaagagaaagcttgagcatggtatgatagtgtatttttttcctgcctttcttttctttcccaaccccaagttaggcaggtattcccgtgccttacatgggcacctgggagagactattttggcgcggggggggggtaaacctccgaattcgacctctccttccattgttgattgaattgaagaaggtcagctttcgcagttaccgcgccctcgaaaactcgccagaataatcgcgaaagtcgctccgagtcgctctctccaccttcgcatagtttccggatccacggcccgtgaaggacctgacgagcgctccggctgggtaaacctcggctgattctgggggtgaacaaaccaaaaacacgctggtcacgcgcaggggcgtcgttctagctcagcaaacgaaccacggcaccacgccgcccgcgccgagaggagcatctcgcaagccaggctttctagccaaggcacggggtatccatctctctgaggcgagacgagacgcatccgggccaaggagcactttagcgatgcgcgccctctctggcaaaactccggattttgctttcttgccgtggctaaacctcggtcgctctggcgattcacgtcccttcgctcccaggaaaccgaaccgtgcggccgactctgaacgagccgggcggctctcttcttcttttctcttctttcttctcgtcccatacgcttacttggaacactatgggcgacggcaactcgggctggctgttccggagcatggacatcgccgctctgcttatcgagtgtttggtgtgtcagacccgctccatcattccgtgaccgtggcaaccactagtcgcacacctacttgtatacactcccaagcgtagccccaagagcaggaaaagaaaagtgagaaacaagaagagatttgctgctcagagcaacccccagctgcaccctccccaccactcgcgtctccaatttagtggttgccgtgggctcataggttggggtccctatggatttgggggtggacgctgcaggatcggagtaatcatcgaggaggacgacgctgcttgacaggccaggcgtaaaaagaggcaatcatctgatggtgtggtgtgagagaacgggtcaagtaacagtcgggggaacgttggtctttggtttgggttgaatggtacagttgattaaaacaaaaagacagtgacaaggggggaatattctaccaaacaacgtccgcacgacccgcgaggctagccaagaacgcacgacccaaaccacgtccgcagacagggaaaacgagacaaatcacggacaacaggagagaccagccgatttttgttgttgttgttgttttactgcagcctgcggtaggacttgcagagctcgtggtcgcggatgtcgccccagccaccgttcttgcgcgggggcgagatggcggccatggggaacttctcgccgatgaccagggtctcggggaagaggtactgcttgttgttgttcaggaggctctcgatctgggcggactgggtgctggggcggcaggtcttcttgtggtgcatgccgcagtcgccggcgtggaagatcctgggggcctgcgggaccaggaccttccagaccttggggaggcaggccagggtgaggtactggagggtccagtcccagttgtagtcgtcgtaggtgcagaaggtgtcggtgcactcgatcagcttctggtaggcgtcgcgggtcagggccaggcccatgttgtgctcggtcgacttccaggtcttgacgtcgaccttgtcggcgatgccgtagaaggagcggatggtggtgtaggtgcccagggagaggacgtcgcagccggggcactcctgctgcttgagcttccacatcttcttgaagacgtggtagaagtcgggggccaggtagtggtcttcctccaggaagaggatcaggccggtgtagtcctggaggaccttgacgcgctcccagacgaagtgcagcttccaccaccagtggtgcttggtctgcgagaacttggcctcgcggtagtggccgaagctgtcggggtactcggcgttgatgcagccgagcttcagggcggcgttcttcttgaggtcgcgggggcagtcgcgggggtccgagccggggaactcggaggggtagagctggatgctgaacgggaagaagacctgcaggacggggcagaagtcgacgctggagatgagcgagttgatctcggcgctccagaagtcgtgggagaagatgacgaggacctcgcggatgccctgggccttgcggagcgagtcgatcaggaggcggaggtactccgggcggttgtggacctggacgacgaggaccagctcgcccggggaccaggtgccgtccttgtcgacgttgcggagcatctggtcgaagttcagctggtagacgaggctgcggtagcggagggtcaggttgtcgacctccgggcgggggacggcggggacgagcggggcggccgagtcgttcgagaccctgcggatgccgacgctggcggcgaagtggccggcgcccctcagcggctcggagtccaggaggggcggggcgagggcgtcgttcttgcgctggcggccgttggagctccagaggacgaagccgcaggcggcgacgaccagggtcaggatgaggaccttgcgcttgtagatgcggaagcgcattttgacggtatttgtgttctgaagaacgaaactggcgacttgcgcttaatgaaaactgcaaaaaggaaagttagagacgatgtacttgtcaagaaccagtgtatcgatgatggtatacgcacttgaaagtggcaggtgattgcttgaatcaggtatgccgacgaaggaagtgaagaggaagaatcgaagaagatttgaggaggggtcgaagtttttatgggcaggcgggaggggcaaggcgggttggtggggttacgcagcacaaaaacttcaaccacggtggggcaagccctcgaatttcgccaggaatttcacgtcgtgctttgccgcccagccccgcgattcaggcttcacttcctccacaaccgcatgcaaatcgccagagggctgcatcccggtattttgattcgcacccgacaagtaatgatagggctctaaatacagctagaggagtccgcaacgcgatgatggactctttgaacaattattaagtacagtcttctctttctcttcagaccagcgcaacacacccctccagatttgaaactgcccctctcgcgctcgcacagggaactgaactggcaagcccctctttttgccaatcgtttgagcctccctccctgcccttactgagcattggccgcctctggtgccactacttggtatggaactggcatgtcacgctggcctttaagtgggacgttgcgctgtggcccccggtccgttaggcattggcgctcagcccaaccttacgaagttgttcatggaataatggaggacggatttgcagcctgcggcgtgagtcaactaccctccgatcatgcaaatccgcctcccattttctgcaagtttgaagtccacattctgcagcaacgagatgttgtttttatccatacaacgctcctcctaaaatgacaaaagtattattgttcggcggtttgtttgtttgcctgggggcggtcgcagggccgtcgatgttgtggcaaaagtggcccactcatctctctttttttttgtctagatcgtcctggggcaactggtcggttctcttggccagcgatcgctagggcgactgtgtatcggaacagaataggggccatcatgatccttcctcctccgagtcactgggtggcagcttgccgacagttgagtcctcgtcctcgtcctcgccgaacgtgttcttgacaaactccttgggcctgtgcagcgatgttagaggcgactcggaagcatacgacaagcatggaggacatgccagtaaggctgctttcgccactccttctcttcgcgcacgatgttgatgatttcgcccaccaccttgctagccttcgaccgctcggccgcggacgccagatctaccagcacatacccgccgcgcctgatgaagatggtgttccgaaactttggctcgagctcgacgaggacaggctttccgttgggaagctcgcatgtgtaaaggctgttgccttcggccttttggacgcgcgcaacgaattgggtctcggtaagctcgtcgggaggggtcgctgactcctgcgctgcagcgaggacagcacgcttcggttttcccatgtttgtcgcgagtttcggttcgctttgggtgagcggagagcttccgtcatcagacggcacgcaatgacccccgccatggaattttagactttggtggggccccttttctgcccctcaacgcgaaaagcccgcgcaacgccccacgttggtgccattaccggccctcctgccatgtgataaccccctacattccttccatcactcccctttcccttgcagcatcatgcaggatccaatgaatgataggtccttcatcggtggctcctcggaaggccgctcgaacccccgttgtcagcgccggtgcgagattatccgactctccggcagctcggggccgacaccggcacctcactcccggacccccctctctgcaagggcccattttaaaacggcacaatggctgccgaagggtgcggccagggaggatgatcgacagcgcgggccaagaccagttagcggggccgaatgtcgccttcgtcctcgtctccttctgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg